(19) United States
(12) Patent Application Publication
(10) Pub. No.: US 2023/0203441 A1
(43) Pub. Date: Jun. 29, 2023

(54) CELLS FOR TREATING CANCER

(71) Applicant: LIfT BioSciences Ltd, Brighton (GB)

(72) Inventor: Alex Blyth, Brighton (GB)

(21) Appl. No.: 17/785,012

(22) PCT Filed: Dec. 11, 2020
(86) PCT No.: PCT/GB2020/053199

§ 371 Date: Jun. 13, 2022

(30) Foreign Application Priority Data
Dec. 12, 2019 (GB) 1918313.6

(51) Int. Cl. C12N 5/0787; A61K 35/28; A61K 35/15; A61P 35/00; G01N 33/50; G01N 33/569

(52) U.S. Cl. CPC C12N 5/0642; A61K 35/15; A61K 35/28; A61P 35/00; G01N 33/5011; G01N 33/5047; G01N 33/56972; C12N 2506/11; G01N 2800/52

(57) ABSTRACT

The invention relates to a method for determining the suitability of a granulocyte for treating cancer. The invention also relates to said granulocytes, methods for identifying said granulocytes and stem cells capable of differentiating into said granulocytes, compositions and kits comprising the same, as well as uses of the same for treating cancer.

The present invention relates to a cell-based therapy suitable for treating cancer.

Cancer is a leading cause of morbidity and mortality worldwide, with an annual increase in cancer incidence in developed countries. The World Health Organisation stated that in 2012 alone there were approximately 14 million new cancer cases (and 8.2 million associated deaths), with a projected rise to 22 million cases over the next two decades. Current therapeutic strategies include combinations of surgery, radiation, and cytotoxic chemotherapy, however many of these treatments are ultimately ineffective and associated with harmful side-effects.

Safety and efficacy has been assessed for Haematopoietic Stem Cell Transplantation (HSCT) as a therapeutic technique for treating certain cancers, such as Renal Cell Carcinoma. However, this treatment is still largely seen as experimental due to potentially fatal safety issues, with recipients exhibiting severe Graft vs. Host Disease (GVHD) as a result of the uncontrolled multiplication of pluripotent stem cells. Thus, there is a need for improved and alternative cancer therapies.

In spite of the increased cancer incidence, it has been observed that approximately 50-60% of individuals do not develop cancer in their lifetime. Indeed, in rare cases, some individuals exhibit spontaneous cancer regression. This observation has led to the study of white blood cells from spontaneous regressor individuals, and use of said white blood cells in Leukocyte Infusion Therapy (LIFT).

Conventional LIFT is carried out using apheresis for direct transfer of granulocytes (e.g. neutrophils) taken from the donor to the cancer patient. Conventional approaches currently used in the clinic are not practical or scalable for use as a credible cancer therapeutic. First, granulocytes such as neutrophils have a very limited shelf-life (typically less than 24 hours) making them difficult to store. Secondly, apheresis requires approximately 5 (very rare) donors in order to acquire the required cell number. Thirdly, to avoid an allogeneic immune response from repeat exposure, the same donors cannot be used in a subsequent administration, thus requiring an increased pool of appropriate donors. Fourthly, it cannot be realistically expected that donors will be available on request, or willing to provide an endless source of granulocytes for the LIFT procedure. Thus, there is need to quickly and reliably identify suitable donors that produce granulocytes with sufficient cancer killing activity.

Identifying donors that produce such granulocytes has conventionally been performed using functional assays that measure a percentage of cancer cells killed. However, such assays can be time-consuming. Thus, there is a need for improved/alternative assays that can sensitively and specifically and/or rapidly identify such granulocytes and donors and/or validate any results obtained using functional assays.

The present invention provides a solution to at least one of the problems described above.

The present inventors have surprisingly identified a number of genes (and expression levels thereof) that are associated with a granulocyte’s suitability for treating cancer. Advantageously, the expression levels of such genes can be determined using transcriptomic, proteomic or genomic techniques to sensitively and specifically identify and/or rapidly identify granulocytes with therapeutic efficacy and/or donors producing such granulocytes.

Moreover, by determining the expression of the one or more genes described herein, the present invention allows for the preparation of substantially homogenous populations of granulocytes suitable for treating cancer (e.g. where at least 90% of the granulocytes present are granulocytes suitable for treating cancer).

In one aspect the present invention provides a method for determining the suitability of a granulocyte for treating cancer, the method comprising:
a. comparing a measured expression level of one or more genes by the granulocyte, wherein the one or more genes are associated with suitability for treating cancer and are selected from: CTSG, CAP37, ITGB1, CYBB, SYK, DOCK8, COMP, ATG7, SLC2A1, GZMK, ATM, IKBKB, BCAP31, TAPBP, PPP3CB, ANXA1, PERM, PLEC, ACSL1, RAC1, GM2A, and PSMB2, with the expression level of the same one or more genes in a reference standard; and
b. determining the suitability of the granulocyte for treating cancer based on the comparison.

Representative sequences for the genes for use in the invention are described in the Sequence Listing herein, together with the appropriate Ensembl Accession numbers. A gene for use in the invention may be one or more shown as SEQ ID NOs: 1-24 or 83-87 or a variant thereof. A gene for use in a method of the invention may comprise (or consist of) a nucleotide sequence having at least 70%, 80%, 90% or 95% sequence identity to any one of SEQ ID NOs: 1-24 or 83-87. Preferably, a gene for use in a method of the invention comprises (more preferably consists of) any one of SEQ ID NOs: 1-24 or 83-87.

In one aspect the present invention provides a method for determining the suitability of a granulocyte for treating cancer, the method comprising:
a. measuring an expression level of one or more genes by the granulocyte, wherein the one or more genes are associated with suitability for treating cancer and are selected from: ITGB1, CYBB, SYK, DOCK8, COMP, ATG7, SLC2A1, GZMK, CTSG, ATM, IKBKB, BCAP31, TAPBP, PPP3CB, ANXA1, PERM, PLEC, ACSL1, RAC1, GM2A, CAP37, and PSMB2;
b. comparing the measured expression level with the expression level of the same one or more genes in a reference standard; and
c. determining the suitability of the granulocyte for treating cancer based on the comparison.

In another aspect the invention provides a method for identifying whether or not a donor produces granulocytes suitable for treating cancer, the method comprising:
a. comparing a measured expression level of one or more genes by a granulocyte comprised in a sample obtainable from the donor, wherein the one or more genes are associated with suitability for treating cancer and are selected from: CTSG, CAP37, ITGB1, CYBB, SYK, DOCK8, COMP, ATG7, SLC2A1, GZMK, ATM, IKBKB, BCAP31, TAPBP, PPP3CB, ANXA1, PERM, PLEC, ACSL1, RAC1, GM2A, and PSMB2, with the expression level of the same one or more genes in a reference standard; and
b. identifying whether or not the donor produces granulocytes suitable for treating cancer based on the comparison.

In a related aspect the invention provides a method for identifying whether or not a donor produces granulocytes suitable for treating cancer, the method comprising:
a. measuring an expression level of one or more genes by a granulocyte comprised in a sample obtainable from the donor, wherein the one or more genes are associated with suitability for treating cancer and are selected from: ITGB1, CYBB, SYK, DOCK8, COMP, ATG7, SLC2A1, GZMK, CTSG, ATM, IKBKB, BCAP31, TAPBP, PPP3CB, ANXA1, PERM, PLEC, ACSL1, RAC1, GM2A, CAP37, and PSMB2;
b. comparing the measured expression level with the expression level of the same one or more genes in a reference standard; and
c. identifying whether or not the donor produces granulocytes for treating cancer based on the comparison.

In a preferred embodiment, the methods referred to herein comprise measuring, and/or comparing a measured expression level of GM2A, PLEC, CYBB, DOCK8, and/or PPP3CB and optionally one or more further genes. Most preferably, the methods referred to herein comprise measuring and/or comparing a measured expression level of GM2A and optionally one or more further genes. Advantageously, the expression of said genes is highly statistically-significantly different between granulocytes that are suitable for treating cancer and granulocytes that are unsuitable for treating cancer. Thus, measuring, and/or comparing a measured expression level of at least one of those genes has particularly high predictive value. Thus, a gene used in any method described herein may be GM2A, PLEC, CYBB, DOCK8, and/or PPP3CB and optionally one or more further genes. Thus, a protein used in any method described herein may be GM2A, PLEC, CYBB, DOCK8, and/or PPP3CB and optionally one or more further proteins. Most preferably, GM2A or GM2A and optionally one or more further genes/proteins.

In one embodiment, the methods referred to herein are in vitro methods, such as ex vivo methods.

The term “donor” as used herein refers to a subject (suitably a human subject) from whom a sample is obtainable (e.g. obtained). Any suitable sample from which a stem cell or granulocyte cell is obtainable may be obtainable from the donor. The donor may be selected based on one or more of the following characteristics: sex, age, medical history, and/or blood group type. In one embodiment, a donor may be selected if said donor is a healthy donor. In one embodiment, a donor may be selected if said donor does not have cancer. In one embodiment a donor may be selected if said donor is a male. In another embodiment a donor may be selected if said donor is aged 18-55 and preferably 18-35 (more preferably 18-24). Suitably, a donor may be selected if said donor is a male aged between 18-55 and preferably 18-35 (more preferably 18-24). Without wishing to be bound by theory, it is believed that males in early adulthood have a higher likelihood of producing granulocytes (e.g. neutrophils) that are suitable for treating cancer.

The term “measuring” as used in reference to expression of one or more genes of the invention encompasses measuring both negative (e.g. no expression) and positive expression (e.g. expression). In one embodiment the expression is positive expression.

Measuring expression may be carried out by any means known to the person skilled in the art. In some embodiments expression may be measured using high-throughput techniques. For example, measuring expression may be at the level of transcription (e.g. transcriptomic techniques) or translation (e.g. proteomic techniques). Alternatively or additionally, the invention may employ the use of genomics, e.g. to detect the presence or absence of single nucleotide polymorphisms (SNPs), promoter sequences, gene copy number (e.g. duplications), and/or enhancer or other relevant genetic features, preferably those that determine the expression level of one or more genes of the invention. High-throughput techniques can be used to analyse whole genomes, proteomes and transcriptomes rapidly, providing data, including the expression levels, of all of the genes, polypeptides and transcripts in a cell. Proteomics is a technique for analysing the proteome of a cell (e.g. at a particular point in time). The proteome is different in different cell types. Typically, proteomics is carried out by mass-spectrometry, including tandem mass-spectrometry, and gel based techniques, including differential in-gel electrophoresis. Proteomics can be used to detect polypeptides expressed in a particular cell type and generate a proteomic profile to allow for the identification of specific cell types.

In one embodiment, mRNA of a target gene can be detected and quantified by e.g. Northern blotting or by quantitative reverse transcription PCR (RT-PCR). Single cell gene expression analysis may also be performed using commercially available systems (e.g. Fluidigm Dynamic Array). Alternatively, or in addition, gene expression levels can be determined by analysing polypeptide levels e.g. by using Western blotting techniques such as ELISA-based assays.

Thus, in one embodiment, gene expression levels are determined by measuring the mRNA/ cDNA levels of the genes of the present invention, such as RNA sequencing (RNA-Seq).

In a preferred embodiment, gene expression levels are determined by measuring the polypeptide levels produced by the genes of the present invention, such as by way of mass spectrometry, e.g. liquid chromatography and mass spectrometry (LC-MS/MS).

In one embodiment a granulocyte (or stem cell) for treating cancer may be detected using an enzyme-linked immunosorbent assay (ELISA) or a Luminex assay (commercially available from R&D Systems, USA).

Thus, in one embodiment a method of the invention comprises measuring and/or comparing an expression level of one or more polypeptides by a granulocyte, wherein the one or more polypeptides are selected from: CTSG, CAP37, ITGB1, CYBB, SYK, DOCK8, COMP, ATG7, SLC2A1, GZMK, CTSG, ATM, IKBKB, BCAP31, TAPBP, PPP3CB, ANXA1, PERM, PLEC, ACSL1, RAC1, GM2A, CAP37, and PSMB2.

Representative sequences for the polypeptides for use in the invention are described in the Sequence Listing herein, together with the appropriate UniProt Accession numbers. A polypeptide for use in the invention may be one or more shown as SEQ ID NOs: 25-82 or a variant thereof, such as a transcript isoform therefore. A polypeptide for use in a method of the invention may comprise (or consist of) a polypeptide sequence having at least 20%, 30%, 40%, 50%, or 60% sequence identity to any one of SEQ ID NOs: 25-82. In one embodiment a polypeptide for use in a method of the invention may comprise (or consist of) a polypeptide sequence having at least 70%, 80%, 90% or 95% sequence identity to any one of SEQ ID NOs: 25-82. Preferably, a polypeptide for use in a method of the invention comprises (more preferably consists of) any one of SEQ ID NOs: 25-82.

In one embodiment a method of the invention comprises measuring and/or comparing an amount of one or more polypeptides produced by a granulocyte, wherein the one or more polypeptides are selected from: CTSG, CAP37, ITGB1, CYBB, SYK, DOCK8, COMP, ATG7, SLC2A1, GZMK, CTSG, ATM, IKBKB, BCAP31, TAPBP, PPP3CB, ANXA1, PERM, PLEC, ACSL1, RAC1, GM2A, CAP37, and PSMB2.

In one embodiment a method of the invention comprises measuring and/or comparing an expression level of one or more polypeptides by a stem cell, wherein the one or more polypeptides are selected from: CTSG, CAP37, ITGB1, CYBB, SYK, DOCK8, COMP, ATG7, SLC2A1, GZMK, CTSG, ATM, IKBKB, BCAP31, TAPBP, PPP3CB, ANXA1, PERM, PLEC, ACSL1, RAC1, GM2A, CAP37, and PSMB2.

In one embodiment a method of the invention comprises measuring and/or comparing an amount of one or more polypeptides produced by a stem cell, wherein the one or more polypeptides are selected from: CTSG, CAP37, ITGB1, CYBB, SYK, DOCK8, COMP, ATG7, SLC2A1, GZMK, CTSG, ATM, IKBKB, BCAP31, TAPBP, PPP3CB, ANXA1, PERM, PLEC, ACSL1, RAC1, GM2A, CAP37, and PSMB2.

In one embodiment a method of the invention employs a genome wide association study, which is compared to a reference standard (e.g. a reference standard from a reference population, such as a reference standard from: a suitable or unsuitable donor, or a suitable or unsuitable granulocyte, or a subject that is suitable or unsuitable for treatment with a granulocyte or stem cell of the invention, or a subject that is at risk or not at risk of cancer or combinations thereof).

Methods suitable for establishing a baseline or reference value for comparing expression levels are conventional techniques known to those skilled in the art.

The term “increased” as used herein in reference to expression of the one or more genes of the invention may refer to an expression level that is statistically-significantly increased when compared to a reference standard. Such a gene may be considered to be upregulated.

In one embodiment increased expression means greater than 1-fold, 1.25-fold to about 10-fold or more expression relative to a reference standard. In some embodiments, increased expression means greater than at least about 1.1-fold, 1.2-fold, 1.25-fold, 1.5-fold, 1.75-fold, 2-fold, 4-fold, 5-fold, 10-fold, 15-fold, 20-fold, 25-fold, 30-fold, 35-fold, 40-fold, 50-fold, 75-fold, 100-fold, 150-fold, 200-fold, or at least about 300-fold expression when compared to a reference standard.

The term “decreased” as used herein in reference to expression of the one or more genes of the invention may refer to an expression level that is statistically-significantly decreased when compared to a reference standard. Such a gene may be considered to be downregulated.

In one embodiment decreased expression means less than -1-fold, -1.25-fold to about -10-fold or more expression relative to a reference standard. In some embodiments, decreased expression means less than at least about -1.1-fold, -1.2-fold, -1.25-fold, -1.5-fold, -1.75-fold, -2-fold, -4-fold, -5-fold, -10-fold, -15-fold, -20-fold, 25-fold, -30-fold, -35-fold, -40-fold, -50-fold, -75-fold, -100-fold, -150-fold, -200-fold, or at least about -300-fold expression when compared to a reference standard.

The fold change difference can be in absolute terms (e.g. CPM: counts per million) or Log2CPM (a standard measure in the field) of the expression level in a sample. Preferably the fold change is Log2 fold change. In one embodiment a Log2 change is an increase of at least 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6 or 2.7. In one embodiment a Log2 change is a decrease of 0.1 or more, 0.2 or more, 0.3 or more, 0.4 or more, 0.5 or more, 0.6 or more, 0.7 or more, 0.8 or more, 0.9 or more, 1.0 or more, 1.1 or more, 1.2 or more or 1.3 or more. A decrease may be indicated by the presence of a “-” symbol prior to the value.

In one embodiment said fold-change is measured and/or is determined by RNA sequencing (RNA-Seq), e.g. in toto.

The term “unchanged” or “the same” as used herein in reference to expression of the one or more genes of the invention may refer to an expression level that is not statistically-significantly different to a reference standard. Preferably, an expression level that is the same as a reference standard.

The expression level may be an average such as a mean expression level. In one embodiment statistical significance is determined using two-way ANOVA, e.g. where n is at least 3 and data are presented as mean +/- standard error of mean.

In one embodiment the methods of the invention comprise measuring expression of combinations of the genes described herein.

The term “one or more” when used in the context of a gene described herein may mean at least 2, 3, 4, 5, 6, 7, 8, 9, 10, 11, 12, 13, 14, 15, 16, 17, 18, 19, 20, 21 or 22 of the genes. Preferably, the term “one of more” means all of the genes. Likewise, the term “one or more” when used in the context of a polypeptide described herein may mean at least 2, 3, 4, 5, 6, 7, 8, 9, 10, 11, 12, 13, 14, 15, 16, 17, 18, 19, 20, 21 or 22 of the polypeptides. Preferably, the term “one of more” means all of the polypeptides.

The expression of one or more of ITGB1, CYBB, SYK, DOCK8, COMP, ATG7, SLC2A1, GZMK, CTSG, ATM, IKBKB, BCAP31, TAPBP, PPP3CB, ANXA1, PERM, PLEC, ACSL1, RAC1, GM2A, CAP37, and PSMB2 correlates with a granulocyte’s suitability for treating cancer. Said genes are therefore referred to herein as genes associated with suitability for treating cancer. Thus, the term “one or more genes associated with suitability for treating cancer” (and the like) may in be synonymous with (and thus replaced with) the term “one or more of ITGB1, CYBB, SYK, DOCK8, COMP, ATG7, SLC2A1, GZMK, CTSG, ATM, IKBKB, BCAP31, TAPBP, PPP3CB, ANXA1, PERM, PLEC, ACSL1, RAC1, GM2A, CAP37, and PSMB2”. Thus, the term “one or more polypeptides associated with suitability for treating cancer” (and the like) may in be synonymous with (and thus replaced with) the term “one or more of ITGB1, CYBB, SYK, DOCK8, COMP, ATG7, SLC2A1, GZMK, CTSG, ATM, IKBKB, BCAP31, TAPBP, PPP3CB, ANXA1, PERM, PLEC, ACSL1, RAC1, GM2A, CAP37, and PSMB2”.

Without wishing to be bound by theory, the inventors believe that, based on the data obtained in the Examples and the inventors’ theorised mechanism of action, the one or more genes may have the following functions making them suitable for treating cancer:
a. killing cancer cells: GM2A, CTSG, CAP37, CYBB, GZMK, ATM, PERM, ACSL1, ATG7, SYK, DOCK8, RAC1, and PSMB2; and/or
b. locating and/or binding to cancer cells: ANXA1, ITGB1, COMP, SLC2A1 and PLEC; and/or
c. recruitment of immune mediators: BCAP31, TAPBP, IKBKB, and PPP3CB.

In a preferred embodiment a method of the invention comprises measuring and/or comparing the expression of ANXA1. Advantageously, the inventors have shown that low levels of ANXA1 expression are associated with high cancer killing activity and therefore suitability for treating cancer. Without wishing to be bound by theory, the inventors believe that ANXA1 modulates chemotaxis and/or motility of granulocytes and, in particular, that low expression of ANXA1 promotes granulocyte motility and thus location/binding to cancer cells.

In one embodiment expression of one or more of ITGB1, CYBB, SYK, DOCK8, COMP, ATG7, SLC2A1, GZMK, CTSG, ATM, IKBKB, BCAP31, TAPBP, PERM, PLEC, ACSL1, RAC1, GM2A, CAP37, and PSMB2 is increased in a granulocyte that is suitable for treating cancer when compared to a granulocyte that is unsuitable for treating cancer. Alternatively or additionally, in one embodiment expression of ANXA1 and/or PPP3CB is decreased in a granulocyte that is suitable for treating cancer when compared to a granulocyte that is unsuitable for treating cancer.

In one embodiment a method of the invention may further comprise measuring and/or comparing expression of one or more genes selected from: S100A9 and S100A8. In one embodiment expression of S100A9 and/or S100A8 may be increased in a granulocyte of the invention when compared to a reference standard, when the reference standard is from a granulocyte that is unsuitable for treating cancer.

In one embodiment a method of the invention comprises measuring and/or comparing expression of CTSG and at least one further gene selected from: CAP37, ITGB1, CYBB, SYK, DOCK8, COMP, ATG7, SLC2A1, GZMK, ATM, IKBKB, BCAP31, TAPBP, PPP3CB, ANXA1, PERM, PLEC, ACSL1, RAC1, GM2A, and PSMB2. Similarly, a granulocyte of the invention may comprise increased expression of CTSG and:
at least one further gene selected from: CAP37, ITGB1, CYBB, SYK, DOCK8, COMP, ATG7, SLC2A1, GZMK, ATM, IKBKB, BCAP31, TAPBP, PERM, PLEC, ACSL1, RAC1, GM2A, and PSMB2 when compared to a reference standard, wherein the reference standard is from a granulocyte unsuitable for treating cancer; or
decreased expression of ANXA1 and/or PPP3CB when compared to a reference standard, wherein the reference standard is from a granulocyte unsuitable for treating cancer.

In a particularly preferred embodiment, a method of the invention comprises measuring and/or comparing expression of GM2A and at least one further gene selected from: CAP37, ITGB1, CYBB, SYK, DOCK8, COMP, ATG7, SLC2A1, GZMK, ATM, IKBKB, BCAP31, TAPBP, PPP3CB, ANXA1, PERM, PLEC, ACSL1, RAC1, CTSG, and PSMB2. Similarly, a granulocyte of the invention may comprise increased expression of GM2A and:
at least one further gene selected from: CAP37, ITGB1, CYBB, SYK, DOCK8, COMP, ATG7, SLC2A1, GZMK, ATM, IKBKB, BCAP31, TAPBP, PERM, PLEC, ACSL1, RAC1, CTSG, and PSMB2 when compared to a reference standard, wherein the reference standard is from a granulocyte unsuitable for treating cancer; or
decreased expression of ANXA1 and/or PPP3CB when compared to a reference standard, wherein the reference standard is from a granulocyte unsuitable for treating cancer.

In one embodiment a method of the invention comprises measuring and/or comparing expression of CAP37 and at least one further gene selected from: CTSG, ITGB1, CYBB, SYK, DOCK8, COMP, ATG7, SLC2A1, GZMK, ATM, IKBKB, BCAP31, TAPBP, PPP3CB, ANXA1, PERM, PLEC, ACSL1, RAC1, GM2A, and PSMB2. Similarly, a granulocyte of the invention may comprise increased expression of CAP37and:
at least one further gene selected from: CTSG, ITGB1, CYBB, SYK, DOCK8, COMP, ATG7, SLC2A1, GZMK, ATM, IKBKB, BCAP31, TAPBP, PERM, PLEC, ACSL1, RAC1, GM2A, and PSMB2 when compared to a reference standard, wherein the reference standard is from a granulocyte (or stem cell) unsuitable for treating cancer; or
decreased expression of ANXA1 and/or PPP3CB when compared to a reference standard, wherein the reference standard is from a granulocyte unsuitable for treating cancer.

In another embodiment a method of the invention comprises measuring and/or comparing expression of ANXA1 and at least one further gene selected from: CTSG, CAP37, ITGB1, CYBB, SYK, DOCK8, COMP, ATG7, SLC2A1, GZMK, ATM, IKBKB, BCAP31, TAPBP, PPP3CB, PERM, PLEC, ACSL1, RAC1, GM2A, and PSMB2. Similarly, a granulocyte of the invention may comprise decreased expression of ANXA1 and:
at least one further gene selected from: CTSG, CAP37, ITGB1, CYBB, SYK, DOCK8, COMP, ATG7, SLC2A1, GZMK, ATM, IKBKB, BCAP31, TAPBP, PERM, PLEC, ACSL1, RAC1, GM2A, and PSMB2 when compared to a reference standard, wherein the reference standard is from a granulocyte unsuitable for treating cancer; or
decreased expression of PPP3CB when compared to a reference standard, wherein the reference standard is from a granulocyte unsuitable for treating cancer.

The term “for treating cancer” as used herein means “suitable for treating cancer”. A granulocyte that is “suitable for treating cancer” as used herein means that a granulocyte is capable of killing at least 51.5% of cancer cells in the “cancer killing activity (CKA) assay” described herein. In one embodiment a granulocyte is capable of killing at least 70% of cancer cells in the “cancer killing activity (CKA) assay” described herein. Preferably a granulocyte is capable of killing at least 80% (e.g. at least 90% or 95%) of cancer cells in the “cancer killing activity (CKA) assay” described herein. Reference to a stem cell “for treating cancer” or that is “suitable for treating cancer” means that said stem cell is capable of differentiating into a granulocyte that is suitable for treating cancer.

In contrast a granulocyte that is “not suitable for treating cancer” or is “unsuitable for treating cancer” is a granulocyte that is not capable of killing at least 51.5% of cancer cells in the “cancer killing activity (CKA) assay” described herein, i.e. a granulocyte that kills less than 51.5% of cancer cells in the “cancer killing activity (CKA) assay” described herein. In one embodiment a granulocyte that is “not suitable for treating cancer” or is “unsuitable for treating cancer” is a granulocyte that is not capable of killing at least 70% of cancer cells in the “cancer killing activity (CKA) assay” described herein, i.e. a granulocyte that kills less than 70% of cancer cells in the “cancer killing activity (CKA) assay” described herein.

Likewise, reference to a stem cell that is “not suitable for treating cancer” or is “unsuitable for treating cancer” is a stem cell that does not differentiate into a granulocyte that is suitable for treating cancer and/or that differentiates into a granulocyte that is unsuitable for treating cancer.

The “cancer killing activity (CKA) assay” or “CKA assay” is carried out using an ACEA Biosciences xCELLigence RTCA DP Analyzer system® according to the manufacturer’s instructions and as follows:
a. 6000 cancer cells are placed in the bottom of a 16 well plate;
b. cells are grown to confluence as determined by plateauing of Cell Index (CI) values (i.e. the ‘normalisation point’);
c. 60,000 granulocytes are added (i.e. giving a ratio of 10 granulocytes to 1 cancer cells) and incubated at 37° C.; and
d. the % of cancer cells killed is the maximum % of cancer cells killed by 48 hours after addition of the granulocytes as determined using the following formula: ((Cell Indexₙₒ_{effector} - Cell Index _{effector})/Cell Index ₙₒ_{effector}) X 100.

The maximum % of cancer cells killed may be referred to herein as “% CKA”.

Preferably the cancer cells are PANC-1 cells, which are commercially available from the American Type Culture Collection United Kingdom (U.K.), Guernsey, Ireland, Jersey and Liechtenstein, LGC Standards, Queens Road, Teddington, Middlesex, TW11 0LY, UK and have catalogue number ATCC CRL-1469.

In a particularly preferred embodminent the term “suitable for treating cancer” as used herein further means that a granulocyte kills less than 15% of non-cancer cells in the “non-cancer killing activity (NCKA) assay” described herein. Preferably a granulocyte kills less than 10% (e.g. less than 5% or less than 1%) of non-cancer cells in the “non-cancer killing activity (NCKA) assay” described herein.

The “non-cancer killing activity (NCKA) assay” or “NCKA assay” is carried out using an ACEA Biosciences xCELLigence RTCA DP Analyzer system® according to the manufacturer’s instructions and as follows:
a. 6000 non-cancer cells are placed in the bottom of a 16 well plate;
b. cells are grown to confluence as determined by plateauing of Cell Index (CI) values (i.e. the ‘normalisation point’);
c. 60,000 granulocytes are added (i.e. giving a ratio of 10 granulocytes to 1 non-cancer cells) and incubated at 37° C.; and
d. the % of non-cancer cells killed is the maximum % of non-cancer cells killed by 48 hours after addition of the granulocytes as determined using the following formula: ((Cell Index ₙₒ_{effector} - Cell Index _{effector})/Cell Index ₙₒ_{effector}) X 100.

Preferably the non-cancer cells are MCF-12F non-cancer cells, which are commercially available from the American Type Culture Collection, 10801 University Boulevard. Manassas, VA 20110 USA and have catalogue number ATCC® CRL-10783™. In another embodiment the non-cancer cells are liver cells (e.g. primary non-transplantable liver tissue cells).

The expression level of one or more genes of the invention may be compared to a reference standard. The comparison may be carried out by any suitable technique known to the person skilled in the art, e.g. a bioinformatics technique. The detected gene expression in the reference standard may have been obtained (e.g. quantified) previously to a method of the invention. The expression level of the genes described herein is suitably known in said reference standard. A reference standard is preferably from the same sample type as that referred to in a method of the invention. For example, both the sample and reference standard may be blood samples.

In one embodiment the term “sample” as used herein (e.g. in reference to a sample from a donor) may be any sample comprising a granulocyte and/or a stem cell and/or other cell capable of differentiating into a granulocyte, preferably comprising a granulocyte. The sample may be any suitable biofluid sample from which a granulocyte and/or a stem cell and/or other cell capable of differentiating into a granulocyte is obtainable, preferably from which a granulocyte is obtainable. A sample may be a blood sample, such as a peripheral blood sample. The term “blood” as used herein encompasses whole blood, blood serum, and blood plasma. Blood may be subjected to centrifugation in order to separate red blood cells, white blood cells, and plasma. Following centrifugation, the mononuclear cell layer may be removed for use in the present invention.

The reference standard may be a proteomic profile (indicating an amount of polypeptide expressed by a granulocyte), a transcriptomic profile (indicating an amount of gene expression by a granulocyte, e.g. measured by way of RNA produced by said granulocyte) or a genomic profile. A genomic profile may be used to detect the presence or absence of SNPs, promoter sequences, gene copy number (e.g. duplications), and/or enhancer or other relevant genetic features, preferably those that determine the expression level of one or more genes of the invention. The skilled person will appreciate that both the proteomic and transcriptomic profiles are measures of gene expression and will employ the appropriate reference standard depending on the technique used to measure gene expression in accordance with the invention. For example, where proteomics is used in practising the present invention the skilled person will employ a reference standard that is a proteomic profile, where transcriptomics is used in practising the present invention the skilled person will employ a reference standard that is a transcriptomic profile, and where genomics is used in practising the present invention the skilled person will employ a reference standard that is a genomic profile. A reference standard may refer to a database (e.g. a genomic database), e.g. which may include data from one or more sources, such as one or more subjects and/or cells.

A reference standard is preferably a reference standard for a granulocyte that is unsuitable for treating cancer (e.g. a transcriptomic or proteomic profile of a granulocyte that is unsuitable for treating cancer). Such a reference standard may be from a subject that does not have cancer (a healthy subject) or from a subject that has cancer. Preferably, such a reference standard is from a subject that does not have cancer. FIG. 1 represents a selection of features that are different between those cells from a subject that has cancer, a normal subject that produces granulocytes unsuitable for treating cancer, and a subject that produces granulocytes that are suitable for treating cancer. Any one of said properties may be measured to characterise granulocytes present in a sample (e.g. in a reference standard).

In one embodiment expression of one or more of ITGB1, CYBB, SYK, DOCK8, COMP, ATG7, SLC2A1, GZMK, CTSG, ATM, IKBKB, BCAP31, TAPBP, PERM, PLEC, ACSL1, RAC1, GM2A, CAP37, and PSMB2 is increased when compared to a reference standard when the reference standard is from a granulocyte unsuitable for treating cancer. In one embodiment expression of ANXA1 and/or PPP3CB is decreased when compared to a reference standard, when the reference standard is from a granulocyte unsuitable for treating cancer. In one embodiment expression of one or more of ITGB1, CYBB, SYK, DOCK8, COMP, ATG7, SLC2A1, GZMK, CTSG, ATM, IKBKB, BCAP31, TAPBP, PERM, PLEC, ACSL1, RAC1, GM2A, CAP37, and PSMB2 is increased when compared to a reference standard when the reference standard is from a granulocyte unsuitable for treating cancer and expression of ANXA1 and/or PPP3CB is decreased when compared to a reference standard, when the reference standard is from a granulocyte unsuitable for treating cancer.

A reference standard may be a reference standard for a granulocyte that is suitable for treating cancer (e.g. a transcriptomic or proteomic profile of a granulocyte that is suitable for treating cancer). In one embodiment expression of one or more of ITGB1, CYBB, SYK, DOCK8, COMP, ATG7, SLC2A1, GZMK, CTSG, ATM, IKBKB, BCAP31, TAPBP, PERM, PLEC, ACSL1, RAC1, GM2A, CAP37, and PSMB2 is increased or the same when compared to a reference standard, when the reference standard is from a granulocyte suitable for treating cancer. In one embodiment expression of ANXA1 and/or PPP3CB is decreased or the same when compared to a reference standard, when the reference standard is from a granulocyte suitable for treating cancer.

In some embodiments the present invention may comprise the use of a reference standard for a granulocyte that is unsuitable for treating cancer and a reference standard for a granulocyte that is suitable for treating cancer.

A method of the invention may comprise determining the suitability of a granulocyte for treating cancer based on a comparison between a measured expression level of one or more genes of the invention and a reference standard.

In one embodiment a granulocyte is determined as being suitable for treating cancer when:
i. a measured expression level of one or more of ITGB1, CYBB, SYK, DOCK8, COMP, ATG7, SLC2A1, GZMK, CTSG, ATM, IKBKB, BCAP31, TAPBP, PERM, PLEC, ACSL1, RAC1, GM2A, CAP37, and PSMB2 is increased when compared to the reference standard when the reference standard is from a granulocyte unsuitable for treating cancer; and/or
ii. a measured expression level of ANXA1 and/or PPP3CB is decreased when compared to the reference standard, when the reference standard is from a granulocyte unsuitable for treating cancer; and/or
iii. a measured expression level of one or more of ITGB1, CYBB, SYK, DOCK8, COMP, ATG7, SLC2A1, GZMK, CTSG, ATM, IKBKB, BCAP31, TAPBP, PERM, PLEC, ACSL1, RAC1, GM2A, CAP37, and PSMB2 is increased or the same when compared to the reference standard, when the reference standard is from a granulocyte suitable for treating cancer; and/or
iv. a measured expression level of ANXA1 and/or PPP3CB is decreased or the same when compared to the reference standard, when the reference standard is from a granulocyte suitable for treating cancer.

In one embodiment a granulocyte is determined as being unsuitable for treating cancer when:
i. a measured expression level of one or more of ITGB1, CYBB, SYK, DOCK8, COMP, ATG7, SLC2A1, GZMK, CTSG, ATM, IKBKB, BCAP31, TAPBP, PERM, PLEC, ACSL1, RAC1, GM2A, CAP37, and PSMB2 is decreased or the same when compared to the reference standard, when the reference standard is from a granulocyte unsuitable for treating cancer; and/or
ii. a measured expression level of ANXA1 and/or PPP3CB is increased or the same when compared to the reference standard, when the reference standard is from a granulocyte unsuitable for treating cancer; and/or
iii. a measured expression level of one or more of ITGB1, CYBB, SYK, DOCK8, COMP, ATG7, SLC2A1, GZMK, CTSG, ATM, IKBKB, BCAP31, TAPBP, PERM, PLEC, ACSL1, RAC1, GM2A, CAP37, and PSMB2 is decreased when compared to reference standard, when the reference standard is from a granulocyte suitable for treating cancer; and/or
iv. a measured expression level of ANXA1 and/or PPP3CB is increased when compared to the reference standard, when the reference standard is from a granulocyte suitable for treating cancer.

The methods of the invention may further comprise selecting (or deselecting/discarding) a granulocyte based on the outcome of the method. In one embodiment, where a granulocyte has been determined to be suitable for treating cancer, a granulocyte may be obtained from a sample from which the tested granulocyte was originally obtained. Alternatively, or additionally a stem cell may be obtained from said sample.

Accordingly, in one aspect, there is provided an in vitro method for obtaining a granulocyte suitable for treating cancer, said method comprising obtaining a granulocyte from a sample obtainable from a donor wherein said donor produces granulocytes comprising:
a. increased expression of one or more of ITGB1, CYBB, SYK, DOCK8, COMP, ATG7, SLC2A1, GZMK, CTSG, ATM, IKBKB, BCAP31, TAPBP, PERM, PLEC, ACSL1, RAC1, GM2A, CAP37, and PSMB2 when compared to a reference standard, wherein the reference standard is from a granulocyte unsuitable for treating cancer; and/or
b. decreased expression of ANXA1 and/or PPP3CB when compared to a reference standard, wherein the reference standard is from a granulocyte unsuitable for treating cancer.

In a related aspect, there is provided an in vitro method for obtaining a stem cell suitable for treating cancer, said method comprising obtaining a stem cell from a sample obtainable from a donor wherein said donor produces granulocytes comprising:
a. increased expression of one or more of ITGB1, CYBB, SYK, DOCK8, COMP, ATG7, SLC2A1, GZMK, CTSG, ATM, IKBKB, BCAP31, TAPBP, PERM, PLEC, ACSL1, RAC1, GM2A, CAP37, and PSMB2 when compared to a reference standard, wherein the reference standard is from a granulocyte unsuitable for treating cancer; and/or
b. decreased expression of ANXA1 and/or PPP3CB when compared to a reference standard, wherein the reference standard is from a granulocyte unsuitable for treating cancer.

A method of the invention may comprise identifying whether or not a donor produces granulocytes suitable for treating cancer based on a comparison between a measured expression level of one or more genes of the invention and a reference standard.

In one embodiment a donor is identified as being a donor that produces granulocytes suitable for treating cancer when:
i. a measured expression level of one or more of ITGB1, CYBB, SYK, DOCK8, COMP, ATG7, SLC2A1, GZMK, CTSG, ATM, IKBKB, BCAP31, TAPBP, PERM, PLEC, ACSL1, RAC1, GM2A, CAP37, and PSMB2 is increased when compared to the reference standard when the reference standard is from a granulocyte unsuitable for treating cancer; and/or
ii. a measured expression level of ANXA1 and/or PPP3CB is decreased when compared to the reference standard, when the reference standard is from a granulocyte unsuitable for treating cancer; and/or
iii. a measured expression level of one or more of ITGB1, CYBB, SYK, DOCK8, COMP, ATG7, SLC2A1, GZMK, CTSG, ATM, IKBKB, BCAP31, TAPBP, PERM, PLEC, ACSL1, RAC1, GM2A, CAP37, and PSMB2 is increased or the same when compared to the reference standard, when the reference standard is from a granulocyte suitable for treating cancer; and/or
iv. a measured expression level of ANXA1 and/or PPP3CB is decreased or the same when compared to the reference standard, when the reference standard is from a granulocyte suitable for treating cancer.

In one embodiment a donor is not identified as being a donor that produces granulocytes suitable for treating cancer (or is identified as a donor that produces granulocytes that are unsuitable for treating cancer) when:
i. a measured expression level of one or more of ITGB1, CYBB, SYK, DOCK8, COMP, ATG7, SLC2A1, GZMK, CTSG, ATM, IKBKB, BCAP31, TAPBP, PERM, PLEC, ACSL1, RAC1, GM2A, CAP37, and PSMB2 is decreased or the same when compared to the reference standard, when the reference standard is from a granulocyte unsuitable for treating cancer; and/or
ii. a measured expression level of ANXA1 and/or PPP3CB is increased or the same when compared to the reference standard, when the reference standard is from a granulocyte unsuitable for treating cancer; and/or
iii. a measured expression level of one or more of ITGB1, CYBB, SYK, DOCK8, COMP, ATG7, SLC2A1, GZMK, CTSG, ATM, IKBKB, BCAP31, TAPBP, PERM, PLEC, ACSL1, RAC1, GM2A, CAP37, and PSMB2 is decreased when compared to reference standard, when the reference standard is from a granulocyte suitable for treating cancer; and/or
iv. a measured expression level of ANXA1 and/or PPP3CB is increased when compared to the reference standard, when the reference standard is from a granulocyte suitable for treating cancer.

The methods of the invention may further comprise selecting (or deselecting) a donor based on the outcome of the method. In one embodiment, where a donor has been identified as being a donor that produces granulocytes suitable for treating cancer, a granulocyte may be obtained from a sample obtainable from said donor.

In one aspect the invention provides a granulocyte obtainable by a method of the invention.

Alternatively, or additionally a stem cell may be obtained from a sample obtainable from said donor. Thus, in one aspect the invention provides a method comprising:
a. identifying a donor that produces granulocytes suitable for treating cancer according to a method of the invention; and
b. obtaining a stem cell from a sample obtainable from the donor.

Thus, in one aspect, the invention provides a stem cell obtainable by a method of the invention. The stem cell is capable of differentiating into a granulocyte for treating cancer, wherein the granulocyte comprises:
a. increased expression of one or more of ITGB1, CYBB, SYK, DOCK8, COMP, ATG7, SLC2A1, GZMK, CTSG, ATM, IKBKB, BCAP31, TAPBP, PERM, PLEC, ACSL1, RAC1, GM2A, CAP37, and PSMB2 when compared to a reference standard, wherein the reference standard is from a granulocyte unsuitable for treating cancer; and/or
b. decreased expression of ANXA1 and/or PPP3CB when compared to a reference standard, wherein the reference standard is from a granulocyte unsuitable for treating cancer.

The term “obtainable” as used herein also encompasses the term “obtained”. In one embodiment the term “obtainable” means obtained.

In a related aspect, there is provided a stem cell which is capable of differentiating into a granulocyte for treating cancer, wherein the granulocyte comprises:
a. increased expression of one or more of ITGB1, CYBB, SYK, DOCK8, COMP, ATG7, SLC2A1, GZMK, CTSG, ATM, IKBKB, BCAP31, TAPBP, PERM, PLEC, ACSL1, RAC1, GM2A, CAP37, and PSMB2 when compared to a reference standard, wherein the reference standard is from a granulocyte unsuitable for treating cancer; and/or
b. decreased expression of ANXA1 and/or PPP3CB when compared to a reference standard, wherein the reference standard is from a granulocyte unsuitable for treating cancer.

The term “stem cell” as used herein encompasses any cell that is capable of differentiating into a granulocyte (preferably a neutrophil). For example, the term “stem cell” may encompass totipotent, pluripotent, multipotent, or unipotent cells. In one embodiment the term “stem cell” encompasses a haematopoietic stem cell, as well as a precursor cell (e.g. differentiated from a haematopoietic stem cell), wherein said precursor cell is capable of differentiating into a granulocyte (preferably a neutrophil). Preferably the term “stem cell” as used herein does not encompass a human embryonic stem cell.

A stem cell may be part of a stem cell culture.

The “stem cell” may be a natural stem cell or an artificial stem cell. In one embodiment a natural stem cell may be a cell of the haematopoiesis pathway or a cell equivalent thereto. In one embodiment an artificial stem cell may be an induced pluripotent stem cell (iPSC) or a cell equivalent thereto.

In one embodiment, an iPSC is obtainable from a somatic cell, such as a somatic cell of a donor. Generation of iPSCs is a well-known technique in the art, see Yu et al (2007), Science, 318:1917-1920 the teaching of which is incorporated herein by reference.

In another embodiment, an iPSC is obtainable from a stem cell (e.g. obtainable from a donor), such as from a stem cell of the hematopoietic pathway. Preferably an iPSC is obtainable from a hematopoietic stem cell or a precursor cell described herein.

In one embodiment, a stem cell is a nuclear transfer embryonic stem cell (NT-ESC) or equivalent thereto. In one embodiment, an NT-ESC is obtainable by injecting the nucleus of a cell from the donor into an egg cell from which the original nucleus has been removed. Generation of NT-ESCs is a well-known technique in the art, see Tachibana M, Amato P, Sparman M, et al (2013), Cell, 154(2): 465-466 the teaching of which is incorporated herein by reference.

In one embodiment where a stem cell is obtained from a sample from a donor, said stem cell may be isolated from said sample. In another embodiment where a stem cell is obtained from a sample from a donor, said sample is a sample comprising stem cells or somatic cells and the stem cell is obtained by inducing pluripotency of and/or reprogramming a cell (e.g. a somatic cell) in said sample to obtain a stem cell (e.g. an iPSC).

In one embodiment the cell is reprogrammed into an induced pluripotent stem. The cell which is reprogrammed may be a hematopoietic progenitor cell, a mononuclear myeloid cell or a peripheral blood mononuclear cell using methods based on the disclosure in Ohmine et al, Stem Cell Res Ther 2011 Nov;2(6):46 and/or Rim et al, J Vis Exp 2016;(118) which are incorporated herein by reference.

In another embodiment the cell is reprogrammed into a multipotent stem cell, for example a hematopoietic stem cell, or a progenitor cell, for example a multilineage blood progenitor. The cell which is reprogrammed may be a fibroblast or a blood cell using methods based on the disclosure in Riddell et al, Cell 2014; 157(3) 549-64 and/or Szabo et al, Nature 2010; 468(7323) 521-526.

In another embodiment where a stem cell is obtained from a sample from a donor, said sample is a sample comprising stem cells or somatic cells and the stem cell is obtained by injecting the nucleus of a cell (e.g. a somatic cell) in said sample into an egg cell (e.g. from which the original nucleus has been removed) to obtain an NT-ESC.

In a preferred embodiment a stem cell is a haematopoietic stem cell. A haematopoietic stem cell may, in one embodiment, be selected on the basis of cell surface polypeptide markers, for example selected from CD34 (e.g. UniProt accession number P28906), CD59 (e.g. UniProt accession number P13987), Thy1 (e.g. UniProt accession number P04216), CD38 (e.g. UniProt accession number P28907), C-kit (e.g. UniProt accession number P10721), and lin. In one embodiment a haematopoietic stem cell comprises the cell surface polypeptide markers CD34⁺, CD59⁺, Thy1⁺, CD38^{low/-}, C-kit^{low/-}, and lin-. Preferably a haematopoietic cell expresses CD34. Antibodies to detect the presence or absence of said markers are commercially available and may be obtained from BD Biosciences Europe, ebioscience, Beckman Coulter and Pharmingen, for example.

Most preferably, a stem cell is a precursor cell (which may be referred to herein as a “granulocyte precursor cell”). In one embodiment a precursor cell is a granulocyte-committed progenitor, preferably a neutrophil-committed progenitor. A precursor cell may be one or more selected from a common myeloid progenitor cell, a myeloblast, a promyelocyte (e.g. a N. promyelocyte), a myelocyte (e.g. a N. myelocyte), a metamyelocyte (e.g. a N. metamyelocyte), a band (e.g. an N. band), or combinations thereof. Preferably, a precursor cell is a N. promyelocyte.

A stem cell of the present invention is preferably an isolated stem cell, e.g. a stem cell that has been isolated from its physiological surroundings, such as an ex vivo stem cell.

A stem cell may be differentiated into a granulocyte. A stem cell (e.g. a haematopoietic stem cell, an iPSC, or a NT-ESC) may be differentiated into another type of stem cell (e.g. a precursor cell). Differentiation may be carried out using any suitable method, such as a method based on the disclosure in Lengerke et al, Ann N Y Acad Sci, 2009 Sep; 1176:219-217, Pawlowski et al, Stem Cell Reports 2017 Apr 11;8(4):803-812, Doulatov et al, Cell Stem Cell, 2013, Oct 3; 13(4)459-470, Lieber et al, Blood, 2004 Feb 1;103(3):852-9, and/ or Choi et al, Nat. Protoc., 2011 Mar;6(3):296-313, and/or Timmins et. al. Biotechnology and bioengineering. 2009;104(4):832-40, which are incorporated herein by reference.

In one aspect, the invention provides a method for preparing a stem cell suitable for treating cancer, the method comprising:
a. identifying a donor that produces granulocytes for treating cancer according to a method of the invention; and
b. obtaining the stem cell from a sample obtainable from said donor.

In another aspect, the invention provides a method for producing a stem cell for treating cancer, the method comprising:
a. providing a stem cell obtainable from a sample from a donor wherein said donor produces granulocytes comprising:
   i. increased expression of one or more of ITGB1, CYBB, SYK, DOCK8, COMP, ATG7, SLC2A1, GZMK, CTSG, ATM, IKBKB, BCAP31, TAPBP, PERM, PLEC, ACSL1, RAC1, GM2A, CAP37, and PSMB2 when compared to a reference standard, wherein the reference standard is from a granulocyte unsuitable for treating cancer; and/or
   ii. decreased expression of ANXA1 and/or PPP3CB when compared to a reference standard, wherein the reference standard is from a granulocyte unsuitable for treating cancer; and
b. differentiating the stem cell into a different stem cell (preferably a precursor cell); and
c. optionally isolating the different stem cell (preferably the precursor cell).

In embodiments where the stem cell is a precursor cell, the different stem cell may be a different precursor cell.

In one embodiment, the sample comprises a somatic cell and obtaining the stem cell from the sample comprises reprograming the somatic cell into a stem cell.

In one aspect the invention provides a method for producing a granulocyte for treating cancer, the method comprising:
a. providing a cell; and
b. converting the cell into a granulocyte having an expression profile described herein, for example wherein:
   i. the measured expression level of one or more of GM2A, CTSG, CAP37, ITGB1, CYBB, SYK, DOCK8, COMP, ATG7, SLC2A1, GZMK, ATM, IKBKB, BCAP31, TAPBP, PERM, PLEC, ACSL1, RAC1, and PSMB2 is increased in the granulocyte when compared to a reference standard when the reference standard is from a granulocyte unsuitable for treating cancer; or
   ii. the measured expression level of ANXA1 and/or PPP3CB is decreased in the granulocyte when compared to the reference standard, when the reference standard is from a granulocyte unsuitable for treating cancer; or
   iii. the measured expression level of one or more of GM2A, CTSG, CAP37, ITGB1, CYBB, SYK, DOCK8, COMP, ATG7, SLC2A1, GZMK, ATM, IKBKB, BCAP31, TAPBP, PERM, PLEC, ACSL1, RAC1, and PSMB2 is increased or the same when compared to the reference standard, when the reference standard is from a granulocyte suitable for treating cancer; or
   iv. the measured expression level of ANXA1 and/or PPP3CB is decreased or the same when compared to the reference standard, when the reference standard is from a granulocyte suitable for treating cancer; and
c. optionally isolating the granulocyte.

In one embodiment a method for producing a granulocyte for treating cancer comprises:
a. providing a cell; and
b. converting the cell into a granulocyte having an expression profile described herein, for example wherein:
   i. the measured expression level of one or more of GM2A, CTSG, CAP37, ITGB1, CYBB, SYK, DOCK8, COMP, ATG7, SLC2A1, GZMK, ATM, IKBKB, BCAP31, TAPBP, PERM, PLEC, ACSL1, RAC1, and PSMB2 is increased in the granulocyte when compared to a reference standard when the reference standard is from a granulocyte unsuitable for treating cancer; and/or (preferably and)
   ii. the measured expression level of ANXA1 and/or PPP3CB is decreased in the granulocyte when compared to the reference standard, when the reference standard is from a granulocyte unsuitable for treating cancer; and/or (preferably and)
   iii. the measured expression level of one or more of GM2A, CTSG, CAP37, ITGB1, CYBB, SYK, DOCK8, COMP, ATG7, SLC2A1, GZMK, ATM, IKBKB, BCAP31, TAPBP, PERM, PLEC, ACSL1, RAC1, and PSMB2 is increased or the same when compared to the reference standard, when the reference standard is from a granulocyte suitable for treating cancer; and/or (preferably and)
   iv. the measured expression level of ANXA1 and/or PPP3CB is decreased or the same when compared to the reference standard, when the reference standard is from a granulocyte suitable for treating cancer; and
c. optionally isolating the granulocyte.

The cell may be a stem cell according to the invention or a somatic/differentiated cell optionally from a donor who produces granulocytes suitable for treating cancer as determined by a method of the invention. In one embodiment, converting the cell into a granulocyte comprises transdifferentiating a somatic/differentiated cell into a granulocyte using standard techniques known in the art, for example those based on Szabo et al, Nature 2010; 468(7323) 521-526.

In one embodiment converting the cell into a granulocyte comprises differentiating a stem cell into a granulocyte based on standard techniques known in the art, for example those referenced herein. For example, in one embodiment a method of differentiating a stem cell comprises admixing said stem cell with a granulocyte-macrophage colony-stimulating factor (GM-CSF), a granulocyte colony-stimulating factor (G-CSF), a growth hormone; serotonin, vitamin C, vitamin D, glutamine (Gln), arachidonic acid, AGE-albumin, an interleukin, TNF-alpha, Flt-3 ligand, thrombopoietin, foetal bovine serum (FBS), retinoic acid, lipopolysaccharide (LPS), IFN-gamma, IFN-beta or combinations thereof. In some embodiments, a method of differentiating a stem cell comprises admixing said stem cell with IFN-gamma and GM-CSF. In preferable embodiments, a method of differentiating a stem cell comprises admixing said stem cell with TNF-alpha.

In one embodiment the invention provides a method of differentiating a stem cell comprising admixing said stem cell with a granulocyte-macrophage colony-stimulating factor (GM-CSF), and a granulocyte colony-stimulating factor (G-CSF), and a growth hormone, and serotonin, and vitamin C, and vitamin D, and glutamine (Gln), and arachidonic acid, and AGE-albumin, and an interleukin, and TNF-alpha, and Flt-3 ligand, and thrombopoietin, and foetal bovine serum (FBS).

In one embodiment the invention provides a method of differentiating a stem cell comprising admixing said stem cell with a granulocyte-macrophage colony-stimulating factor (GM-CSF), and a granulocyte colony-stimulating factor (G-CSF), and a growth hormone, and serotonin, and vitamin C, and vitamin D, and glutamine (Gln), and arachidonic acid, and AGE-albumin, and an interleukin, and TNF-alpha, and Flt-3 ligand, and thrombopoietin, and foetal bovine serum (FBS), and retinoic acid, and lipopolysaccharide (LPS), and IFN-gamma, and IFN-beta.

The term “admix” as used herein means mixing one or more components together in any order, whether sequentially or simultaneously. In one embodiment “admix” means contacting a first component with a second component (e.g. a stem cell and GM-CSF).

In one embodiment differentiation of a stem cell comprises culturing said stem cell with one or more feeder cell(s). Suitably, a feeder cell may be an OP9 cell. OP9 cells (ATCC® CRL-2749™) are commercially available from the American Type Culture Collection United Kingdom (U.K.), Guernsey, Ireland, Jersey and Liechtenstein, LGC Standards, Queens Road, Teddington, Middlesex, TW11 0LY, UK. In one embodiment a stem cell may be cultured with one or more feeder cell(s) and Flt-3 ligand, thrombopoietin, fetal bovine serum (FBS), or combinations thereof.

Thus in one embodiment, a pharmaceutical composition or cell culture of the invention may further comprise a feeder cell, such as an OP9 cell.

A stem cell may be immortalised. The person skilled in the art is familiar with immortalisation techniques, which include inter alia introduction of a viral gene that deregulates the cell cycle (e.g. the adenovirus type 5 E1 gene), and artificial expression of telomerase. Immortalisation advantageously allows for the preparation of a cell line which can be stably cultured in vitro. Thus, in one aspect the invention provides an immortalised cell line obtainable (e.g. obtained) from a selected stem cell, as well as a stable stem cell culture. Suitably an immortalised cell line or stable stem cell culture is obtainable (e.g. obtained) by a method of the present invention.

The term “stable” as used in reference to a stem cell culture or cell line means that the cell culture or cell line has been modified such that it is more amenable to in vitro cell culture than an unmodified cell (i.e. a cell obtained from a donor and subjected directly to in vitro cell culture). Said “stable” cell culture or cell line is therefore capable of undergoing more rounds of replication (preferably for prolonged periods of time) when compared to an unmodified cell.

In one aspect the invention provides a method for selecting whether or not a subject is suitable for treatment with a granulocyte or a stem cell for treating cancer, the method comprising:
a. comparing a measured expression level of one or more genes by a granulocyte comprised in a sample obtainable from the subject, wherein the one or more genes are associated with suitability for treating cancer and are selected from: CTSG, CAP37, ITGB1, CYBB, SYK, DOCK8, COMP, ATG7, SLC2A1, GZMK, ATM, IKBKB, BCAP31, TAPBP, PPP3CB, ANXA1, PERM, PLEC, ACSL1, RAC1, GM2A, and PSMB2 with the expression level of the same one or more genes in a reference standard; and
b. identifying whether or not the subject is suitable for treatment with a granulocyte or a stem cell for treating cancer based on the comparison.

In one aspect the invention provides a method for selecting whether or not a subject is suitable for treatment with a granulocyte or a stem cell for treating cancer, the method comprising:
a. measuring an expression level of one or more genes by a granulocyte comprised in a sample obtainable from the subject, wherein the one or more genes are associated with suitability for treating cancer and are selected from: ITGB1, CYBB, SYK, DOCK8, COMP, ATG7, SLC2A1, GZMK, CTSG, ATM, IKBKB, BCAP31, TAPBP, PPP3CB, ANXA1, PERM, PLEC, ACSL1, RAC1, GM2A, CAP37, and PSMB2;
b. comparing the measured expression level with the expression level of the same one or more genes in a reference standard; and
c. identifying whether or not the subject is suitable for treatment with a granulocyte or a stem cell for treating cancer based on the comparison.

The foregoing method allows for the identification of subjects who have granulocytes that are unsuitable for treating cancer and who are appropriate candidates for treatment with a granulocyte or stem cell of the invention. Advantageously, patients who are most likely to respond positively to treatment can be selected, thereby allowing for more cost-effective and/or economical prescribing of the granulocyte and/or stem cell of the invention and/or avoiding selection of an incorrect patient cohort for clinical trials.

In one embodiment a subject is identified as being suitable for treatment with a granulocyte or stem cell of the invention when:
i. a measured expression level of one or more of ITGB1, CYBB, SYK, DOCK8, COMP, ATG7, SLC2A1, GZMK, CTSG, ATM, IKBKB, BCAP31, TAPBP, PERM, PLEC, ACSL1, RAC1, GM2A, CAP37, and PSMB2 is decreased or the same when compared to a reference standard, when the reference standard is from a granulocyte unsuitable for treating cancer; or
ii. a measured expression level of ANXA1 and/or PPP3CB is increased or the same when compared to a reference standard, when the reference standard is from a granulocyte unsuitable for treating cancer; or
iii. a measured expression level of one or more of ITGB1, CYBB, SYK, DOCK8, COMP, ATG7, SLC2A1, GZMK, CTSG, ATM, IKBKB, BCAP31, TAPBP, PERM, PLEC, ACSL1, RAC1, GM2A, CAP37, and PSMB2 is decreased when compared to a reference standard, when the reference standard is from a granulocyte suitable for treating cancer; or
iv. a measured expression level of ANXA1 and/or PPP3CB is increased when compared to a reference standard, when the reference standard is from a granulocyte suitable for treating cancer.

In one embodiment a subject is identified as being unsuitable for treatment with a granulocyte or stem cell of the invention when:
i. a measured expression level of one or more of ITGB1, CYBB, SYK, DOCK8, COMP, ATG7, SLC2A1, GZMK, CTSG, ATM, IKBKB, BCAP31, TAPBP, PERM, PLEC, ACSL1, RAC1, GM2A, CAP37, and PSMB2 is increased when compared to a reference standard when the reference standard is from a granulocyte unsuitable for treating cancer; or
ii. a measured expression level of ANXA1 and/or PPP3CB is decreased when compared to a reference standard, when the reference standard is from a granulocyte unsuitable for treating cancer; or
iii. a measured expression level of one or more of ITGB1, CYBB, SYK, DOCK8, COMP, ATG7, SLC2A1, GZMK, CTSG, ATM, IKBKB, BCAP31, TAPBP, PERM, PLEC, ACSL1, RAC1, GM2A, CAP37, and PSMB2 is increased or the same when compared to a reference standard, when the reference standard is from a granulocyte suitable for treating cancer; or
iv. a measured expression level of ANXA1 and/or PPP3CB is decreased or the same when compared to a reference standard, when the reference standard is from a granulocyte suitable for treating cancer

The terms “subject” and “patient” are used synonymously herein. The “subject” may be a mammal, and preferably the subject is a human subject.

In another aspect the invention provides a method for determining a subject’s risk for developing cancer, the method comprising:
a. comparing a measured expression level of one or more genes by a granulocyte comprised in a sample obtainable from the subject, wherein the one or more genes are associated with suitability for treating cancer and are selected from: CTSG, CAP37, ITGB1, CYBB, SYK, DOCK8, COMP, ATG7, SLC2A1, GZMK, ATM, IKBKB, BCAP31, TAPBP, PPP3CB, ANXA1, PERM, PLEC, ACSL1, RAC1, GM2A, and PSMB2 with the expression level of the same one or more genes in a reference standard; and
b. determining the subject’s risk for developing cancer based on the comparison.

In a related aspect there is provided a method for determining a subject’s risk for developing cancer, the method comprising:
a. measuring an expression level of one or more genes by a granulocyte comprised in a sample obtainable from the subject, wherein the one or more genes are associated with suitability for treating cancer and are selected from: ITGB1, CYBB, SYK, DOCK8, COMP, ATG7, SLC2A1, GZMK, CTSG, ATM, IKBKB, BCAP31, TAPBP, PPP3CB, ANXA1, PERM, PLEC, ACSL1, RAC1, GM2A, CAP37, and PSMB2;
b. comparing the measured expression level with the expression level of the same one or more genes in a reference standard; and
c. determining the subject’s risk for developing cancer based on the comparison.

In one embodiment a subject is determined as being at risk of developing cancer when:
i. a measured expression level of one or more of ITGB1, CYBB, SYK, DOCK8, COMP, ATG7, SLC2A1, GZMK, CTSG, ATM, IKBKB, BCAP31, TAPBP, PERM, PLEC, ACSL1, RAC1, GM2A, CAP37, and PSMB2 is decreased or the same when compared to a reference standard, when the reference standard is from a granulocyte unsuitable for treating cancer; or
ii. a measured expression level of ANXA1 and/or PPP3CB is increased or the same when compared to a reference standard, when the reference standard is from a granulocyte unsuitable for treating cancer; or
iii. a measured expression level of one or more of ITGB1, CYBB, SYK, DOCK8, COMP, ATG7, SLC2A1, GZMK, CTSG, ATM, IKBKB, BCAP31, TAPBP, PERM, PLEC, ACSL1, RAC1, GM2A, CAP37, and PSMB2 is decreased when compared to a reference standard, when the reference standard is from a granulocyte suitable for treating cancer
iv. a measured expression level of ANXA1 and/or PPP3CB is increased when compared to a reference standard, when the reference standard is from a granulocyte suitable for treating cancer.

In one embodiment a subject is determined as not at risk of developing cancer when:
i. a measured expression level of one or more of ITGB1, CYBB, SYK, DOCK8, COMP, ATG7, SLC2A1, GZMK, CTSG, ATM, IKBKB, BCAP31, TAPBP, PERM, PLEC, ACSL1, RAC1, GM2A, CAP37, and PSMB2 is increased when compared to a reference standard when the reference standard is from a granulocyte unsuitable for treating cancer; or
ii. a measured expression level of ANXA1 and/or PPP3CB is decreased when compared to a reference standard, when the reference standard is from a granulocyte unsuitable for treating cancer; or
iii. a measured expression level of one or more of ITGB1, CYBB, SYK, DOCK8, COMP, ATG7, SLC2A1, GZMK, CTSG, ATM, IKBKB, BCAP31, TAPBP, PERM, PLEC, ACSL1, RAC1, GM2A, CAP37, and PSMB2 is increased or the same when compared to a reference standard, when the reference standard is from a granulocyte suitable for treating cancer; or
iv. a measured expression level of ANXA 1 and/or PPP3CB is decreased or the same when compared to a reference standard, when the reference standard is from a granulocyte suitable for treating cancer

The term “granulocyte” encompasses the following cell types: neutrophils, basophils, and eosinophils. Preferably the granulocyte is a neutrophil. A granulocyte may express the cell surface polypeptide markers CD11b (e.g. UniProt accession number P11215) and CD15. A granulocyte may also produce reactive oxygen species (O₂⁻). Preferably the granulocyte CD11b^{high}. Alternatively or additionally, the granulocyte may have a higher density than granulocytes unsuitable for treating cancer, and/or a positive cell surface charge (e.g. a net cell charge).

A granulocyte of the present invention is preferably an isolated granulocyte, e.g. a granulocyte that has been isolated from its physiological surroundings, such as an ex vivo granulocyte.

In some embodiments the granulocyte is obtainable from a sample obtainable from a donor. In another embodiment a granulocyte may be an engineered granulocyte. Such a granulocyte may be produced by a method comprising:
a. providing a granulocyte; and
b. engineering the granulocyte to:
   i. increase expression of one or more genes selected from: ITGB1, CYBB, SYK, DOCK8, COMP, ATG7, SLC2A1, GZMK, CTSG, ATM, IKBKB, BCAP31, TAPBP, PERM, PLEC, ACSL1, RAC1, GM2A, CAP37, and PSMB2; and/or
   ii. decrease expression of ANXA1 and/or PPP3CB;
thereby producing the engineered granulocyte, wherein the engineered granulocyte is suitable for treating cancer. In some embodiments the engineered granulocyte may be used as a reference standard in a method of the invention (i.e. as a reference standard from a granulocyte suitable for treating cancer).

The granulocyte provided for use in the method is preferably a granulocyte that is unsuitable for treating cancer. Thus, in some embodiments a method of the invention converts a cell that is unsuitable for treating cancer into a granulocyte that is suitable for treating cancer. Said granulocyte may be identified by a method described herein, obtained from a donor identified by a method described herein (e.g. from a subject suitable for treatment with a granulocyte or stem cell of the invention), and/or from a subject identified as being at risk of developing cancer.

In a related aspect the invention provides a method for producing an engineered stem cell, the method comprising:
a. providing a stem cell; and
b. engineering the stem cell to:
   i. increase expression of one or more genes selected from: ITGB1, CYBB, SYK, DOCK8, COMP, ATG7, SLC2A1, GZMK, CTSG, ATM, IKBKB, BCAP31, TAPBP, PERM, PLEC, ACSL1, RAC1, GM2A, CAP37, and PSMB2; and/or
   ii. decrease expression of ANXA1 and/or PPP3CB;
thereby producing the engineered stem cell, wherein the engineered stem cell is suitable for treating cancer. In some embodiments the engineered stem cell may be used as a reference standard in a method of the invention (i.e. as a reference standard from a stem cell suitable for treating cancer).

In a related aspect the invention provides a method for producing an engineered stem cell, the method comprising:
a. providing a stem cell; and
b. engineering the stem cell such that it is capable of differentiating into a granulocyte having:
   i. increased expression of one or more genes selected from: ITGB1, CYBB, SYK, DOCK8, COMP, ATG7, SLC2A1, GZMK, CTSG, ATM, IKBKB, BCAP31, TAPBP, PERM, PLEC, ACSL1, RAC1, GM2A, CAP37, and PSMB2; and/or
   ii. decreased expression of ANXA1 and/or PPP3CB;
thereby producing the engineered stem cell, wherein the engineered stem cell is suitable for treating cancer. In some embodiments the engineered stem cell may be used as a reference standard in a method of the invention (i.e. as a reference standard from a stem cell suitable for treating cancer).

The stem cell provided for use in the method is preferably a stem cell that is unsuitable for treating cancer. Thus, in some embodiments a method of the invention converts a stem cell that is unsuitable for treating cancer into a stem cell that is suitable for treating cancer. Said stem cell may be identified by a method described herein, obtained from a donor identified by a method described herein (e.g. from a subject suitable for treatment with a granulocyte or stem cell of the invention), and/or from a subject identified as being at risk of developing cancer.

Engineering a stem cell or granulocyte may be carried out in vivo, for example in one aspect the invention comprises engineering a stem cell or granulocyte in a subject, preferably engineering a stem cell in a subject. In one embodiment the invention may comprise engineering a stem cell or granulocyte in situ in a subject (e.g. in the bone marrow of a subject). Suitably, said subject may be a subject that produces stem cells or granulocytes that are unsuitable for treating cancer, a subject that is suitable for treatment with a granulocyte or stem cell of the invention, a subject that is at risk of developing cancer, or combinations thereof.

The engineering may be carried out using any means known to the person skilled in the art. In one embodiment expression may be increased or decreased using genome editing. In one embodiment expression may be increased or decreased using CRISPR (e.g. the DNA-snipping CRISPR-associated endonuclease Cas9 genome-editing system), TALENS, Zinc finger proteins, adenoviruses (AV), retroviruses, vectors (e.g. inducible and/or over-expressible vectors), transgene insertion, cisgene over- or under-expression, silencing, or epigenetic modulation of promoter regions through histone deacetylase (HDAC) inhibitors, or combinations thereof. The expression of genes associated with suitability for treating cancer can be modulated in stem cells (e.g. myeloblasts) using methods of culturing (adapted from Gupta D, Shah HP, Malu K, Berliner N, Gaines P. Differentiation and characterization of myeloid cells. Curr Protoc Immunol. 2014;104:Unit 22F 25.), and in any suitable cells by using CRISPR methods (adapted from N.E. Sanjana, O. Shalem, F. Zhang Improved vectors and genome-wide libraries for CRISPR screening Nat. Methods, 11 (2014), pp. 783-784), TALEN systems (adapted from A.A. Nemudryi, K.R. Valetdinova, S.P. Medvedev, and S.M. Zakian TALEN and CRISPR/Cas genome editing systems: tools of discovery. Acta Naturae. 2014; 6(3); 19-40), and Zinc finger proteins (adapted from M.C. Keightley et al. The Pu.1 target gene Zbtb11 regulates neutrophil development through its integrase-like HHCC zinc finger. Nat Commun. 2017;8;14911) to generate cells with key genes knocked-in or knocked-out. Where the cell is a stem cell, said cell can be differentiated to produce granulocytes. Briefly, by using lentiviral transduction of single guide CRISPR-Cas9 vectors, pre-validated CRISPR (guide) gRNA sequences to genes associated with suitability for treating cancer in the lentiviral vector lentiCRISPRv2 can be ordered from GenScript or AddGene. CRISPR knockout experiments may use targeting sequences within exons, whereas CRISPR activation or repression experiments may use targets within promoters. ANXA1 for instance, can be knocked-out of a cell to improve cancer killing activity using a pre-validated gRNA targeting its exon. Lentiviral vectors may be prepared and suitable cells transduced (according to previously published protocols (Satchwell TJ, Hawley BR, Bell AJ, Ribeiro ML, Toye AM. The cytoskeletal binding domain of band 3 is required for multiprotein complex formation and retention during erythropoiesis. Haematologica 2015;100(1):133-142.). Verification of CRISPR on- and off-target effects can be confirmed via whole genome sequencing by comparing the genomic differences between the unedited control and the modified samples. Modified myeloblasts may then be differentiated and identified using the methods of Gupta and colleagues (2014). In one embodiment said approaches may be applied to granulocytes or stem cells.

In some embodiments the engineering may be modulation of one or more cytokines driving lineage in stem cells and/or genes associated with suitability for treating cancer.

In one aspect of the invention, there is provided a granulocyte (or engineered granulocyte) for treating cancer, wherein the granulocyte comprises:
a. increased expression of one or more of ITGB1, CYBB, SYK, DOCK8, COMP, ATG7, SLC2A1, GZMK, CTSG, ATM, IKBKB, BCAP31, TAPBP, PERM, PLEC, ACSL1, RAC1, GM2A, CAP37, and PSMB2 when compared to a reference standard, wherein the reference standard is from a granulocyte unsuitable for treating cancer; and/or
b. decreased expression of ANXA1 and/or PPP3CB when compared to a reference standard, wherein the reference standard is from a granulocyte unsuitable for treating cancer.

In one embodiment a granulocyte comprises:
a. increased expression of one or more of ITGB1, CYBB, SYK, DOCK8, COMP, ATG7, SLC2A1, GZMK, CTSG, ATM, IKBKB, BCAP31, TAPBP, PERM, PLEC, ACSL1, RAC1, GM2A, CAP37, and PSMB2 when compared to a reference standard, wherein the reference standard is from a granulocyte unsuitable for treating cancer; and
b. decreased expression of ANXA1 and/or PPP3CB when compared to a reference standard, wherein the reference standard is from a granulocyte unsuitable for treating cancer.

In a particularly preferred embodiment a granulocyte of the invention has a positively charged cell surface (or a more positively charged cell surface when compared to a granulocyte that is unsuitable for treating cancer).

The skilled person would appreciate that the granulocytes or stem cells of the invention do not need to be activated ex vivo to be suitable for treating cancer. To the extent that the granulocytes or stem cells of the invention are activated ex vivo, the skilled person would appreciate that any activation would further increase the cancer killing activity of the granulocytes or stem cells. Accordingly, in some embodiments, the granulocyte or stem cell for treating cancer has not been activated ex vivo. In some embodiments, the granulocyte or stem cell for treating cancer has not been activated ex vivo with a chemokine or cytokine. For example, in some embodiments, the granulocyte or stem cell for treating cancer has not been activated ex vivo with TGFβ, CCL2, CCL3, CCL5, CXC1, CXC12 and/or CXC16. In preferred embodiments, the granulocyte or stem cell for treating cancer has not been activated ex vivo with CCL2. In preferred embodiments, the granulocyte or stem cell for treating cancer has not been activated ex vivo with TGFβ. In some embodiments, the granulocyte or stem cell for treating cancer has not been activated with a granulocyte-macrophage colony-stimulating factor (GM-CSF), a granulocyte colony-stimulating factor (G-CSF), a growth hormone; serotonin, vitamin C, vitamin D, glutamine (Gln), arachidonic acid, AGE-albumin, an interleukin, TNF-alpha, Flt-3 ligand, thrombopoietin, foetal bovine serum (FBS), retinoic acid, lipopolysaccharide (LPS), IFN-gamma, IFN-beta or combinations thereof. Thus, in some embodiments, the granulocyte or stem cell for treating cancer is an unactivated granulocyte or stem cell.

Accordingly, in some embodiments of a method of the invention, the method does not comprise activating a granulocyte or stem cell for treating cancer ex vivo. For example, in some embodiments, the method does not comprise activating the granulocyte or stem cell for treating cancer. In some embodiments, the method does not comprise activating the granulocyte or stem cell for treating cancer ex vivo with a chemokine or cytokine. For example, in some embodiments, the method does not comprise activating the granulocyte or stem cell for treating cancer ex vivo with TGFβ, CCL2, CCL3, CCL5, CXC1, CXC12 and/or CXC16. In preferred embodiments, the method does not comprise activating the granulocyte or stem cell for treating cancer ex vivo with CCL2. In preferred embodiments, the method does not comprise activating the granulocyte or stem cell for treating cancer ex vivo with TGFβ. In some embodiments, the method does not comprise activating the granulocyte or stem cell for treating cancer ex vivo with a granulocyte-macrophage colony-stimulating factor (GM-CSF), a granulocyte colony-stimulating factor (G-CSF), a growth hormone; serotonin, vitamin C, vitamin D, glutamine (Gln), arachidonic acid, AGE-albumin, an interleukin, TNF-alpha, Flt-3 ligand, thrombopoietin, foetal bovine serum (FBS), retinoic acid, lipopolysaccharide (LPS), IFN-gamma, IFN-beta or combinations thereof.

In alternative embodiments, the granulocyte or stem cell for treating cancer has been activated ex vivo. In some embodiments, the granulocyte or stem cell for treating cancer has been activated ex vivo with a chemokine or cytokine. For example, in some embodiments, the granulocyte or stem cell for treating cancer has been activated ex vivo with TGFβ, CCL2, CCL3, CCL5, CXC1, CXC12 and/or CXC16. In preferred embodiments, the granulocyte or stem cell for treating cancer has been activated ex vivo with CCL2. In preferred embodiments, the granulocyte or stem cell for treating cancer has been activated ex vivo with TGFβ. In some embodiments, the granulocyte or stem cell has been activated with a granulocyte-macrophage colony-stimulating factor (GM-CSF), a granulocyte colony-stimulating factor (G-CSF), a growth hormone; serotonin, vitamin C, vitamin D, glutamine (Gln), arachidonic acid, AGE-albumin, an interleukin, TNF-alpha, Flt-3 ligand, thrombopoietin, foetal bovine serum (FBS), retinoic acid, lipopolysaccharide (LPS), IFN-gamma, IFN-beta or combinations thereof. In some embodiments, the granulocyte or stem cell for treating cancer has been activated with IFN-gamma and GM-CSF. In preferred embodiments, the granulocyte or stem cell for treating cancer has been activated with TNF-alpha. Thus, in some embodiments, the granulocyte or stem cell for treating cancer is an activated granulocyte or stem cell.

Accordingly, in some embodiments of a method of the invention, the method comprises activating a granulocyte or stem cell for treating cancer ex vivo. For example, in some embodiments, the method comprises activating the granulocyte or stem cell for treating cancer. In some embodiments, the method does comprises activating the granulocyte or stem cell for treating cancer ex vivo with a chemokine or cytokine. For example, in some embodiments, the method comprises activating the granulocyte or stem cell for treating cancer ex vivo with TGFβ, CCL2, CCL3, CCL5, CXC1, CXC12 and/or CXC16. In some embodiments, the method comprises activating the granulocyte or stem cell for treating cancer ex vivo with CCL2. In some embodiments, the method comprises activating the granulocyte or stem cell for treating cancer ex vivo with TGFβ. In some embodiments, the method comprises activating the granulocyte or stem cell for treating cancer ex vivo with a granulocyte-macrophage colony-stimulating factor (GM-CSF), a granulocyte colony-stimulating factor (G-CSF), a growth hormone; serotonin, vitamin C, vitamin D, glutamine (Gln), arachidonic acid, AGE-albumin, an interleukin, TNF-alpha, Flt-3 ligand, thrombopoietin, foetal bovine serum (FBS), retinoic acid, lipopolysaccharide (LPS), IFN-gamma, IFN-beta or combinations thereof.

The present invention may further comprise the validation of a granulocyte or stem cell’s suitability for treating cancer by a different means, e.g. by way of cell surface charge and/or by way of a functional assay.

In one embodiment granulocyte cell surface charge correlates with suitability for treating cancer, with granulocytes (e.g. neutrophils) that are more positively charged (or less negatively charged) being suitable for treating cancer and/or more efficacious in treating cancer. The level of cell surface charge may be determined when compared to a reference standard, preferably wherein the reference standard is from a granulocyte that is unsuitable for treating cancer. In one embodiment a stem cell may be considered as suitable for treating cancer if it is capable of differentiating into a granulocyte having a more positively charged (or less negatively charged) cell surface. A cell surface charge can be determined using any suitable technique known in the art. In one embodiment the cell surface charge is determined using electrophoresis. An electrophoretic mobility assay may be one described in “Cell Electrophoresis” edited by Johann Bauer (ISBN 0-8493-8918-6 published by CRC Press, Inc.) the teaching of which is incorporated herein in its entirety. In another embodiment cell surface charge can be determined using negatively and/or positively charged means. In one embodiment, a granulocyte has a positive cell surface charge when it can be bound by a negatively charged means, and not a positively charged means. In one embodiment, a granulocyte has a negative cell surface charge when it can be bound by a positively charged means, and not a negatively charged means. Such negatively and/or positively charged means may also be used to measure the concentration of a granulocyte cell in a sample. A positively charged means may be a positively charged particle, nanoprobe or nanoparticle, or a cation exchange media. Suitable nanoparticles may be prepared by conjugating superparamagnetic Iron(II,III) oxide (Fe₃O₄) nanoparticles (NPs) with (3-Aminopropyl)triethoxysilane (APTES) to form a thin layer of Silicon dioxide (SiO₂) shell on the NPs’ surface upon reaction with Tetraethyl orthosilicate (TEOS) and ammonium hydroxide (NH₄OH). Fluorescein isothiocyanates (FITCs) may be embedded in the SiO₂ shell, thus exposing the Si-linked hydroxyl groups (SiO₂—OH) and creating the negative surface charge. Branched poly(ethylene imine) (PEI) molecules may be used to not only to cover the SiO₂—OH groups in a non-covalently manner but also to expose the additional amine groups that carry the positive charges. Thus, in one embodiment a negatively charged nanoparticle is prepared by conjugating Fe₃O₄ nanoparticles with APTES to form a thin layer of SiO₂ shell on the nanoparticle surface upon reaction with Tetraethyl orthosilicate (TEOS) and ammonium hydroxide (NH₄OH), and embedding a FITC in the SiO₂ shell, thus exposing the SiO₂—OH groups (creating the negative surface charge). In another embodiment, a positively charged nanoparticle is prepared by contacting a negatively charged nanoparticle (as described herein) with a PEI molecule (e.g. to expose additional amine groups that carry a positive charge). In one embodiment, the negatively charged means (e.g. nanoparticle) may have a negative surface charge of at least -5 mV, -10 mV, -20 mV, -30 mV, or -40 mV. Preferably, the negatively charged means (e.g. nanoparticle) has may have a negative surface charge of at least -35 mV. In one embodiment, the positively charged means (e.g. nanoparticle) may have a positive surface charge of at least +5 mV, +10 mV, +20 mV, +30 mV, or +40 mV. Preferably, the positively charged means (e.g. nanoparticle) has may have a positive surface charge of at least +35 mV. The surface charge of said positively or negatively charged means (e.g. nanoparticle) may refer to the surface zeta potential of the positively or negatively charged means (e.g. nanoparticle). The surface zeta potential may be measured with a Dynamic light scattering particle size analyser (e.g. the Zetasizer Nano-ZS90, Malvern, UK). In one aspect the present invention involves isolating granulocytes comprising a (more) positive cell surface charge by way of said charge. For example, said cells may be isolated using a negatively charged means, such as a negatively charged particle, nanoprobe or nanoparticle, or an anion exchange media. Such techniques may be used to measure the cell surface charge of granulocytes or the concentration of granulocytes having a positive cell surface charge in the foregoing embodiments. The cells may be isolated from negatively charged, neutrally charged, or less positively charged granulocytes. In one embodiment, a positively or negatively charged means (e.g. nanoparticle) may be detectable by fluorescence. In another embodiment, a positively or negatively charged means (e.g. nanoparticle) may be capable of being captured by way of magnetism, thus allowing isolation of a cell that interacts with said means.

A functional assay for validating the suitability of a granulocyte or stem cell for treating cancer may comprise:
a. contacting cancer cells with a granulocyte to form a test sample;
b. incubating the test sample; and
c. measuring the % of cancer cells killed in the test sample.

To validate a stem cell’s suitability, said stem cell may be differentiated into a granulocyte which is employed in the above-mentioned assay.

In one embodiment a granulocyte or stem cell is validated according to the assay when the granulocyte kills at least 70% of the cancer cells in the test sample. Preferably, a granulocyte or stem cell is validated according to the assay when the granulocyte kills at least 80% or 90% of the cancer cells in the test sample.

The cancer cell for use in an assay may be one or more selected from a pancreatic cancer cell line, a liver cancer cell line, an oesophageal cancer cell line, a stomach cancer cell line, a cervical cancer cell line, an ovarian cancer cell line, a lung cancer cell line, a bladder cancer cell line, a kidney cancer cell line, a brain cancer cell line, a prostate cancer cell line, a myeloma cancer cell line, a non-Hodgkin’s lymphoma (NHL) cell line, a larynx cancer cell line, a uterine cancer cell line, or a breast cancer cell line. Suitable cell lines are available commercially from the American Type Culture Collection United Kingdom (U.K.), Guernsey, Ireland, Jersey and Liechtenstein, LGC Standards, Queens Road, Teddington, Middlesex, TW11 0LY, UK. For example, a pancreatic cell line may be one or more of Capan-2, ATCC HTB-80; Panc 10.05, ATCC CRL-2547; CFPAC-1, ATCC CRL-1918; HPAF-II, ATCC CRL-1997; SW 1990, ATCC CRL-2172; BxPC-3, ATCC CRL-1687; AsPC-1, ATCC CRL-1682; ATCC® TCP-1026™; SW1990, ATCC CRL-2172; SU.86.86, ATCC CRL-1837; BXPC-3, ATCC CRL-1687; Panc 10.05, ATCC CRL-2547; MIA-PaCa-2, ATCC CRL-1420; PANC-1, ATCC CRL-1469; or ATCC® TCP-2060™. Preferably the cancer cell line is pancreatic cancer cell line, such as PANC-1. In one embodiment the cancer cell line is a cervical cancer cell line, such as a HeLa cell.

The incubation step may be carried out for between 1 hour and 100 hours. Suitably, the incubation step may be carried out for between 5 hours and 75 hours, for example between 10 hours and 20 hours. The incubation step may be carried out for between 6 hours to 6 days. Suitably, the incubation step may be carried out for between 6 hours and 2 days, for example for between 12 hours to 36 hours, such as between 16 to 24 hours. In one embodiment the incubation step is carried out for 24 hours. In another embodiment the incubation step is carried out for 48 hours. The incubation step may be carried out at any temperature suitable for cell growth and viability, for example at a temperature between 35° C. to 42° C., suitably at 37 or 39° C. Preferably the incubation step is carried out at 37 or 39° C. for 24 hours. Preferably the incubation step is carried out for 16-24 hours at 30-40° C. (e.g. 37° C.).

The % of cancer cells killed can be measured by reference to the total number of starting cancer cells. The number of cancer cells killed can be measured using any suitable means, for example by viability staining (e.g. trypan blue staining), and microscopy, or using other automated means, for example by cell electronic sensing equipment, such as the RT-CES™ system available from ACEA Biosciences, Inc. (11585 Sorrento Valley Rd., Suite 103, San Diego, CA 92121, USA). In some embodiments the % of cancer cells killed may be determined within 24 hours (e.g. of incubating a cancer cell line and a granulocyte). The % of cancer cells killed is preferably the maximum number of cancer cells killed when carrying out a method of the invention.

The number of cancer cells killed can be also be measured using the ACEA Biosciences xCELLigence RTCA DP Analyzer system®. The xCELLigence System is a real-time cell analyser, allowing for label-free and dynamic monitoring of cellular phenotypic changes continuously by measuring electrical impedance. Such measurements may be carried out as detailed in Example 11. Said System is commercially available from ACEA Biosciences 6779 Mesa Ridge Road #100, San Diego, CA 92121 USA.

A ratio of at least 1:1, 5:1 or 10:1 of granulocytes to cancer cells may be used. Preferably a 5:1 ratio of granulocytes to cancer cells is used. More preferably a 10:1 ratio of granulocytes to cancer cells is used.

A granulocyte or stem cell described herein may be part of a cell culture (e.g. an in vitro cell culture). Accordingly, in one aspect, there is provided an in vitro culture of granulocytes of the invention. In a related aspect, there is provided an in vitro culture of stem cells of the invention.

A granulocyte or stem cell of the invention may be subjected to one or more further processing steps, such as cryogenic freezing. The further processing step may include admixing said granulocyte or stem cell with a preservation medium, for example a cryogenic preservation medium.

In one aspect the invention provides a composition for treating cancer, the composition comprising granulocytes: wherein at least 90% of the granulocytes comprised in the composition have:
a. increased expression of one or more of ITGB1, CYBB, SYK, DOCK8, COMP, ATG7, SLC2A1, GZMK, CTSG, ATM, IKBKB, BCAP31, TAPBP, PERM, PLEC, ACSL1, RAC1, GM2A, CAP37, and PSMB2 when compared to a reference standard, wherein the reference standard is from a granulocyte unsuitable for treating cancer; and/or
b. decreased expression of ANXA1 and/or PPP3CB when compared to a reference standard, wherein the reference standard is from a granulocyte unsuitable for treating cancer.

In one aspect the invention provides a composition for treating cancer, the composition comprising granulocytes: wherein at least 95% of the granulocytes comprised in the composition have:
a. increased expression of one or more of ITGB1, CYBB, SYK, DOCK8, COMP, ATG7, SLC2A1, GZMK, CTSG, ATM, IKBKB, BCAP31, TAPBP, PERM, PLEC, ACSL1, RAC1, GM2A, CAP37, and PSMB2 when compared to a reference standard, wherein the reference standard is from a granulocyte unsuitable for treating cancer; and/or
b. decreased expression of ANXA1 and/or PPP3CB when compared to a reference standard, wherein the reference standard is from a granulocyte unsuitable for treating cancer.

In one aspect the invention provides a composition for treating cancer, the composition comprising granulocytes: wherein at least 99% of the granulocytes comprised in the composition have:
a. increased expression of one or more of ITGB1, CYBB, SYK, DOCK8, COMP, ATG7, SLC2A1, GZMK, CTSG, ATM, IKBKB, BCAP31, TAPBP, PERM, PLEC, ACSL1, RAC1, GM2A, CAP37, and PSMB2 when compared to a reference standard, wherein the reference standard is from a granulocyte unsuitable for treating cancer; and/or
b. decreased expression of ANXA1 and/or PPP3CB when compared to a reference standard, wherein the reference standard is from a granulocyte unsuitable for treating cancer.

In one aspect the invention provides a composition for treating cancer, the composition comprising granulocytes: wherein at least 100% of the granulocytes comprised in the composition have:
a. increased expression of one or more of ITGB1, CYBB, SYK, DOCK8, COMP, ATG7, SLC2A1, GZMK, CTSG, ATM, IKBKB, BCAP31, TAPBP, PERM, PLEC, ACSL1, RAC1, GM2A, CAP37, and PSMB2 when compared to a reference standard, wherein the reference standard is from a granulocyte unsuitable for treating cancer; and/or
b. decreased expression of ANXA1 and/or PPP3CB when compared to a reference standard, wherein the reference standard is from a granulocyte unsuitable for treating cancer.

In one embodiment at least 90%, 95%, 99% or 100% of the granulocytes comprised in the composition have:
a. increased expression of one or more of ITGB1, CYBB, SYK, DOCK8, COMP, ATG7, SLC2A1, GZMK, CTSG, ATM, IKBKB, BCAP31, TAPBP, PERM, PLEC, ACSL1, RAC1, GM2A, CAP37, and PSMB2 when compared to a reference standard, wherein the reference standard is from a granulocyte unsuitable for treating cancer; and
b. decreased expression of ANXA1 and/or PPP3CB when compared to a reference standard, wherein the reference standard is from a granulocyte unsuitable for treating cancer.

Advantageously, the compositions of the invention contain a substantially homogeneous population of granulocytes that are suitable for treating cancer.

The invention also provides a method for isolating granulocytes suitable for treating cancer based on the expression of one or more genes of the invention. Such methods may provide a substantially homogeneous population of granulocytes that are suitable for treating cancer. In one embodiment, granulocytes for treating cancer are isolated using the expression of one or more cell-surface expressed polypeptides selected from: ATG7, CYBB, DOCK8, CTSG, S100A9, COMP, S100A8, CTSG, SYK, ITGB1, SLC2A1, GZMK, ANXA1, RAC1, and CAP37, preferably one or more selected from: ATG7, S100A9, COMP, S100A8, CTSG, SYK, ITGB1, SLC2A1, GZMK, ANXA1, RAC1, and CAP37. The isolation may be performed using any suitable technique. In one embodiment a method for isolating granulocytes comprises the use of a binding means that binds to a polypeptide of the invention. Preferably the binding means is an antibody. Antibodies to detect the presence or absence of polypeptides of the invention are commercially available: anti-CYBB antibody (Cat# M03328, BosterBio), anti-DOCK8 antibody (Ab227529, AbCam), anti-ATG7 antibody (Cat# HPA007639, Atlas Antibodies), anti-S100A9 antibody (HPA004193, Atlas Antibodies), anti-ACSL1 antibody (Cat# HPA011964, Atlas Antibodies), anti-ATM antibody (Cat# HPA067142, Atlas Antibodies), anti-COMP antibody (Cat# AF3134, R&D Systems), anti-TAPBP antibody (Cat# HPA007066, Atlas Antibodies), anti-S100A8 antibody (Cat# AF4570, R&D Systems), anti-PLEC antibody (Cat# HPA029906, Atlas Antibodies), anti-BCAP31 antibody (Cat# HPA003906, Atlas Antibodies), anti-CTSG antibody (Cat# C35667, Sab Biotech), anti-SYK antibody (Cat# Ab40781, AbCam), anti-ITGB1 antibody (Cat# P260111, Sino Biological), anti-PSMB2 antibody (Cat# HPA026322, Atlas Antibodies), anti-GM2A antibody (Cat# HPA008063, Atlas Antibodies), anti-SLC2A1 antibody (Cat# HPA031345, Atlas Antibodies), anti-GZMK antibody (Cat# HPA063181, Atlas Antibodies), anti-IKBKB antibody (Cat# HPA001249, Atlas Antibodies), anti-PPP3CB antibody (Cat# HPA008233, Atlas Antibodies), anti-ANXA1 antibody (Cat# HPA011271, Atlas Antibodies), anti-PERM antibody (Cat# HPA021147, Atlas Antibodies), anti-RAC1 antibody (Cat# HPA047820, Atlas Antibodies), and anti-CAP37 antibody (Cat# HPA055851, Atlas Antibodies). The method may comprise the use of flow cytometric techniques, preferably fluorescence activated cell sorting (FACS), e.g. together with appropriate ‘gating’. Flow cytometric techniques may be particularly suitable when the method employs the use of a binding means coupled to a detectable label, such as a fluorophore.

In one aspect there is provided a method for isolating a granulocyte for treating cancer, the method comprising:
a) contacting a sample of granulocytes with a binding means, wherein the binding means binds to one or more polypeptides selected from CTSG, CAP37, ITGB1, CYBB, SYK, DOCK8, COMP, ATG7, SLC2A1, GZMK, ATM, IKBKB, BCAP31, TAPBP, PPP3CB, ANXA1, PERM, PLEC, ACSL1, RAC1, GM2A, and PSMB2; and
b) isolating the granulocyte based on the presence or absence of binding between the binding means and the one or more polypeptides.

Binding may be determined to be present when the amount of binding is statistically significant (e.g. when compared to a ‘background’ control). Binding may be determined to be absent when the amount of binding is statistically insignificant (e.g. when compared to a ‘background’ control). Preferably, binding is determined to be absent when there is no binding whatsoever.

In one embodiment the invention comprises detecting the presence of one or more polypeptides selected from CTSG, CAP37, ITGB1, CYBB, SYK, DOCK8, COMP, ATG7, SLC2A1, GZMK, ATM, IKBKB, BCAP31, TAPBP, PERM, PLEC, ACSL1, RAC1, GM2A, and PSMB2. When said one or more polypeptides are detected (i.e. where there is binding between the binding means and the polypeptide) the granulocyte may be isolated. Suitably, said isolated granulocyte may be a granulocyte for treating cancer.

In another embodiment the invention may comprise detecting the absence of ANXA1 and/or PPP3CB (e.g. not detecting ANXA1 and/or PPP3CB). When said one or more polypeptides are not detected (i.e. where there is an absence of binding between the binding means and the polypeptide) the granulocyte may be isolated. Suitably, said isolated granulocyte may be a granulocyte for treating cancer.

Preferably, the invention comprises detecting:
the presence of one or more polypeptides selected from CTSG, CAP37, ITGB1, CYBB, SYK, DOCK8, COMP, ATG7, SLC2A1, GZMK, ATM, IKBKB, BCAP31, TAPBP, PERM, PLEC, ACSL1, RAC1, GM2A, and PSMB2; and
the invention absence of ANXA1 and/or PPP3CB. In one embodiment, when said polypeptides are detected or not detected (as indicated), the granulocyte is isolated.

In one embodiment a method of isolating a granulocyte comprises the use of an immobilised binding means (e.g. a binding means conjugated to a bead, such as a magnetic bead, or chromatographic resin) to isolate a granulocyte of the invention. Such methods may be immuno-affinity methods.

The method may comprise quantifying the amount of binding between the binding means and the one or more polypeptides or between the binding means and the granulocyte. The method may comprise isolating a granulocyte for treating cancer based on the quantified amount of binding.

In one embodiment a granulocyte for treating cancer is isolated when there is a high level of binding between a binding means and one or more polypeptides selected from CTSG, CAP37, ITGB1, CYBB, SYK, DOCK8, COMP, ATG7, SLC2A1, GZMK, ATM, IKBKB, BCAP31, TAPBP, PERM, PLEC, ACSL1, RAC1, GM2A, and PSMB2.

In one embodiment a granulocyte for treating cancer is isolated when there is a low level of binding between a binding means and ANXA1 and/or PPP3CB.

Preferably, a granulocyte for treating cancer is isolated when there is:
a high level of binding between a binding means and one or more polypeptides selected from CTSG, CAP37, ITGB1, CYBB, SYK, DOCK8, COMP, ATG7, SLC2A1, GZMK, ATM, IKBKB, BCAP31, TAPBP, PERM, PLEC, ACSL1, RAC1, GM2A, and PSMB2; and
a low level of binding between a binding means and ANXA1 and/or PPP3CB.

A high/low level of binding is preferably relative to a level of binding between the same binding means and polypeptide under the same conditions for a granulocyte that is unsuitable for treating cancer.

The term “isolating” may mean providing a population of granulocytes in which at least 50%, 60%, 70%, 80% or 90% (preferably at least 95%, 99% or 100%) are granulocytes suitable for treating cancer. In other words, the term “isolating” may mean removing at least 50%, 60%, 70%, 80% or 90% (preferably at least 95%, 99% or 100%) of granulocytes that are unsuitable for treating cancer from a population of granulocytes.

Thus, the methods for isolating suitably allow for the separation of a granulocyte for treating cancer from granulocyte that is unsuitable for treating cancer.

In some embodiments a method described herein comprises discarding granulocytes that are unsuitable for treating cancer.

In one aspect the invention provides a pharmaceutical composition comprising:
a. a granulocyte, stem cell, or composition of the invention; and
b. a pharmaceutically acceptable carrier, excipient, adjuvant, and/or salt

The term “pharmaceutically acceptable carrier, excipient, adjuvant, and/or salt” as used herein means a carrier that can be administered to a subject (e.g. a patient) intravenously, intra-arterially, intraperitoneally, intra-tumourally, intrathecally or combinations thereof (preferably intravenously) without causing harm to said subject. In one embodiment a pharmaceutically acceptable carrier is an injectable carrier, such as a sterile physiological saline solution. In one embodiment a pharmaceutically acceptable carrier, excipient, adjuvant, and/or salt may be Plasma-Lyte A (e.g. commercially available from Baxter, USA), dextrose, sodium chloride, human serum albumin, dextran (e.g. dextran 40 (LMD)), dextrose, DMS or combinations thereof. Plasma-Lyte A may be present at a concentration of 10-50% v/v (preferably 31.25% v/v). 5% dextrose/0.45% sodium chloride may be present at a concentration of 10-50% v/v (preferably 31.25% v/v). 25% HAS may be present at 10-30% v/v (preferably 20% v/v). 10% Dextran 40 (LMD)/5% dextrose may be present at a concentration of 1-30% v/v (preferably 10% v/v). DMS may be present at 1-15% v/v (preferably 7.5% v/v).

The pharmaceutical composition may comprise a granulocyte-macrophage colony-stimulating factor (GM-CSF), a granulocyte colony-stimulating factor (G-CSF), a growth hormone; serotonin, vitamin C, vitamin D, glutamine (Gln), arachidonic acid, AGE-albumin, an interleukin, TNF-alpha, Flt-3 ligand, thrombopoietin, serum (e.g. foetal bovine serum [FBS]), retinoic acid, lipopolysaccharide (LPS), IFN-gamma, IFN-beta, or combinations thereof. Suitably, the pharmaceutical composition comprises IFN-gamma and a GM-CSF. Preferably, the pharmaceutical composition comprises TNF-alpha. Particularly preferably, the pharmaceutical composition comprises a granulocyte-macrophage colony-stimulating factor (GM-CSF), and a granulocyte colony-stimulating factor (G-CSF), and a growth hormone, and serotonin, and vitamin C, and vitamin D, and glutamine (Gln), and arachidonic acid, and AGE-albumin, and an interleukin, and TNF-alpha, and Flt-3 ligand, and thrombopoietin, and foetal bovine serum (FBS). Preferably, the pharmaceutical composition comprises a granulocyte-macrophage colony-stimulating factor (GM-CSF), and a granulocyte colony-stimulating factor (G-CSF), and a growth hormone, and serotonin, and vitamin C, and vitamin D, and glutamine (Gln), and arachidonic acid, and AGE-albumin, and an interleukin, and TNF-alpha, and Flt-3 ligand, and thrombopoietin, and foetal bovine serum (FBS), and retinoic acid, and lipopolysaccharide (LPS), and IFN-gamma, and IFN-beta.

In a related aspect the invention provides a kit comprising a granulocyte, stem cell, composition or pharmaceutical composition of the invention; and instructions for use of the same in medicine (e.g. in treating cancer). Suitably, the instructions may be for the use of the same in treating a cancer described in any one of the foregoing embodiments. In some embodiments the instructions also detail an appropriate dosage regimen (e.g. as described in a foregoing embodiment). In one embodiment the instructions are for use of said kit in treating cancer, preferably pancreatic cancer.

The invention may further comprise depositing a granulocyte, stem cell, composition or pharmaceutical composition of the invention in a cell bank, and thus in a related aspect provides a granulocyte, stem cell, composition or pharmaceutical composition. The term “cell bank” as used herein refers to a storage facility which maintains a cell under suitable conditions for cell viability. For example, the cell may be stored in a metabolically dormant state (e.g. cryogenically frozen). Suitably, a cell comprised within a cell bank is catalogued for appropriate retrieval (e.g. based on blood group, and/or human leukocyte antigen (HLA) type). In one embodiment a cell may be catalogued based on the type of cancer it (or a cell differentiated therefrom) kills. Where the cell bank is a granulocyte cell bank, said cell bank may be replenished using a stem cell of the invention. In some embodiments a stem cell or granulocyte obtained from a donor may be stored and later administered to said donor (e.g. if said donor is diagnosed with cancer), thus constituting a personalised medicine.

A granulocyte or stem cell of the invention may be formulated in any suitable manner, based on its downstream application (e.g. storage in a cell bank, or use in therapy).

Thus, one aspect of the invention provides a cell bank comprising the stem cell, granulocyte, composition, or pharmaceutical composition of the present invention.

The present invention provides granulocytes, stem cells, pharmaceutical compositions, and kits for use in medicine, particularly in the treatment of cancer.

Thus in one aspect the invention provides a granulocyte of the invention for use in treating cancer. In another aspect the invention provides a stem cell of the invention for use in treating cancer. In another aspect the invention provides a composition of the invention for use in treating cancer. In another aspect the invention provides a pharmaceutical composition of the invention for use in treating cancer. In another aspect the invention provides a kit of the invention for use in treating cancer. Similarly, the invention provides in one aspect use of a granulocyte, stem cell, composition, pharmaceutical composition, or kit of the invention in the manufacture of a medicament for treating cancer. In a related aspect there is provided a method for treating cancer comprising: administering to a subject in need thereof a granulocyte, stem cell, composition, pharmaceutical composition, or kit of the invention.

In one embodiment a cancer is a solid tumour cancer. The term “solid tumour cancer” refers to an abnormal, malignant mass of tissue that does not contain cysts or liquid inclusions. Examples of solid tumour cancers include carcinomas, sarcomas, and lymphomas.

A solid tumour cancer may be a carcinoma. A carcinoma may be selected from one or more of an adenocarcinoma, a basal cell carcinoma, a squamous cell carcinoma, an adenosquamous carcinoma, a renal cell carcinoma, a ductal carcinoma in situ (DCIS), an invasive ductal carcinoma, an anaplastic carcinoma, a large cell carcinoma, a small cell carcinoma or combinations thereof. A carcinoma may also be selected from epithelial neoplasms, squamous cell neoplasms, squamous cell carcinoma, basal cell neoplasms, basal cell carcinoma, transitional cell carcinomas, adenocarcinomas (such as Adenocarcinoma not otherwise specified (NOS), linitis plastica, vipoma, cholangiocarcinoma, hepatocellular carcinoma NOS, adenoid cystic carcinoma, renal cell carcinoma, Grawitz tumour), adnexal and skin appendage neoplasms, mucoepidermoid neoplasms, cystic mucinous and serous neoplasms, ductal lobular and medullary neoplasms, acinar cell neoplasms, or complex epithelial neoplasms.

Alternatively a solid tumour cancer may be a sarcoma. A sarcoma may be selected from Askin’s tumour, sarcoma botryoides, chondrosarcoma, Ewing’s, malignant hemangioendothelioma, malignant schwannoma, osteosarcoma, or soft tissue sarcomas (including alveolar soft part sarcoma, angiosarcoma, cystosarcoma phyllodes, dermatofibrosarcoma protuberans (DFSP), desmoid tumour, desmoplastic small round cell tumour, epithelioid sarcoma, extraskeletal chondrosarcoma, extraskeletal osteosarcoma, fibrosarcoma, gastrointestinal stromal tumour (GIST), hemangiopericytoma, hemangiosarcoma, Kaposi’s sarcoma, leiomyosarcoma, liposarcoma, lymphangiosarcoma, malignant fibrous histiocytoma, undifferentiated pleomorphic sarcoma, malignant peripheral nerve sheath tumour (MPNST), neurofibrosarcoma, rhabdomyosarcoma, and synovial sarcoma).

Alternatively a solid tumour may be a lymphoma, such as a B-cell lymphoma, a T-cell lymphoma, a NK-cell lymphoma, or a Hodgkin’s lymphoma.

In one embodiment a granulocyte, stem cell, composition, pharmaceutical composition or kit of the invention is for use in treating one or more of: pancreatic cancer, liver cancer, oesophageal cancer, stomach cancer, cervical cancer, ovarian cancer, lung cancer, bladder cancer, kidney cancer, brain cancer, prostate cancer, myeloma cancer, non-Hodgkin’s lymphoma (NHL), larynx cancer, uterine cancer, or breast cancer.

Preferably a granulocyte, stem cell, composition, pharmaceutical composition or kit of the invention is for use in treating pancreatic cancer. The pancreatic cancer may be a pancreatic solid tumour cancer, such as a pancreatic adenocarcinoma (e.g. a pancreatic ductal adenocarcinoma).

Pancreatic cancer is known to be one of the most difficult cancers to treat. However surprisingly, the present inventors have succeeded in providing granulocytes (and stem cells that differentiate into granulocytes) that have particular efficacy against pancreatic cancer cells.

In some embodiments a stem cell may be differentiated into a granulocyte prior to administration. In preferred embodiments a stem cell may be differentiated into a different stem cell (preferably a precursor cell) prior to administration. In other embodiments a stem cell may be administered to a subject. A stem cell may be administered by any suitable technique known in the art. In one embodiment a subject may be given a stem cell transplant, such as a bone marrow transplant.

Prior to administration there may be a matching step between a medicament of the invention (e.g. a granulocyte, stem cell, composition, pharmaceutical composition or kit of the invention) and the subject to be treated. Matching may be based on data derived from the donor from which the stem cell, or granulocyte is derived, and similar data obtained from the subject to be treated. Matching may be achieved on the basis of blood group type, human leukocyte antigen (HLA) type similarity, or combinations thereof.

A granulocyte, stem cell, composition, pharmaceutical composition or kit of the invention may be administered to a subject in a therapeutically effective amount or a prophylactically effective amount.

The term “treat” or “treating” as used herein encompasses prophylactic treatment (e.g. to prevent onset of a disease) as well as corrective treatment (treatment of a subject already suffering from a disease). Preferably “treat” or “treating” as used herein means corrective treatment.

The term “treat” or “treating” as used herein refers to the disorder and/or a symptom thereof.

A “therapeutically effective amount” is any amount of the granulocyte, stem cell, composition, pharmaceutical composition or kit of the invention, which when administered alone or in combination to a subject for treating cancer (or a symptom thereof) is sufficient to effect such treatment of the disorder, or symptom thereof.

A “prophylactically effective amount” is any amount of the granulocyte, stem cell, composition, pharmaceutical composition or kit of the invention that, when administered alone or in combination to a subject inhibits or delays the onset or reoccurrence of cancer (or a symptom thereof). In some embodiments, the prophylactically effective amount prevents the onset or reoccurrence of a cancer entirely. “Inhibiting” the onset means either lessening the likelihood of cancer onset (or symptom thereof), or preventing the onset entirely.

In one embodiment a granulocyte is administered to a subject. Preferably, the granulocyte is a neutrophil.

In one embodiment a stem cell is administered to a subject. Preferably, the stem cell is a precursor cell, e.g. selected from a common myeloid progenitor cell, a myeloblast, a promyelocyte (e.g. a N. promyelocyte), a myelocyte (e.g. a N. myelocyte), a metamyelocyte (e.g. a N. metamyelocyte), a band (e.g. an N. band), or combinations thereof.

In some embodiments a stem cell and a granulocyte are administered to a subject. Preferably, a precursor cell and a neutrophil are administered to a subject.

An appropriate dosage range is one that produces the desired therapeutic effect (e.g. wherein the granulocyte, stem cell, composition, pharmaceutical composition or kit of the invention is dosed in a therapeutically or prophylactically effective amount).

A typical treatment regimen may include administering from 10⁶, 10⁷, 10⁸ or 10⁹ cells (e.g. granulocyte cells or stem cells) to a subject, or up to 10¹², 10¹³ or 10¹⁴ cells to a subject. In one embodiment a treatment regimen includes administering a dose of at least 1 × 10⁹ cells to a subject. Suitably, a treatment regimen may include administering a dose of at least 2 × 10⁹ cells or at least 5 × 10⁹ cells to a subject. In one embodiment a treatment regimen may include administering a dose of at least 1 × 10¹⁰ cells or at least 5 × 10¹⁰ cells to a subject. At least 1 × 10¹¹ or at least 2 × 10¹¹ cells may be administered to a subject. In some embodiments between 1 × 10⁹ to 3 × 10¹¹ or 1 × 10¹⁰ to 3 × 10¹¹ cells are administered to a subject. Suitably, between 5 × 10¹⁰ to 2.5 × 10¹¹ cells are administered to a subject. In one embodiment when the cell is a stem cell, e.g. a precursor cell as defined herein, a treatment regimen includes administering a dose between 1/100^{th} and 1/700^{th}, preferably a dose between 1/200^{th} and 1/400^{th}, such as 1/300^{th}, of the dose when compared to the dose of granulocytes administered.

A subject for treatment may be dosed once, twice, three times, four times, five times, or six times per week. Alternatively a subject may be dosed daily (e.g. once or twice daily). In other embodiments a subject may be dosed once weekly or bi-weekly. Preferably the dose is weekly. The skilled person will appreciate that the dose can be tailored based on the needs of the subject, and efficacy of the medicament. For example, where the medicament is highly efficacious, the dose may be lowered.

In one embodiment a subject for treatment is dosed weekly (e.g. once weekly) with at least 2 × 10⁹ cells or at least 2 × 10¹⁰ cells. Suitably, a subject for treatment may be dosed weekly with at least 1 × 10¹¹ or at least 2 × 10¹¹ cells.

The treatment term can be varied based on the response of the subject to the treatment, and/or the type and/or severity of the cancer. For example, the subject for treatment may be dosed for at least 1 or 2 weeks. Suitably the subject for treatment may be dosed for at least 3 or 4 weeks. In one embodiment the subject for treatment is dosed for at least 5 or 6 weeks, suitably at least 7 or 8 weeks.

In one embodiment a subject for treatment is dosed for 4-8 weeks with at least 2 × 10⁹ cells, wherein said cells are administered once weekly. Suitably a subject for treatment is dosed for 8 weeks with at least 2 × 10⁹ cells (preferably at least 2 × 10¹⁰ or 2 × 10¹¹ cells), wherein said cells are administered once weekly.

Administration may be by any suitable technique or route, including but not limited to intravenous injection, intra-arterial injection, intraperitoneal injection, injection into a tumour resection cavity, intrathecal injection, or combinations thereof. Suitably the medicament may be administered intravenously.

A white blood cell growth factor may be administered with a medicament of the invention. The administration may be sequential or simultaneous (suitably simultaneous). Suitable white blood cell growth factors may include a granulocyte-macrophage colony-stimulating factor (GM-CSF), a granulocyte colony-stimulating factor (G-CSF), a growth hormone; serotonin, vitamin C, vitamin D, glutamine (Gln), arachidonic acid, AGE-albumin, an interleukin, TNF-alpha, Flt-3 ligand, thrombopoietin, foetal bovine serum (FBS), retinoic acid, lipopolysaccharide (LPS), IFN-gamma, IFN-beta, or combinations thereof. Suitably, the white blood cell growth factors comprises IFN-gamma and GM-CSF. Preferably, the white blood cell growth factors comprises TNF-alpha. Suitably the white blood cell growth factors may comprise a granulocyte-macrophage colony-stimulating factor (GM-CSF), and a granulocyte colony-stimulating factor (G-CSF), and a growth hormone, and serotonin, and vitamin C, and vitamin D, and glutamine (Gln), and arachidonic acid, and AGE-albumin, and an interleukin, and TNF-alpha, and Flt-3 ligand, and thrombopoietin, and foetal bovine serum (FBS). Suitably the white blood cell growth factors may comprise a granulocyte-macrophage colony-stimulating factor (GM-CSF), and a granulocyte colony-stimulating factor (G-CSF), and a growth hormone, and serotonin, and vitamin C, and vitamin D, and glutamine (Gln), and arachidonic acid, and AGE-albumin, and an interleukin, and TNF-alpha, and Flt-3 ligand, and thrombopoietin, and foetal bovine serum (FBS), and retinoic acid, and lipopolysaccharide (LPS), and IFN-gamma, and IFN-beta. Particular examples of the foregoing include but are not limited to LEUKINE® brand sargramostim, NEUPOGEN® brand filgrastim, and NEULAST A® brand 5 PEG-filgrastim.

In one embodiment a stem cell may be administered (e.g. sequentially or simultaneously, preferably simultaneously) with a granulocyte-colony stimulating factor; and a growth hormone; and a serotonin; and an interleukin. In one embodiment a granulocyte precursor cell (e.g. a granulocyte precursor cell culture) is administered (e.g. sequentially or simultaneously, preferably simultaneously) with a granulocyte-colony stimulating factor; and a growth hormone; and a serotonin; and an interleukin.

In some embodiments a granulocyte or stem cell of the invention may be used in combination with another therapeutic, e.g. in combination with an existing cancer therapy, such as radiotherapy, chemotherapy, and/or immunotherapy.

In one aspect the present invention provides a method for determining the suitability of a stem cell for treating cancer, the method comprising:
a. comparing a measured expression level of one or more genes by the stem cell, wherein the one or more genes are associated with suitability for treating cancer and are selected from: CTSG, CAP37, ITGB1, CYBB, SYK, DOCK8, COMP, ATG7, SLC2A1, GZMK, ATM, IKBKB, BCAP31, TAPBP, PPP3CB, ANXA1, PERM, PLEC, ACSL1, RAC1, GM2A, and PSMB2, with the expression level of the same one or more genes in a reference standard; and
b. determining the suitability of the stem cell for treating cancer based on the comparison.

In one aspect the present invention provides a method for determining the suitability of a stem cell for treating cancer, the method comprising:
a. measuring an expression level of one or more genes by the stem cell, wherein the one or more genes are associated with suitability for treating cancer and are selected from: ITGB1, CYBB, SYK, DOCK8, COMP, ATG7, SLC2A1, GZMK, CTSG, ATM, IKBKB, BCAP31, TAPBP, PPP3CB, ANXA1, PERM, PLEC, ACSL1, RAC1, GM2A, CAP37, and PSMB2;
b. comparing the measured expression level with the expression level of the same one or more genes in a reference standard; and
c. determining the suitability of the stem cell for treating cancer based on the comparison.

In another aspect the invention provides a method for identifying whether or not a donor produces stem cells suitable for treating cancer, the method comprising:
a. comparing a measured expression level of one or more genes by a stem cell comprised in a sample obtainable from the donor, wherein the one or more genes are associated with suitability for treating cancer and are selected from: CTSG, CAP37, ITGB1, CYBB, SYK, DOCK8, COMP, ATG7, SLC2A1, GZMK, ATM, IKBKB, BCAP31, TAPBP, PPP3CB, ANXA1, PERM, PLEC, ACSL1, RAC1, GM2A, and PSMB2, with the expression level of the same one or more genes in a reference standard; and
b. identifying whether or not the donor produces stem cells suitable for treating cancer based on the comparison.

In a related aspect the invention provides a method for identifying whether or not a donor produces stem cells for treating cancer, the method comprising:
a. measuring an expression level of one or more genes by a stem cell comprised in a sample obtainable from the donor, wherein the one or more genes are associated with suitability for treating cancer and are selected from: ITGB1, CYBB, SYK, DOCK8, COMP, ATG7, SLC2A1, GZMK, CTSG, ATM, IKBKB, BCAP31, TAPBP, PPP3CB, ANXA1, PERM, PLEC, ACSL1, RAC1, GM2A, CAP37, and PSMB2;
b. comparing the measured expression level with the expression level of the same one or more genes in a reference standard; and
c. identifying whether or not the donor produces stem cells for treating cancer based on the comparison.

In one embodiment a stem cell is determined to be suitable for treating cancer or a donor is identified as producing stem cells suitable for treating cancer when:
i. a measured expression level of one or more of ITGB1, CYBB, SYK, DOCK8, COMP, ATG7, SLC2A1, GZMK, CTSG, ATM, IKBKB, BCAP31, TAPBP, PERM, PLEC, ACSL1, RAC1, GM2A, CAP37, and PSMB2 is increased when compared to the reference standard when the reference standard is from a stem cell unsuitable for treating cancer; or
ii. a measured expression level of ANXA1 and/or PPP3CB is decreased when compared to the reference standard, when the reference standard is from a stem cell unsuitable for treating cancer; or
iii. a measured expression level of one or more of ITGB1, CYBB, SYK, DOCK8, COMP, ATG7, SLC2A1, GZMK, CTSG, ATM, IKBKB, BCAP31, TAPBP, PERM, PLEC, ACSL1, RAC1, GM2A, CAP37, and PSMB2 is increased or the same when compared to the reference standard, when the reference standard is from a stem cell suitable for treating cancer; or
iv. a measured expression level of ANXA1 and/or PPP3CB is decreased or the same when compared to the reference standard, when the reference standard is from a stem cell suitable for treating cancer.

Alternatively, in one embodiment a stem cell is determined to be unsuitable for treating cancer or a donor is identified as producing stem cells unsuitable for treating cancer when:
i. a measured expression level of one or more of ITGB1, CYBB, SYK, DOCK8, COMP, ATG7, SLC2A1, GZMK, CTSG, ATM, IKBKB, BCAP31, TAPBP, PERM, PLEC, ACSL1, RAC1, GM2A, CAP37, and PSMB2 is decreased or the same when compared to the reference standard, when the reference standard is from a stem cell unsuitable for treating cancer; or
ii. a measured expression level of ANXA1 and/or PPP3CB is increased or the same when compared to the reference standard, when the reference standard is from a stem cell unsuitable for treating cancer; or
iii. a measured expression level of one or more of ITGB1, CYBB, SYK, DOCK8, COMP, ATG7, SLC2A1, GZMK, CTSG, ATM, IKBKB, BCAP31, TAPBP, PERM, PLEC, ACSL1, RAC1, GM2A, CAP37, and PSMB2 is decreased when compared to reference standard, when the reference standard is from a stem cell suitable for treating cancer; or
iv. a measured expression level of ANXA1 and/or PPP3CB is increased when compared to the reference standard, when the reference standard is from a stem cell suitable for treating cancer.

In one aspect the invention provides a stem cell, wherein the stem cell comprises:
a. increased expression of one or more of ITGB1, CYBB, SYK, DOCK8, COMP, ATG7, SLC2A1, GZMK, CTSG, ATM, IKBKB, BCAP31, TAPBP, PERM, PLEC, ACSL1, RAC1, GM2A, CAP37, and PSMB2 when compared to a reference standard, wherein the reference standard is from a stem cell unsuitable for treating cancer; and/or
b. decreased expression of ANXA1 and/or PPP3CB when compared to a reference standard, wherein the reference standard is from a stem cell unsuitable for treating cancer.

In one aspect the invention provides a method for selecting whether or not a subject is suitable for treatment with a stem cell or granulocyte for treating cancer, the method comprising:
a. comparing a measured expression level of one or more genes by a stem cell comprised in a sample obtainable from the subject, wherein the one or more genes are associated with suitability for treating cancer and are selected from: CTSG, CAP37, ITGB1, CYBB, SYK, DOCK8, COMP, ATG7, SLC2A1, GZMK, ATM, IKBKB, BCAP31, TAPBP, PPP3CB, ANXA1, PERM, PLEC, ACSL1, RAC1, GM2A, and PSMB2 with the expression level of the same one or more genes in a reference standard; and
b. identifying whether or not the subject is suitable for treatment with a stem cell or granulocyte for treating cancer based on the comparison.

In one aspect the invention provides a method for selecting whether or not a subject is suitable for treatment with a stem cell or granulocyte for treating cancer, the method comprising:
a. measuring an expression level of one or more genes by a stem cell comprised in a sample obtainable from the subject, wherein the one or more genes are associated with suitability for treating cancer and are selected from: CTSG, CAP37, ITGB1, CYBB, SYK, DOCK8, COMP, ATG7, SLC2A1, GZMK, ATM, IKBKB, BCAP31, TAPBP, PPP3CB, ANXA1, PERM, PLEC, ACSL1, RAC1, GM2A, and PSMB2;
b. comparing the measured expression level with the expression level of the same one or more genes in a reference standard; and
c. identifying whether or not the subject is suitable for treatment with a stem cell or granulocyte for treating cancer based on the comparison.

In another aspect the invention provides a method for determining a subject’s risk for developing cancer, the method comprising:
a. comparing a measured expression level of one or more genes by a stem cell comprised in a sample obtainable from the subject, wherein the one or more genes are associated with suitability for treating cancer and are selected from: CTSG, CAP37, ITGB1, CYBB, SYK, DOCK8, COMP, ATG7, SLC2A1, GZMK, ATM, IKBKB, BCAP31, TAPBP, PPP3CB, ANXA1, PERM, PLEC, ACSL1, RAC1, GM2A, and PSMB2 with the expression level of the same one or more genes in a reference standard; and
b. determining the subject’s risk for developing cancer based on the comparison.

In another aspect the invention provides a method for determining a subject’s risk for developing cancer, the method comprising:
a. measuring an expression level of one or more genes by a stem cell comprised in a sample obtainable from the subject, wherein the one or more genes are associated with suitability for treating cancer and are selected from: CTSG, CAP37, ITGB1, CYBB, SYK, DOCK8, COMP, ATG7, SLC2A1, GZMK, ATM, IKBKB, BCAP31, TAPBP, PPP3CB, ANXA1, PERM, PLEC, ACSL1, RAC1, GM2A, and PSMB2;
b. comparing the measured expression level with the expression level of the same one or more genes in a reference standard; and
c. determining the subject’s risk for developing cancer based on the comparison.

In one embodiment, step c. of any one of the foregoing aspects comprises: identifying the subject as suitable for treatment with a granulocyte or a stem cell for treating cancer or determining that the subject is at risk of developing cancer when:
i. the measured expression level of one or more of CTSG, CAP37, ITGB1, CYBB, SYK, DOCK8, COMP, ATG7, SLC2A1, GZMKATM, IKBKB, BCAP31, TAPBP, PERM, PLEC, ACSL1, RAC1, GM2A, and PSMB2 is decreased or the same when compared to the reference standard, when the reference standard is from a stem cell unsuitable for treating cancer; or
ii. the measured expression level of ANXA1 and/or PPP3CB is increased or the same when compared to the reference standard, when the reference standard is from a stem cell unsuitable for treating cancer; or
iii. the measured expression level of one or more of CTSG, CAP37, ITGB1, CYBB, SYK, DOCK8, COMP, ATG7, SLC2A1, GZMK, ATM, IKBKB, BCAP31, TAPBP, PERM, PLEC, ACSL1, RAC1, GM2A, and PSMB2 is decreased when compared to the reference standard, when the reference standard is from a stem cell suitable for treating cancer; or
iv. the measured expression level of ANXA1 and/or PPP3CB is increased when compared to the reference standard, when the reference standard is from a stem cell suitable for treating cancer; or identifying the subject as unsuitable for treatment with a granulocyte or a stem cell for treating cancer when or determining that the subject is not at risk of developing cancer when:
v. the measured expression level of one or more of CTSG, CAP37, ITGB1, CYBB, SYK, DOCK8, COMP, ATG7, SLC2A1, GZMK, ATM, IKBKB, BCAP31, TAPBP, PERM, PLEC, ACSL1, RAC1, GM2A, and PSMB2 is increased when compared to the reference standard when the reference standard is from a stem cell unsuitable for treating cancer; or
vi. the measured expression level of ANXA1 and/or PPP3CB is decreased when compared to the reference standard, when the reference standard is from a stem cell unsuitable for treating cancer; or
vii. the measured expression level of one or more of CTSG, CAP37, ITGB1, CYBB, SYK, DOCK8, COMP, ATG7, SLC2A1, GZMK, ATM, IKBKB, BCAP31, TAPBP, PERM, PLEC, ACSL1, RAC1, GM2A, and PSMB2 is increased or the same when compared to the reference standard, when the reference standard is from a stem cell suitable for treating cancer; or
viii. the measured expression level of ANXA1 and/or PPP3CB is decreased or the same when compared to the reference standard, when the reference standard is from a stem cell suitable for treating cancer.

In some embodiments of any of the foregoing aspects, the method does not comprise measuring the expression level of CD10 and/or CD101 by a granulocyte or stem cell comprised in a sample from a donor. In some embodiments of any of the foregoing aspects, the method does not comprise comparing the measured expression level of CD10 and/or CD101 with the expression level with the same genes in a reference standard.

In alternative embodiments, the method comprises measuring the expression level of CD10 and/or CD101 by a granulocyte or stem cell comprised in a sample from a donor. In some embodiments of any of the foregoing aspects, the method comprises comparing the measured expression level of CD10 and/or CD101 with the expression level with the same genes in a reference standard.

In one aspect the invention provides a composition for treating cancer, the composition comprising stem cells: wherein at least 90% (preferably at least 95%, 99% or 100%) of the stem cells comprised in the composition have:
a. increased expression of one or more of CTSG, CAP37, ITGB1, CYBB, SYK, DOCK8, COMP, ATG7, SLC2A1, GZMK, ATM, IKBKB, BCAP31, TAPBP, PERM, PLEC, ACSL1, RAC1, GM2A, and PSMB2ITGB1, CYBB, SYK, DOCK8, COMP, ATG7, SLC2A1, GZMK, CTSG, ATM, IKBKB, BCAP31, TAPBP, PERM, PLEC, ACSL1, RAC1, GM2A, CAP37, and PSMB2 when compared to a reference standard, wherein the reference standard is from a stem cell unsuitable for treating cancer; and/or
b. decreased expression of ANXA1 and/or PPP3CB when compared to a reference standard, wherein the reference standard is from a stem cell unsuitable for treating cancer.

The invention also provides a method for isolating stem cells suitable for treating cancer based on the expression of one or more genes of the invention. Such methods may provide a substantially homogeneous population of stem cells that are suitable for treating cancer. In one embodiment, stem cells for treating cancer are isolated using the expression of one or more cell-surface expressed polypeptides selected from: ATG7, CYBB, DOCK8, CTSG, S100A9, COMP, S100A8, CTSG, SYK, ITGB1, SLC2A1, GZMK, ANXA1, RAC1, and CAP37, preferably one or more selected from: ATG7, S100A9, COMP, S100A8, CTSG, SYK, ITGB1, SLC2A1, GZMK, ANXA1, RAC1, and CAP37. The isolation may be performed using any suitable technique. In one embodiment a method for isolating granulocytes comprises the use of a binding means that binds to a polypeptide of the invention. Preferably the binding means is an antibody. Antibodies to detect the presence or absence of polypeptides of the invention are commercially available and may be one or more of the antibodies described herein. The method may comprise the use of flow cytometric techniques, preferably fluorescence activated cell sorting (FACS), e.g. together with appropriate ‘gating’. Flow cytometric techniques may be particularly suitable when the method employs the use of a binding means coupled to a detectable label, such as a fluorophore.

In one aspect there is provided a method for isolating a stem cell for treating cancer, the method comprising:
a) contacting a sample of stem cells with a binding means, wherein the binding means binds to one or more polypeptides selected from CTSG, CAP37, ITGB1, CYBB, SYK, DOCK8, COMP, ATG7, SLC2A1, GZMK, ATM, IKBKB, BCAP31, TAPBP, PPP3CB, ANXA1, PERM, PLEC, ACSL1, RAC1, GM2A, and PSMB2; and
b) isolating the stem cell based on the presence or absence of binding between the binding means and the one or more polypeptides.

Binding may be determined to be present when the amount of binding is statistically significant (e.g. when compared to a ‘background’ control). Binding may be determined to be absent when the amount of binding is statistically insignificant (e.g. when compared to a ‘background’ control). Preferably, binding is determined to be absent when there is no binding whatsoever.

In one embodiment the invention comprises detecting the presence of one or more polypeptides selected from CTSG, CAP37, ITGB1, CYBB, SYK, DOCK8, COMP, ATG7, SLC2A1, GZMK, ATM, IKBKB, BCAP31, TAPBP, PERM, PLEC, ACSL1, RAC1, GM2A, and PSMB2. When said one or more polypeptides are detected (i.e. where there is binding between the binding means and the polypeptide) the stem cell may be isolated. Suitably, said isolated stem cell may be a stem cell for treating cancer.

In another embodiment the invention may comprise detecting the absence of ANXA1 and/or PPP3CB (e.g. not detecting ANXA1 and/or PPP3CB). When said one or more polypeptides are not detected (i.e. where there is an absence of binding between the binding means and the polypeptide) the stem cell may be isolated. Suitably, said isolated stem cell may be a stem cell for treating cancer.

Preferably, the invention comprises detecting:
the presence of one or more polypeptides selected from CTSG, CAP37, ITGB1, CYBB, SYK, DOCK8, COMP, ATG7, SLC2A1, GZMK, ATM, IKBKB, BCAP31, TAPBP, PERM, PLEC, ACSL1, RAC1, GM2A, and PSMB2; and
the invention absence of ANXA1 and/or PPP3CB. In one embodiment, when said polypeptides are detected or not detected (as indicated), the stem cell is isolated.

In one embodiment a method of isolating a stem cell comprises the use of an immobilised binding means (e.g. a binding means conjugated to a bead, such as a magnetic bead, or chromatographic resin) to isolate a stem cell of the invention. Such methods may be immuno-affinity methods.

The method may comprise quantifying the amount of binding between the binding means and the one or more polypeptides or between the binding means and the stem cell. The method may comprise isolating a stem cell for treating cancer based on the quantified amount of binding.

In one embodiment a stem cell for treating cancer is isolated when there is a high level of binding between a binding means and one or more polypeptides selected from CTSG, CAP37, ITGB1, CYBB, SYK, DOCK8, COMP, ATG7, SLC2A1, GZMK, ATM, IKBKB, BCAP31, TAPBP, PERM, PLEC, ACSL1, RAC1, GM2A, and PSMB2.

In one embodiment a stem cell for treating cancer is isolated when there is a low level of binding between a binding means and ANXA1 and/or PPP3CB.

Preferably, a stem cell for treating cancer is isolated when there is:
a high level of binding between a binding means and one or more polypeptides selected from CTSG, CAP37, ITGB1, CYBB, SYK, DOCK8, COMP, ATG7, SLC2A1, GZMK, ATM, IKBKB, BCAP31, TAPBP, PERM, PLEC, ACSL1, RAC1, GM2A, and PSMB2; and
a low level of binding between a binding means and ANXA1 and/or PPP3CB.

A high/low level of binding is preferably relative to a level of binding between the same binding means and polypeptide under the same conditions for a stem cell that is unsuitable for treating cancer.

The term “isolating” may mean providing a population of stem cells in which at least 50%, 60%, 70%, 80% or 90% (preferably at least 95%, 99% or 100%) are stem cells suitable for treating cancer. In other words, the term “isolating” may mean removing at least 50%, 60%, 70%, 80% or 90% (preferably at least 95%, 99% or 100%) of stem cells that are unsuitable for treating cancer from a population of stem cells.

Thus, the methods for isolating suitably allow for the separation of a stem cell for treating cancer from stem cell that is unsuitable for treating cancer.

In some embodiments a method described herein comprises discarding stem cells that are unsuitable for treating cancer.

In one aspect the invention provides a stem cell for treating cancer obtainable by a method of the invention (e.g. a stem cell capable of differentiating into granulocytes that are suitable to treat cancer).

In one aspect there is provided a method for producing a stem cell for treating cancer, the method comprising:
a. providing a cell; and
b. converting the cell into a stem cell having an expression profile described herein, for example a stem cell that is capable of differentiating into a granulocyte having an expression profile described herein, e.g. wherein:
   i. the measured expression level of one or more of GM2A, CTSG, CAP37, ITGB1, CYBB, SYK, DOCK8, COMP, ATG7, SLC2A1, GZMK, ATM, IKBKB, BCAP31, TAPBP, PERM, PLEC, ACSL1, RAC1, and PSMB2 is increased in the granulocyte when compared to a reference standard when the reference standard is from a granulocyte unsuitable for treating cancer; or
   ii. the measured expression level of ANXA1 and/or PPP3CB is decreased in the granulocyte when compared to the reference standard, when the reference standard is from a granulocyte unsuitable for treating cancer; or
   iii. the measured expression level of one or more of GM2A, CTSG, CAP37, ITGB1, CYBB, SYK, DOCK8, COMP, ATG7, SLC2A1, GZMK, ATM, IKBKB, BCAP31, TAPBP, PERM, PLEC, ACSL1, RAC1, and PSMB2 is increased or the same when compared to the reference standard, when the reference standard is from a granulocyte suitable for treating cancer; or
   iv. the measured expression level of ANXA1 and/or PPP3CB is decreased or the same when compared to the reference standard, when the reference standard is from a granulocyte suitable for treating cancer; and
c. optionally isolating the stem cell.

In one embodiment the cell is a somatic/differentiated cell, optionally from a donor who produces granulocytes suitable for treating cancer, for example as determined according to a method of the invention.

In some embodiments of any of the foregoing aspects, a granulocyte unsuitable for treating cancer is a viable granulocyte.

Embodiments related to the various methods of the invention are intended to be applied equally to other methods, the granulocytes, stem cells, compositions, pharmaceutical compositions or uses, and vice versa.

### SEQUENCE IDENTITY

Any of a variety of sequence alignment methods can be used to determine percent identity, including, without limitation, global methods, local methods and hybrid methods, such as, e.g., segment approach methods. Protocols to determine percent identity are routine procedures within the scope of one skilled in the art. Global methods align sequences from the beginning to the end of the molecule and determine the best alignment by adding up scores of individual residue pairs and by imposing gap penalties. Non-limiting methods include, e.g., CLUSTAL W, see, e.g., Julie D. Thompson et al., CLUSTAL W: Improving the Sensitivity of Progressive Multiple Sequence Alignment Through Sequence Weighting, Position- Specific Gap Penalties and Weight Matrix Choice, 22(22) Nucleic Acids Research 4673-4680 (1994); and iterative refinement, see, e.g., Osamu Gotoh, Significant Improvement in Accuracy of Multiple Protein. Sequence Alignments by Iterative Refinement as Assessed by Reference to Structural Alignments, 264(4) J. Mol. Biol. 823-838 (1996). Local methods align sequences by identifying one or more conserved motifs shared by all of the input sequences. Non-limiting methods include, e.g., Match-box, see, e.g., Eric Depiereux and Ernest Feytmans, Match-Box: A Fundamentally New Algorithm for the Simultaneous Alignment of Several Protein Sequences, 8(5) CABIOS 501 -509 (1992); Gibbs sampling, see, e.g., C. E. Lawrence et al., Detecting Subtle Sequence Signals: A Gibbs Sampling Strategy for Multiple Alignment, 262(5131) Science 208-214 (1993); Align-M, see, e.g., Ivo Van Walle et al., Align-M - A New Algorithm for Multiple Alignment of Highly Divergent Sequences, 20(9) Bioinformatics: 1428-1435 (2004).

Thus, percent sequence identity is determined by conventional methods. See, for example, Altschul et al., Bull. Math. Bio. 48: 603-16, 1986 and Henikoff and Henikoff, Proc. Natl. Acad. Sci. USA 89:10915-19, 1992. Briefly, two amino acid sequences are aligned to optimize the alignment scores using a gap opening penalty of 10, a gap extension penalty of 1, and the “blosum 62” scoring matrix of Henikoff and Henikoff (ibid.) as shown below (amino acids are indicated by the standard one-letter codes).

The “percent sequence identity” between two or more nucleic acid or amino acid sequences is a function of the number of identical positions shared by the sequences. Thus, % identity may be calculated as the number of identical nucleotides / amino acids divided by the total number of nucleotides / amino acids, multiplied by 100. Calculations of % sequence identity may also take into account the number of gaps, and the length of each gap that needs to be introduced to optimize alignment of two or more sequences. Sequence comparisons and the determination of percent identity between two or more sequences can be carried out using specific mathematical algorithms, such as BLAST, which will be familiar to a skilled person.

### ALIGNMENT SCORES FOR DETERMINING SEQUENCE IDENTITY

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 4 | | | | | | | | | | | | | | | | | | | |
| R | -1 | 5 | | | | | | | | | | | | | | | | | | |
| N | -2 | 0 | 6 | | | | | | | | | | | | | | | | | |
| D | -2 | -2 | 1 | 6 | | | | | | | | | | | | | | | | |
| C | 0 | -3 | -3 | -3 | 9 | | | | | | | | | | | | | | | |
| Q | -1 | 1 | 0 | 0 | -3 | 5 | | | | | | | | | | | | | | |
| E | -1 | 0 | 0 | 2 | -4 | 2 | 5 | | | | | | | | | | | | | |
| G | 0 | -2 | 0 | -1 | -3 | -2 | -2 | 6 | | | | | | | | | | | | |
| H | -2 | 0 | 1 | -1 | -3 | 0 | 0 | -2 | 8 | | | | | | | | | | | |
| I | -1 | -3 | -3 | -3 | -1 | -3 | -3 | -4 | -3 | 4 | | | | | | | | | | |
| L | -1 | -2 | -3 | -4 | -1 | -2 | -3 | -4 | -3 | 2 | 4 | | | | | | | | | |
| K | -1 | 2 | 0 | -1 | -3 | 1 | 1 | -2 | -1 | -3 | -2 | 5 | | | | | | | | |
| M | -1 | -1 | -2 | -3 | -1 | 0 | -2 | -3 | -2 | 1 | 2 | -1 | 5 | | | | | | | |
| F | -2 | -3 | -3 | -3 | -2 | -3 | -3 | -3 | -1 | 0 | 0 | -3 | 0 | 6 | | | | | | |
| P | -1 | -2 | -2 | -1 | -3 | -1 | -1 | -2 | -2 | -3 | -3 | -1 | -2 | -4 | 7 | | | | | |
| S | 1 | -1 | 1 | 0 | -1 | 0 | 0 | 0 | -1 | -2 | -2 | 0 | -1 | -2 | -1 | 4 | | | | |
| T | 0 | -1 | 0 | -1 | -1 | -1 | -1 | -2 | -2 | -1 | -1 | -1 | -1 | -2 | -1 | 1 | 5 | | | |
| W | -3 | -3 | -4 | -4 | -2 | -2 | -3 | -2 | -2 | -3 | -2 | -3 | -1 | 1 | -4 | -3 | -2 | 11 | | |
| Y | -2 | -2 | -2 | -3 | -2 | -1 | -2 | -3 | 2 | -1 | -1 | -2 | -1 | 3 | -3 | -2 | -2 | 2 | 7 | |
| V | 0 | -3 | -3 | -3 | -1 | -2 | -2 | -3 | -3 | 3 | 1 | -2 | 1 | -1 | -2 | -2 | 0 | -3 | -1 | 4 |

The percent identity is then calculated as:

$$\frac{\text{Total number of identical matches}}{\begin{matrix}[ \text{length of the longer sequence plus the} \\ \text{number of gaps introduced into the longer} \\ \text{sequence in order to align the two sequences} ]\end{matrix}} \times 100$$

Substantially homologous polypeptides are characterized as having one or more amino acid substitutions, deletions or additions. These changes are preferably of a minor nature, that is conservative amino acid substitutions (see below) and other substitutions that do not significantly affect the folding or activity of the polypeptide; small deletions, typically of one to about 30 amino acids; and small amino- or carboxyl-terminal extensions, such as an amino-terminal methionine residue, a small linker peptide of up to about 20-25 residues, or an affinity tag.

CONSERVATIVE AMINO ACID SUBSTITUTIONS

| | |
|---|---|
| Basic: | arginine |
| | lysine |
| | histidine |
| Acidic: | glutamic acid |
| | aspartic acid |
| Polar: | glutamine |
| | asparagine |
| Hydrophobic: | leucine |
| | isoleucine |
| | valine |
| Aromatic: | phenylalanine |
| | tryptophan |
| | tyrosine |
| Small: | glycine |
| | alanine |
| | serine |
| | methionine |

In addition to the 20 standard amino acids, non-standard amino acids (such as 4-hydroxyproline, 6-N-methyl lysine, 2-aminoisobutyric acid, isovaline and α -methyl serine) may be substituted for amino acid residues of the polypeptides of the present invention. A limited number of non-conservative amino acids, amino acids that are not encoded by the genetic code, and unnatural amino acids may be substituted for polypeptide amino acid residues. The polypeptides of the present invention can also comprise non-naturally occurring amino acid residues.

Non-naturally occurring amino acids include, without limitation, trans-3-methylproline, 2,4-methano-proline, cis-4-hydroxyproline, trans-4-hydroxy-proline, N-methylglycine, allo-threonine, methyl-threonine, hydroxy-ethylcysteine, hydroxyethylhomo-cysteine, nitro-glutamine, homoglutamine, pipecolic acid, tert-leucine, norvaline, 2-azaphenylalanine, 3-azaphenyl-alanine, 4-azaphenyl-alanine, and 4-fluorophenylalanine. Several methods are known in the art for incorporating non-naturally occurring amino acid residues into proteins. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is carried out in a cell free system comprising an E. coli S30 extract and commercially available enzymes and other reagents. Proteins are purified by chromatography. See, for example, Robertson et al., J. Am. Chem. Soc. 113:2722, 1991; Ellman et al., Methods Enzymol. 202:301, 1991; Chung et al., Science 259:806-9, 1993; and Chung et al., Proc. Natl. Acad. Sci. USA 90:10145-9, 1993). In a second method, translation is carried out in Xenopus oocytes by microinjection of mutated mRNA and chemically aminoacylated suppressor tRNAs (Turcatti et al., J. Biol. Chem. 271:19991-8, 1996). Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the polypeptide in place of its natural counterpart. See, Koide et al., Biochem. 33:7470-6, 1994. Naturally occurring amino acid residues can be converted to non-naturally occurring species by in vitro chemical modification. Chemical modification can be combined with site-directed mutagenesis to further expand the range of substitutions (Wynn and Richards, Protein Sci. 2:395-403, 1993).

A limited number of non-conservative amino acids, amino acids that are not encoded by the genetic code, non-naturally occurring amino acids, and unnatural amino acids may be substituted for amino acid residues of polypeptides of the present invention.

Essential amino acids in the polypeptides of the present invention can be identified according to procedures known in the art, such as site-directed mutagenesis or alanine-scanning mutagenesis (Cunningham and Wells, Science 244: 1081-5, 1989). Sites of biological interaction can also be determined by physical analysis of structure, as determined by such techniques as nuclear magnetic resonance, crystallography, electron diffraction or photoaffinity labeling, in conjunction with mutation of putative contact site amino acids. See, for example, de Vos et al., Science 255:306-12, 1992; Smith et al., J. Mol. Biol. 224:899-904, 1992; Wlodaver et al., FEBS Lett. 309:59-64, 1992. The identities of essential amino acids can also be inferred from analysis of homologies with related components (e.g. the translocation or protease components) of the polypeptides of the present invention.

Multiple amino acid substitutions can be made and tested using known methods of mutagenesis and screening, such as those disclosed by Reidhaar-Olson and Sauer (Science 241:53-7, 1988) or Bowie and Sauer (Proc. Natl. Acad. Sci. USA 86:2152-6, 1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832-7, 1991; Ladner et al., U.S. Patent No. 5,223,409; Huse, WIPO Publication WO 92/06204) and region-directed mutagenesis (Derbyshire et al., Gene 46:145, 1986; Ner et al., DNA 7:127, 1988).

Unless defined otherwise, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 20 ED., John Wiley and Sons, New York (1994), and Hale & Marham, THE HARPER COLLINS DICTIONARY OF BIOLOGY, Harper Perennial, NY (1991) provide the skilled person with a general dictionary of many of the terms used in this disclosure.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

The headings provided herein are not limitations of the various aspects or embodiments of this disclosure.

Amino acids are referred to herein using the name of the amino acid, the three letter abbreviation or the single letter abbreviation. The term “protein”, as used herein, includes proteins, polypeptides, and peptides. As used herein, the term “amino acid sequence” is synonymous with the term “polypeptide” and/or the term “protein”. In some instances, the term “amino acid sequence” is synonymous with the term “peptide”. In some instances, the term “amino acid sequence” is synonymous with the term “enzyme”. The terms “protein” and “polypeptide” are used interchangeably herein. In the present disclosure and claims, the conventional one-letter and three-letter codes for amino acid residues may be used. The 3-letter code for amino acids as defined in conformity with the IUPACIUB Joint Commission on Biochemical Nomenclature (JCBN). It is also understood that a polypeptide may be coded for by more than one nucleotide sequence due to the degeneracy of the genetic code.

Other definitions of terms may appear throughout the specification. Before the exemplary embodiments are described in more detail, it is to be understood that this disclosure is not limited to particular embodiments described, and as such may vary. It is also to be understood that the terminology used herein is for the purpose of describing particular embodiments only, and is not intended to be limiting, since the scope of the present disclosure will be defined only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include plural referents unless the context clearly dictates otherwise. Thus, for example, reference to “a granulocyte” includes a plurality of such candidate agents and reference to “the granulocyte” includes reference to one or more granulocytes and equivalents thereof known to those skilled in the art, and so forth.

The publications discussed herein are provided solely for their disclosure prior to the filing date of the present application. Nothing herein is to be construed as an admission that such publications constitute prior art to the claims appended hereto.

### BRIEF DESCRIPTION OF THE DRAWINGS

Embodiments of the invention will now be described, by way of example only, with reference to the following Figures and Examples.

FIG. 1 shows the distribution of different types of neutrophils in the general population. Defective neutrophils are characterised by an anti-inflammatory and proangiogenic phenotype whereas healthy neutrophils are characterised by a proinflammatory and antiangiogenic phenotype. Finally, exceptional neutrophils are present in a proportion of the general population and exhibit the extreme of all qualities found in healthy neutrophils.

FIG. 2 shows significantly (***P < 0.001, **P < 0.01, *P < 0.05) upregulated expression of ITGB1, SYK, DOCK8 and CYBB in high CKA neutrophils (>80% CKA) compared with low CKA control neutrophils. Data are mean (n = 3 donor samples in duplicate) ± standard error of mean compared by two-way ANOVA.

FIG. 3 shows significantly (**P < 0.01, *P < 0.05) upregulated expression of PLEC and COMP in high CKA neutrophils (>80% CKA) compared with low CKA control neutrophils. Data are mean (n = 3 donor samples in duplicate) ± standard error of mean compared by two-way ANOVA.

FIG. 4 shows significantly (**P < 0.01, *P < 0.05) upregulated expression of ATG7, SLC2A1, S100A9, ACSL1, CTSG, PSMB2, ATM and S100A8 in high CKA neutrophils (>80% CKA) compared with low CKA control neutrophils. Data are mean (n = 3 donor samples in duplicate) ± standard error of mean compared by two-way ANOVA.

FIG. 5 shows significantly (**P < 0.01, *P < 0.05) downregulated expression of ANXA1 in high CKA neutrophils (>80% CKA) compared with low CKA control neutrophils. Data are mean (n = 3 donor samples in duplicate) ± standard error of mean compared by two-way ANOVA.

FIG. 6 shows significantly (**P < 0.01, *P < 0.05) upregulated expression of BCAP31 and TAPBP in high CKA neutrophils (>80% CKA) compared with low CKA control neutrophils. Data are mean (n = 3 donor samples in duplicate) ± standard error of mean compared by two-way ANOVA.

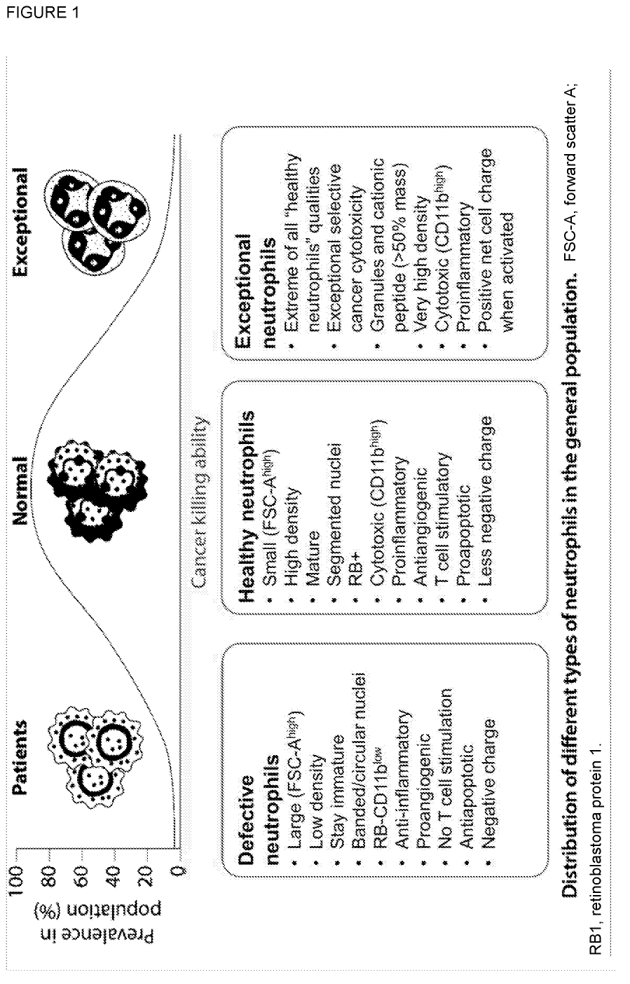

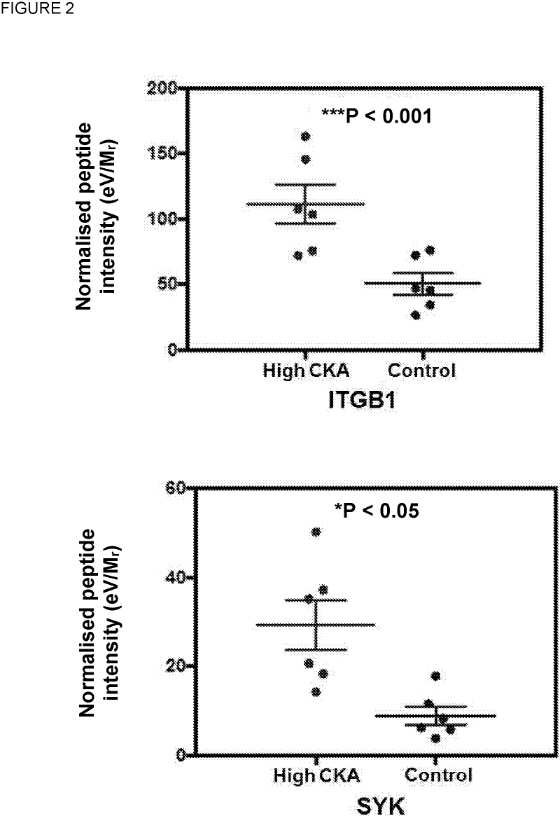

### SEQUENCE LISTING

Gene Sequences

| SEQ ID NO. | GENE | Accession No. | Ensembl Release No. |
|---|---|---|---|
| 1 | CTSG | ENSG00000100448 | 97 |
| 2 | CAP37 | ENSG00000172232 | 97 |
| 3 | ITGB1 | ENSG00000150093 | 97 |
| 4 | CYBB | ENSG00000165168 | 97 |
| 5 | SYK | ENSG00000165025 | 97 |
| 6 | DOCK8 | ENSG00000107099 | 97 |
| 7 | COMP | ENSG00000105664 | 97 |
| 8 | ATG7 | ENSG00000197548 | 97 |
| 9 | SLC2A1 | ENSG00000117394 | 97 |
| 10 | GZMK | ENSG00000113088 | 97 |
| 11 | S100A9 | ENSG00000163220 | 97 |
| 12 | S100A8 | ENSG00000143546 | 97 |
| 13 | ATM | ENSG00000149311 | 97 |
| 14 | IKBKB | ENSG00000104365 | 97 |
| 15 | BCAP31 | ENSG00000185825 | 97 |
| 16 | TAPBP | ENSG00000231925 | 97 |
| 17 | PPP3CB | ENSG00000107758 | 97 |
| 18 | ANXA1 | ENSG00000135046 | 97 |
| 19 | PERM | ENSG00000005381 | 97 |
| 20 | PLEC | ENSG00000178209 | 97 |
| 21 | ACSL1 | ENSG00000151726 | 97 |
| 22 | RAC1 | ENSG00000136238 | 97 |
| 23 | PSMB2 | ENSG00000126067 | 97 |

### POLYPEPTIDE SEQUENCES

| | | | | | | |
|---|---|---|---|---|---|---|
| SEQ ID NO. | POLYPEPTIDE | ISOFORM | UniProt ID | UniProt Version Number | SEQUENCE | |
| 25 | CTSG | 1 | P08311 | Entry version 191 (18 Sep. 2019) Sequence version 2 (01 Jan. 1990) | MQPLLLLLAF IIGGRESRPH QSPAGQSRCG TAAHCWGSNI RRENTQQHIT NQRTIQNDIM NRNVNPVALP LCTVAGWGRV EVQLRVQRDR PRRQICVGDR SGGPLLCNNV AHGIVSYGKS TMRSFKLLDQ | LLPTGAEAGE SRPYMAYLQI GFLVREDFVL NVTLGAHNIQ ARRAIRHPQY LLQLSRRVRR RAQEGLRPGT SMRRGTDTLR QCLRIFGSYD RERKAAFKGD SGVPPEVFTR VSSFLPWIRT METPL |
| 26 | CAP37 | 1 | P20160 | Entry version 191 (18 Sep. 2019) Sequence version 3 (01 Oct. 1993) | MTRLTVLALL SSPLLDIVGG LASIQNQGRH FVMTAASCFQ LGAYDLRRRE SMSENGYDPQ LDREANLTSS ATVEAGTRCQ GRLSRFPRFV RPNNVCTGVL GGTPLVCEGL PCGRGPDFFT GVLNNPGPGP | AGLLASSRAG RKARPRQFPF FCGGALIHAR SQNPGVSTVV RQSRQTFSIS QNLNDLMLLQ VTILPLPLQN VAGWGSQRSG NVTVTPEDQC TRRGGICNGD AHGVASFSLG RVALFRDWID A |
| 27 | ITGB1 | 1 | P05556 | Entry version 242 (18 Sep. 2019) Sequence version 2 (16 Dec. 2008) | MNLQPIFWIG QTDENRCLKA AGPNCGWCTN SARCDDLEAL ENPRGSKDIK GTAEKLKPED LRLRSGEPQT YPIDLYYLMD NVKSLGTDLM RIGFGSFVEK | LISSVCCVFA NAKSCGECIQ STFLQEGMPT KKKGCPPDDI KNKNVTNRSK ITQIQPQQLV FTLKFKRAED LSYSMKDDLE NEMRRITSDF TVMPYISTTP |
| 27 | ITGB1 | 1 | P05556 | Entry version 242 (18 Sep. 2019) Sequence version 2 (16 Dec. 2008) | MNLQPIFWIG QTDENRCLKA AGPNCGWCTN SARCDDLEAL ENPRGSKDIK GTAEKLKPED LRLRSGEPQT YPIDLYYLMD NVKSLGTDLM RIGFGSFVEK | LISSVCCVFA NAKSCGECIQ STFLQEGMPT KKKGCPPDDI KNKNVTNRSK ITQIQPQQLV FTLKFKRAED LSYSMKDDLE NEMRRITSDF TVMPYISTTP |
| | | | | | AKLRNPCTSE NVLSLTNKGE ISGNLDSPEG CGSLIGWRNV GFHFAGDGKL CHLENNMYTM HLVQKLSENN FQPVYKELKN SANSSNVIQL EVILENGKLS CKNGVNGTGE GDEVQFEISI DSFKIRPLGF ICECECQSEG NGTFECGACR ECSTDEVNSE SSEICSNNGE RDNTNEIYSG DRSNGLICGG CNPNYTGSAC ASNGQICNGR TDPKFQGQTC AEHKECVQCR CTQECSYFNI QPVQPDPVSH FYFTYSVNGN PECPTGPDII VLIGLALLLI REFAKFEKEK PIYKSAVTTV | QNCTSPFSYK VFNELVGKQR GFDAIMQVAV TRLLVFSTDA GGIVLPNDGQ SHYYDYPSIA IQTIFAVTEE LIPKSAVGTL IIDAYNSLSS EGVTISYKSY NGRKCSNISI TSNKCPKKDS TEEVEVILQY IPESPKCHEG CNEGRVGRHC DMDAYCRKEN CVCGQCVCRK KFCECDNFNC NGVCKCRVCE DCSLDTSTCE GICECGVCKC EMCQTCLGVC AFNKGEKKDT TKVESRDKLP CKEKDVDDCW NEVMVHVVEN PIVAGVVAGI WKLLMIIHDR MNAKWDTGEN VNPKYEGK |
| 28 | ITGB1 | 2 | P055562 | Entry version 242 (18 Sep. 2019) Sequence version 2 (16 Dec. 2008) | MNLQPIFWIG QTDENRCLKA AGPNCGWCTN SARCDDLEAL ENPRGSKDIK GTAEKLKPED LRLRSGEPQT YPIDLYYLMD NVKSLGTDLM RIGFGSFVEK AKLRNPCTSE NVLSLTNKGE ISGNLDSPEG CGSLIGWRNV GFHFAGDGKL CHLENNMYTM HLVQKLSENN FQPVYKELKN SANSSNVIQL EVILENGKLS CKNGVNGTGE GDEVQFEISI DSFKIRPLGF ICECECQSEG NGTFECGACR ECSTDEVNSE SSEICSNNGE RDNTNEIYSG DRSNGLICGG | LISSVCCVFA NAKSCGECIQ STFLQEGMPT KKKGCPPDDI KNKNVTNRSK ITQIQPQQLV FTLKFKRAED LSYSMKDDLE NEMRRITSDF TVMPYISTTP QNCTSPFSYK VFNELVGKQR GFDAIMQVAV TRLLVFSTDA GGIVLPNDGQ SHYYDYPSIA IQTIFAVTEE LIPKSAVGTL IIDAYNSLSS EGVTISYKSY NGRKCSNISI TSNKCPKKDS TEEVEVILQY IPESPKCHEG CNEGRVGRHC DMDAYCRKEN CVCGQCVCRK KFCECDNFNC NGVCKCRVCE |
| | | | | | CNPNYTGSAC ASNGQICNGR TDPKFQGQTC AEHKECVQCR CTQECSYFNI QPVQPDPVSH FYFTYSVNGN PECPTGPDII VLIGLALLLI REFAKFEKEK KTSKKQSGL | DCSLDTSTCE GICECGVCKC EMCQTCLGVC AFNKGEKKDT TKVESRDKLP CKEKDVDDCW NEVMVHVVEN PIVAGVVAGI WKLLMIIHDR MNAKWDTVSY |
| 29 | ITGB1 | 3 | P055563 | Entry version 242 (18 Sep. 2019) Sequence version 2 (16 Dec. 2008) | MNLQPIFWIG QTDENRCLKA AGPNCGWCTN SARCDDLEAL ENPRGSKDIK GTAEKLKPED LRLRSGEPQT YPIDLYYLMD NVKSLGTDLM RIGFGSFVEK AKLRNPCTSE NVLSLTNKGE ISGNLDSPEG CGSLIGWRNV GFHFAGDGKL CHLENNMYTM HLVQKLSENN FQPVYKELKN SANSSNVIQL EVILENGKLS CKNGVNGTGE GDEVQFEISI DSFKIRPLGF ICECECQSEG NGTFECGACR ECSTDEVNSE SSEICSNNGE RDNTNEIYSG DRSNGLICGG CNPNYTGSAC ASNGQICNGR TDPKFQGQTC AEHKECVQCR CTQECSYFNI QPVQPDPVSH FYFTYSVNGN PECPTGPDII VLIGLALLLI REFAKFEKEK VAQPGVQWCD FQQFSCLSLP FIRVP | LISSVCCVFA NAKSCGECIQ STFLQEGMPT KKKGCPPDDI KNKNVTNRSK ITQIQPQQLV FTLKFKRAED LSYSMKDDLE NEMRRITSDF TVMPYISTTP QNCTSPFSYK VFNELVGKQR GFDAIMQVAV TRLLVFSTDA GGIVLPNDGQ SHYYDYPSIA IQTIFAVTEE LIPKSAVGTL IIDAYNSLSS EGVTISYKSY NGRKCSNISI TSNKCPKKDS TEEVEVILQY IPESPKCHEG CNEGRVGRHC DMDAYCRKEN CVCGQCVCRK KFCECDNFNC NGVCKCRVCE DCSLDTSTCE GICECGVCKC EMCQTCLGVC AFNKGEKKDT TKVESRDKLP CKEKDVDDCW NEVMVHVVEN PIVAGVVAGI WKLLMIIHDR MNAKWDTSLS ISSLQPLTSR STWDYRVKIL |
| 30 | ITGB1 | 4 | P055564 | Entry version 242 (18 Sep. 2019) Sequence version 2 (16 Dec. 2008) | MNLQPIFWIG QTDENRCLKA AGPNCGWCTN SARCDDLEAL ENPRGSKDIK GTAEKLKPED | LISSVCCVFA NAKSCGECIQ STFLQEGMPT KKKGCPPDDI KNKNVTNRSK ITQIQPQQLV |
| | | | | | LRLRSGEPQT YPIDLYYLMD NVKSLGTDLM RIGFGSFVEK AKLRNPCTSE NVLSLTNKGE ISGNLDSPEG CGSLIGWRNV GFHFAGDGKL CHLENNMYTM HLVQKLSENN FQPVYKELKN SANSSNVIQL EVILENGKLS CKNGVNGTGE GDEVQFEISI DSFKIRPLGF ICECECQSEG NGTFECGACR ECSTDEVNSE SSEICSNNGE RDNTNEIYSG DRSNGLICGG CNPNYTGSAC ASNGQICNGR TDPKFQGQTC AEHKECVQCR CTQECSYFNI QPVQPDPVSH FYFTYSVNGN PECPTGPDII VLIGLALLLI REFAKFEKEK QWCDISSLQP LSLPSTWDYR | FTLKFKRAED LSYSMKDDLE NEMRRITSDF TVMPYISTTP QNCTSPFSYK VFNELVGKQR GFDAIMQVAV TRLLVFSTDA GGIVLPNDGQ SHYYDYPSIA IQTIFAVTEE LIPKSAVGTL IIDAYNSLSS EGVTISYKSY NGRKCSNISI TSNKCPKKDS TEEVEVILQY IPESPKCHEG CNEGRVGRHC DMDAYCRKEN CVCGQCVCRK KFCECDNFNC NGVCKCRVCE DCSLDTSTCE GICECGVCKC EMCQTCLGVC AFNKGEKKDT TKVESRDKLP CKEKDVDDCW NEVMVHVVEN PIVAGVVAGI WKLLMIIHDR MNAKWDTPGV LTSRFQQFSC VKILFIRVP |
| 31 | ITGB1 | 5 | P055565 | Entry version 242 (18 Sep. 2019) Sequence version 2 (16 Dec. 2008) | MNLQPIFWIG QTDENRCLKA AGPNCGWCTN SARCDDLEAL ENPRGSKDIK GTAEKLKPED LRLRSGEPQT YPIDLYYLMD NVKSLGTDLM RIGFGSFVEK AKLRNPCTSE NVLSLTNKGE ISGNLDSPEG CGSLIGWRNV GFHFAGDGKL CHLENNMYTM HLVQKLSENN FQPVYKELKN SANSSNVIQL EVILENGKLS CKNGVNGTGE GDEVQFEISI DSFKIRPLGF ICECECQSEG NGTFECGACR | LISSVCCVFA NAKSCGECIQ STFLQEGMPT KKKGCPPDDI KNKNVTNRSK ITQIQPQQLV FTLKFKRAED LSYSMKDDLE NEMRRITSDF TVMPYISTTP QNCTSPFSYK VFNELVGKQR GFDAIMQVAV TRLLVFSTDA GGIVLPNDGQ SHYYDYPSIA IQTIFAVTEE LIPKSAVGTL IIDAYNSLSS EGVTISYKSY NGRKCSNISI TSNKCPKKDS TEEVEVILQY IPESPKCHEG CNEGRVGRHC |
| | | | | | ECSTDEVNSE SSEICSNNGE RDNTNEIYSG DRSNGLICGG CNPNYTGSAC ASNGQICNGR TDPKFQGQTC AEHKECVQCR CTQECSYFNI QPVQPDPVSH FYFTYSVNGN PECPTGPDII VLIGLALLLI REFAKFEKEK PIYKSPINNF L | DMDAYCRKEN CVCGQCVCRK KFCECDNFNC NGVCKCRVCE DCSLDTSTCE GICECGVCKC EMCQTCLGVC AFNKGEKKDT TKVESRDKLP CKEKDVDDCW NEVMVHVVEN PIVAGVVAGI WKLLMIIHDR MNAKWDTQEN KNPNYGRKAG |
| 32 | CYBB | 1 | P04839 | Entry version 213 (18 Sep. 2019) Sequence version 2 (23 Jan. 2007) | MGNWAVNEGL LNVFLFVWYY YTRKLLGSAL NFNCMLILLP GSSACCSTRV HKMVAWMIAL FNVEWCVNAR LSELGDRQNE KNPEGGLYLA VITLCLILII YFEVFWYTHH IHGAERIVRG ITVCEQKISE QFAGNPPMTW LCERLVRFWR VTHPFKTIEL VGQYIFVKCP TLTSAPEEDF WTEGLFNACG KLPKIAVDGP YEVVMLVGAG KSVWYKYCNN FYWLCRDTHA LESQMQERNN TGWDESQANH VITGLKQKTL KTIASQHPNT ALAETLSKQS VHFIFNKENF | SIFVILVWLG RVYDIPPKFF ALARAPAACL VCRNLLSFLR RRQLDRNLTF HSAIHTIAHL VNNSDPYSVA SYLNFARKRI VTLLAGITGV TSSTKTIRRS LFVIFFIGLA QTAESLAVHN WGKIKECPIP KWIVGPMFLY SQQKWITKV QMKKKGFKME KVSKLEWHPF FSIHIRIVGD CDKQEFQDAW FGTASEDVFS IGVTPFASIL ATNLKLKKIY FEWFADLLQL AGFLSYNIYL FAVHHDEEKD YGRPNWDNEF RIGVFLCGPE ISNSESGPRG |
| 33 | SYK | 1 | P43405 | Entry version 224 (18 Sep. 2019) Sequence version 1 (01 Nov. 1995) | MASSGMADSA TREEAEDYLV LRQSRNYLGG AHHYTIEREL THASPADLCH CLLKKPFNRP EDLKENLIRE QALEQAIISQ TAHEKMPWFH IVLIGSKTNG GSYALCLLHE DKTGKLSIPE VEHYSYKADG | NHLPFFFGNI QGGMSDGLYL FALSVAHGRK NGTYAIAGGR YHSQESDGLV QGVQPKTGPF YVKQTWNLQG KPQLEKLIAT GKISREESEQ KFLIRARDNN GKVLHYRIDK GKKFDTLWQL LLRVLTVPCQ |
| | | | | | KIGTQGNVNF PATWSAGGII PGHRKSSPAQ NPYEPELAPW LPMDTEVYES KEVYLDRKLL NFGTVKKGYY VKILKNEAND ANVMQQLDNP AESWMLVMEM QQNRHVKDKN GMKYLEESNF LLVTQHYAKI ADENYYKAQT PECINYYKFS LMWEAFSYGQ VTAMLEKGER MYDLMNLCWT AVELRLRNYY | GGRPQLPGSH SRIKSYSFPK GNRQESTVSF AADKGPQREA PYADPEEIRP TLEDKELGSG QMKKVVKTVA PALKDELLAE YIVRMIGICE AELGPLNKYL IIELVHQVSM VHRDLAARNV SDFGLSKALR HGKWPVKWYA SKSDVWSFGV KPYRGMKGSE MGCPAGCPRE YDVENRPGFA YDVVN |
| 34 | SYK | 2 | P434052 | Entry version 224 (18 Sep. 2019) Sequence version 1 (01 Nov. 1995) | MASSGMADSA TREEAEDYLV LRQSRNYLGG AHHYTIEREL THASPADLCH CLLKKPFNRP EDLKENLIRE QALEQAIISQ TAHEKMPWFH IVLIGSKTNG GSYALCLLHE DKTGKLSIPE VEHYSYKADG KIGTQGNVNF PASSPAQGNR EPELAPWAAD DTEVYESPYA YLDRKLLTLE TVKKGYYQMK LKNEANDPAL MQQLDNPYIV WMLVMEMAEL RHVKDKNIIE YLEESNFVHR TQHYAKISDF NYYKAQTHGK INYYKFSSKS EAFSYGQKPY MLEKGERMGC LMNLCWTYDV LRLRNYYYDV | NHLPFFFGNI QGGMSDGLYL FALSVAHGRK NGTYAIAGGR YHSQESDGLV QGVQPKTGPF YVKQTWNLQG KPQLEKLIAT GKISREESEQ KFLIRARDNN GKVLHYRIDK GKKFDTLWQL LLRVLTVPCQ GGRPQLPGSH QESTVSFNPY KGPQREALPM DPEEIRPKEV DKELGSGNFG KVVKTVAVKI KDELLAEANV RMIGICEAES GPLNKYLQQN LVHQVSMGMK DLAARNVLLV GLSKALRADE WPVKWYAPEC DVWSFGVLMW RGMKGSEVTA PAGCPREMYD ENRPGFAAVE VN |
| 35 | DOCK8 | 1 | Q8NF50 | Entry version 160 (18 Sep. 2019) Sequence version 3 (21 Aug. 2007) | MATLPSAERR SAEIRKQFTL SISTSGFPSL PVDFEGLLMT QELGDFTDDD RTLQPSLPEE CVQTYIREWL EICGFKKTGS | AFALKINRYS PPNLGQYHRQ QLPQFYDPVE HLNSLDVQLA LDVVFTPKEC GVELDPHVRD IVNRKNQGSP RKDFHKTLPK |
| | | | | | QTFESETLEC HLNVLCDVSG LRSLQPDKRL DFEKQNEEAR LYPSVDEEDA KEHLGNRILV IEPLFASIAL ENFHCDLNSD PSVAASSQAR SDIYLVVKIE CAEPYTVIKE EKLKLQAESF FAWAPISLSS VTDVDSVVGR QSRRLSERAL FKTSTLSVSS DEDLFKFLAD VKSIPGLLRL CCLTPEMLPV HKEILEFPTR NLLYVYPQRL NITIKIQFMC IFGKSSGPEF HNKSPDFYEE VNHHLLFTFY SVETLLGYSW TGSYCLPVAL SAEKVPLQNP VFNIEVQAVS KFFTLCHSLE DQKISEMALE NSSRLEPLVL QLSVQPMVIA FESVVAIANS QHGRNCLLAS VQRDVPKSGA HTYGRTSAAA MSSSNPDLAG NIMSSKIADR GSSDAPSSPA HEELALQMW KYAWFFFELL MDKRDSFRRT TTIVNVVTSE ENEQAEKMNI SLMDRGFVFN AKLSNLPTLI CSHEHYLNLN TSPCPSISSQ QKIASMFDLT GLLFTELAAA VQRKAVSAIH RCVKPEVKVK IILDALPQLC RTSGSDEEQE LAIAGNNFNL PYKQYNMLNA LWIMKNADQS STQLNRILDL KGKQSSDKVS | SEPAAQAGPR KGPVTACDFD ENLLQQVSAE RTNRQAELFA VEIRPVPECP KLLTLKFEIE YDVKERKKIS QFKGFLRAHT SAVFSVTYPS KVLQQGEIGD SDGGKSKEKI CQRLGKYRMP FFNVSTLERE SSVGERRTLA SLEENGVGSN FFKQEGDRLS YKRSSSLQRR EISTAPEIIN KPFPENRTRP EVYVPHTVYR NFVNKLASAR GEDASNAMPV LQEVYTAVTY VKIKLPAKLT HISCQQKQGA LPILLNERLQ EKLPPNYSMH PIKWAEGHKG SVHTQDNHLE SQVTFPIRVL HELKLSIICL FLHLVLDKLF GQTANFSQFA LHNSKDLSKD YVHYVFRLPE PTALLDPRSY VSSKLLQARV THSAADEEVK NCSRMSYYCS APRPASKKHF STGMVRETVF VKSMAQHVHN RFSDRFMDDI IAALLVKPQK SLAFFLYDLL LIRHYCSQLS SMRLEFLRIL LFFMNADTAP NSSSCSSFQD SEYRQQHFLT LDAEGEGISK SLLSSHDLDP IAALYLPLVG DFTVADTRRY GAGAINQNVA KTSGIVLSSL DTTRNLMICF LIRKWIADLP LFICVLCFEY TQVLQKSRDV |
| | | | | | KARLEEALLR EQTHWRQANE QEALISGNLA QENIIQASSA VLRVLVNSLN CFATLRALIA VEQCFDLCHQ VTRSQACATL TSNFARVKMQ RAPDFNEEHL SEEDTAMQMT CNLNSILYDT EMLMDLMYRI RLTWLQNMAE AAMCLVHAAA DHSYLPVGSV EESVVSEDTL QYFTESGLVG TGGLYETVNE AHREFRKLTL SIVNKDHKRM GSKFGDLDEQ KLPEISHRLE FVEVIKDSTP AYIQITFVEP YFDEYEMKDR VTYFEKNFNL LEGRPRGELH TMHAFPYIKT VLTPIEVAIE VAINQEPPDA VGATVNQGPL PADPKLYRHH IMRCGEAVEK EYQQELKKNY IERKIPELYK DSFHRSSFRK | GEGARGEMMR RRAPGNDRFP GLNENLRWKK KLDKTKAELD TEAHLIILDM LDCKDSLLGG CDQSTTYLTH KFGDLLFEEE VLHHCSSSMD YLLMRFSFGA VTMSLASLVG RRSLRTILAY PFPTQVEELL VKMREFQEDP AKSYQASPDL KHTKKKCYTE LVAEYLSMLE SFQNISSNVL SPDEDGVCAG LLEQAAELFS VYKLVIPILE THSKLQRAFD FGTYFRVGFF EFVYKEPAIT AFYGQCFGAE VDKTKLDPNK RRFMYTTPFT EQYRRNTVLT RISVIQKEEF DMKKKTLQLA KMLQMVLQGS EVAQVFLAEI NKLRLCFKEF NKRLITADQR NKLKENLRPM PIFRVESQKR CETQLSQGS |
| 36 | DOCK8 | 2 | Q8NF502 | Entry version 160 (18 Sep. 2019) Sequence version 3 (21 Aug. 2007) | MATLPSAERR SAEIRKQFTL SISTSGFPSL PVDFEGLLMT QELGDFTDDD RTLQPSLPEE CVQTYIREWL EICGFKKTGS QTFESETLEC HLNVLCDVSG LRSLQPDKRL DFEKQNEEAR LYPSVDEEDA KEHLGNRILV IEPLFASIAL ENFHCDLNSD PSVAASSQAR SDIYLVVKIE CAEPYTVIKE EKLKLQAESF FAWAPISLSS VTDVDSVVGR | AFALKINRYS PPNLGQYHRQ QLPQFYDPVE HLNSLDVQLA LDVVFTPKEC GVELDPHVRD IVNRKNQGSP RKDFHKTLPK SEPAAQAGPR KGPVTACDFD ENLLQQVSAE RTNRQAELFA VEIRPVPECP KLLTLKFEIE YDVKERKKIS QFKGFLRAHT SAVFSVTYPS KVLQQGEIGD SDGGKSKEKI CQRLGKYRMP FFNVSTLERE SSVGERRTLA |
| | | | | | QSRRLSERAL FKTSTLSVSS DEDLFKFLAD VKSIPGLLRL CCLTPEMLPV HKEILEFPTR NLLYVYPQRL NITIKIQFMC IFGKSSGPEF HNKSPDFYEE VNHHLLFTFY SVETLLGYSW TGSYCLPVAL SAEKVPLQNP VFNIEVQAVS KFFTLCHSLE DQKISEMALE NSSRLEPLVL QLSVQPMVIA FESVVAIANS QHGRNCLLAS VQRDVPKSGA HTYGRTSAAA MSSSNPDLAG NIMSSKHFHE GMVRETVFKY SMAQHVHNMD SDRFMDDITT ALLVKPQKEN AFFLYDLLSL RHYCSQLSAK RLEFLRILCS FMNADTAPTS SSCSSFQDQK YRQQHFLTGL AEGEGISKVQ LSSHDLDPRC ALYLPLVGII TVADTRRYRT GAINQNVALA SGIVLSSLPY TRNLMICFLW RKWIADLPST ICVLCFEYKG VLQKSRDVKA GARGEMMRRR NENLRWKKEQ DKTKAELDQE AHLIILDMQE CKDSLLGGVL QSTTYLTHCF GDLLFEEEVE HHCSSSMDVT LMRFSFGATS MSLASLVGRA SLRTILAYSE PTQVEELLCN MREFQEDPEM SYQASPDLRL TKKKCYTEAA | SLEENGVGSN FFKQEGDRLS YKRSSSLQRR EISTAPEIIN KPFPENRTRP EVYVPHTVYR NFVNKLASAR GEDASNAMPV LQEVYTAVTY VKIKLPAKLT HISCQQKQGA LPILLNERLQ EKLPPNYSMH PIKWAEGHKG SVHTQDNHLE SQVTFPIRVL HELKLSIICL FLHLVLDKLF GQTANFSQFA LHNSKDLSKD YVHYVFRLPE PTALLDPRSY VSSKLLQARV THSAADEEVK ELALQMVVST AWFFFELLVK KRDSFRRTRF IVNVVTSEIA EQAEKMNISL MDRGFVFNLI LSNLPTLISM HEHYLNLNLF PCPSISSQNS IASMFDLTSE LFTELAAALD RKAVSAIHSL VKPEVKVKIA LDALPQLCDF SGSDEEQEGA IAGNNFNLKT KQYNMLNADT IMKNADQSLI QLNRILDLLF KQSSDKVSTQ RLEEALLRGE APGNDRFPGL THWRQANEKL ALISGNLATE NIIQASSALD RVLVNSLNCD ATLRALIAKF QCFDLCHQVL RSQACATLYL NFARVKMQVT PDFNEEHLRR EDTAMQMTPF LNSILYDTVK LMDLMYRIAK TWLQNMAEKH MCLVHAAALV |
| | | | | | AEYLSMLEDH QNISSNVLEE DEDGVCAGQY EQAAELFSTG KLVIPILEAH SKLQRAFDSI TYFRVGFFGS VYKEPAITKL YGQCFGAEFV KTKLDPNKAY DEYEMKDRVT FMYTTPFTLE YRRNTVLTTM SVIQKEEFVL KKKTLQLAVA LQMVLQGSVG AQVFLAEIPA LRLCFKEFIM RLITADQREY LKENLRPMIE FRVESQKRDS TQLSQGS | SYLPVGSVSF SVVSEDTLSP FTESGLVGLL GLYETVNEVY REFRKLTLTH VNKDHKRMFG KFGDLDEQEF PEISHRLEAF EVIKDSTPVD IQITFVEPYF YFEKNFNLRR GRPRGELHEQ HAFPYIKTRI TPIEVAIEDM INQEPPDAKM ATVNQGPLEV DPKLYRHHNK RCGEAVEKNK QQELKKNYNK RKIPELYKPI FHRSSFRKCE |
| 37 | DOCK8 | 3 | Q8NF503 | Entry version 160 (18 Sep. 2019) Sequence version 3 (21 Aug. 2007) | MTHLNSLDVQ DDLDVVFTPK EEGVELDPHV WLIVNRKNQG GSRKDFHKTL ECSEPAAQAG SGKGPVTACD RLENLLQQVS ARRTNRQAEL DAVEIRPVPE LVKLLTLKFE ALYDVKERKK SDQFKGFLRA ARSAVFSVTY IEKVLQQGEI KESDGGKSKE SFCQRLGKYR SSFFNVSTLE GRSSVGERRT ALSLEENGVG SSFFKQEGDR ADYKRSSSLQ RLEISTAPEI PVKPFPENRT TREVYVPHTV RLNFVNKLAS MCGEDASNAM EFLQEVYTAV EEVKIKLPAK FYHISCQQKQ SWLPILLNER ALEKLPPNYS NPPIKWAEGH VSSVHTQDNH LESQVTFPIR LEHELKLSII VLFLHLVLDK IAGQTANFSQ | LAQELGDFTD ECRTLQPSLP RDCVQTYIRE SPEICGFKKT PKQTFESETL PRHLNVLCDV FDLRSLQPDK AEDFEKQNEE FALYPSVDEE CPKEHLGNRI IEIEPLFASI ISENFHCDLN HTPSVAASSQ PSSDIYLVVK GDCAEPYTVI KIEKLKLQAE MPFAWAPISL REVTDVDSW LAQSRRLSER SNFKTSTLSV LSDEDLFKFL RRVKSIPGLL INCCLTPEML RPHKEILEFP YRNLLYVYPQ ARNITIKIQF PVIFGKSSGP TYHNKSPDFY LTVNHHLLFT GASVETLLGY LQTGSYCLPV MHSAEKVPLQ KGVFNIEVQA LEKFFTLCHS VLDQKISEMA CLNSSRLEPL LFQLSVQPMV FAFESVVAIA |
| | | | | | NSLHNSKDLS ASYVHYVFRL SYHTYGRTSA AAVSSKLLQA AGTHSAADEE DRNCSRMSYY PAAPRPASKK VVSTGMVRET LLVKSMAQHV RTRFSDRFMD SEIAALLVKP NISLAFFLYD FNLIRHYCSQ LISMRLEFLR LNLFFMNADT SQNSSSCSSF LTSEYRQQHF AALDAEGEGI IHSLLSSHDL VKIAALYLPL LCDFTVADTR QEGAGAINQN NLKTSGIVLS NADTTRNLMI QSLIRKWIAD DLLFICVLCF VSTQVLQKSR LRGEGARGEM FPGLNENLRW NEKLDKTKAE LATEAHLIIL SALDCKDSLL LNCDQSTTYL IAKFGDLLFE HQVLHHCSSS TLYLLMRFSF MQVTMSLASL HLRRSLRTIL MTPFPTQVEE DTVKMREFQE RIAKSYQASP AEKHTKKKCY AALVAEYLSM SVSFQNISSN TLSPDEDGVC VGLLEQAAEL NEVYKLVIPI TLTHSKLQRA RMFGTYFRVG EQEFVYKEPA LEAFYGQCFG TPVDKTKLDP EPYFDEYEMK NLRRFMYTTP LHEQYRRNTV KTRISVIQKE IEDMKKKTLQ DAKMLQMVLQ PLEVAQVFLA HHNKLRLCFK | KDQHGRNCLL PEVQRDVPKS GAPTALLDPR RVMSSSNPDL VKNIMSSKIA CSGSSDAPSS HFHEELALQM VFKYAWFFFE HNMDKRDSFR DITTIVNVVT QKENEQAEKM LLSLMDRGFV LSAKLSNLPT ILCSHEHYLN APTSPCPSIS QDQKIASMFD LTGLLFTELA SKVQRKAVSA DPRCVKPEVK VGIILDALPQ RYRTSGSDEE VALAIAGNNF SLPYKQYNML CFLWIMKNAD LPSTQLNRIL EYKGKQSSDK DVKARLEEAL MRRRAPGNDR KKEQTHWRQA LDQEALISGN DMQENIIQAS GGVLRVLVNS THCFATLRAL EEVEQCFDLC MDVTRSQACA GATSNFARVK VGRAPDFNEE AYSEEDTAMQ LLCNLNSILY DPEMLMDLMY DLRLTWLQNM TEAAMCLVHA LEDHSYLPVG VLEESVVSED AGQYFTESGL FSTGGLYETV LEAHREFRKL FDSIVNKDHK FFGSKFGDLD ITKLPEISHR AEFVEVIKDS NKAYIQITFV DRVTYFEKNF FTLEGRPRGE LTTMHAFPYI EFVLTPIEVA LAVAINQEPP GSVGATVNQG EIPADPKLYR EFIMRCGEAV |
| | | | | | EKNKRLITAD NYNKLKENLR YKPIFRVESQ RKCETQLSQG S | QREYQQELKK PMIERKIPEL KRDSFHRSSF |
| 38 | DOCK8 | 4 | Q8NF504 | Entry version 160 (18 Sep. 2019) Sequence version 3 (21 Aug. 2007) | MTHLNSLDVQ DDLDVVFTPK EEGVELDPHV WLIVNRKNQG GSRKDFHKTL ECSEPAAQAG SGKGPVTACD RLENLLQQVS ARRTNRQAEL DAVEIRPVPE LVKLLTLKFE ALYDVKERKK SDQFKGFLRA ARSAVFSVTY IEKVLQQGEI KESDGGKSKE SFCQRLGKYR SSFFNVSTLE GRSSVGERRT ALSLEENGVG SSFFKQEGDR ADYKRSSSLQ RLEISTAPEI PVKPFPENRT TREVYVPHTV RLNFVNKLAS MCGEDASNAM EFLQEVYTAV EEVKIKLPAK FYHISCQQKQ SWLPILLNER ALEKLPPNYS NPPIKWAEGH VSSVHTQDNH LESQVTFPIR LEHELKLSII VLFLHLVLDK IAGQTANFSQ NSLHNSKDLS ASYVHYVFRL GAPTALLDPR AAVSSKLLQA AGTHSAADEE DRNCSRMSYY PAAPRPASKK VVSTGMVRET LLVKSMAQHV RTRFSDRFMD SEIAALLVKP NISLAFFLYD FNLIRHYCSQ LISMRLEFLR LNLFFMNADT SQNSSSCSSF LTSEYRQQHF | LAQELGDFTD ECRTLQPSLP RDCVQTYIRE SPEICGFKKT PKQTFESETL PRHLNVLCDV FDLRSLQPDK AEDFEKQNEE FALYPSVDEE CPKEHLGNRI IEIEPLFASI ISENFHCDLN HTPSVAASSQ PSSDIYLVVK GDCAEPYTVI KIEKLKLQAE MPFAWAPISL REVTDVDSW LAQSRRLSER SNFKTSTLSV LSDEDLFKFL RRVKSIPGLL INCCLTPEML RPHKEILEFP YRNLLYVYPQ ARNITIKIQF PVIFGKSSGP TYHNKSPDFY LTVNHHLLFT GASVETLLGY LQTGSYCLPV MHSAEKVPLQ KGVFNIEVQA LEKFFTLCHS VLDQKISEMA CLNSSRLEPL LFQLSVQPMV FAFESVVAIA KDQHGRNCLL PEVQRDVPKS SYHTYGRTSA RVMSSSNPDL VKNIMSSKIA CSGSSDAPSS HFHEELALQM VFKYAWFFFE HNMDKRDSFR DITTIVNVVT QKENEQAEKM LLSLMDRGFV LSAKLSNLPT ILCSHEHYLN APTSPCPSIS QDQKIASMFD LTGLLFTELA |
| | | | | | AALDAEGEGI IHSLLSSHDL VKIAALYLPL LCDFTVADTR QEGAGAINQN NLKTSGIVLS NADTTRNLMI QSLIRKWIAD DLLFICVLCF VSTQVLQKSR LRGEGARGEM FPGLNENLRW NEKLDKTKAE LATEAHLIIL SALDCKDSLL LNCDQSTTYL IAKFGDLLFE HQVLHHCSSS TLYLLMRFSF MQVTMSLASL HLRRSLRTIL MTPFPTQVEE DTVKMREFQE RIAKSYQASP AEKHTKKKCY AALVAEYLSM SVSFQNISSN TLSPDEDGVC VGLLEQAAEL NEVYKLVIPI TLTHSKLQRA RMFGTYFRVG EQEFVYKEPA LEAFYGQCFG TPVDKTKLDP EPYFDEYEMK NLRRFMYTTP LHEQYRRNTV KTRISVIQKE IEDMKKKTLQ DAKMLQMVLQ PLEVAQVFLA HHNKLRLCFK EKNKRLITAD NYNKLKENLR YKPIFRVESQ RKCETQLSQG | SKVQRKAVSA DPRCVKPEVK VGIILDALPQ RYRTSGSDEE VALAIAGNNF SLPYKQYNML CFLWIMKNAD LPSTQLNRIL EYKGKQSSDK DVKARLEEAL MRRRAPGNDR KKEQTHWRQA LDQEALISGN DMQENIIQAS GGVLRVLVNS THCFATLRAL EEVEQCFDLC MDVTRSQACA GATSNFARVK VGRAPDFNEE AYSEEDTAMQ LLCNLNSILY DPEMLMDLMY DLRLTWLQNM TEAAMCLVHA LEDHSYLPVG VLEESVVSED AGQYFTESGL FSTGGLYETV LEAHREFRKL FDSIVNKDHK FFGSKFGDLD ITKLPEISHR AEFVEVIKDS NKAYIQITFV DRVTYFEKNF FTLEGRPRGE LTTMHAFPYI EFVLTPIEVA LAVAINQEPP GSVGATVNQG EIPADPKLYR EFIMRCGEAV QREYQQELKK PMIERKIPEL KRDSFHRSSF S |
| 39 | COMP | 1 | P49747 | Entry version 195 (18 Sep. 2019) Sequence version 2 (14 Oct. 2008) | MVPDTACVLL QGQSPLGSDL TNAALQDVRE FLKNTVMECD GLPSVRPLLH ACIQTESGAR NGSHCTDVNE RCINTSPGFR PTHQGVGLAF INECETGQHN RGSFQCGPCQ CQRRAQRFCP | LTLAALGASG GPQMLRELQE LLRQQVREIT ACGMQQSVRT CAPGFCFPGV CGPCPAGFTG CNAHPCFPRV CEACPPGYSG AKANKQVCTD CVPNSVCINT PGFVGDQASG DGSPSECHEH |
| | | | | | ADCVLERDGS GNGILCGRDT RCPERQCRKD EDVDRDGIGD VPNEKDNCPL EDKWGDACDN DTDQDGRGDA RNQADNCPRV DGIGDACDNC VDHDFVGDAC HQDSRDNCPT HDGQGDACDD RDNCRLVPNP GDVCQDDFDA PENAEVTLTD PEGDAQIDPN VQTMNSDPGL DFEGTFHVNT FGYQDSSSFY YWQANPFRAV KSSTGPGEQL ESQVRLLWKD SYRWFLQHRP EGPELVADSN RLGVFCFSQE CNDTIPEDYE | RSCVCAVGWA DLDGFPDEKL NCVTVPNSGQ ACDPDADGDG VRNPDQRNTD CRSQKNDDQK CDDDIDGDRI PNSDQKDSDG PQKSNPDQAD DSDQDQDGDG VPNSAQEDSD DDDNDGVPDS GQEDADRDGV DKVVDKIDVC FRAFQTVVLD WVVLNQGREI AVGYTAFNGV VTDDDYAGFI VVMWKQMEQT AEPGIQLKAV RNALWHTGDT PRNVGWKDKK QVGYIRVRFY VVLDTTMRGG NIIWANLRYR THQLRQA |
| 40 | COMP | 2 | P497472 | Entry version 195 (18 Sep. 2019) Sequence version 2 (14 Oct. 2008) | MVPDTACVLL QGQSPLGSDL TNAALQDVRE FLKNTVMECD GLPSVRPLLH ACIQTESGAR NGSHCTDVNE NSVCINTRGS VGDQASGCQR PSECHEHADC VCAVGWAGNG GFPDEKLRCP TVPNSGQEDV PDADGDGVPN PDQRNTDEDK QKNDDQKDTD DIDGDRIRNQ DQKDSDGDGI SNPDQADVDH QDQDGDGHQD SAQEDSDHDG NDGVPDSRDN DADRDGVGDV VDKIDVCPEN FQTVVLDPEG LNQGREIVQT YTAFNGVDFE DDYAGFIFGY WKQMEQTYWQ GIQLKAVKSS LWHTGDTESQ VGWKDKKSYR YIRVRFYEGP | LTLAALGASG GPQMLRELQE LLRQQVREIT ACGMQQSVRT CAPGFCFPGV CGPCPAGFTG CETGQHNCVP FQCGPCQPGF RAQRFCPDGS VLERDGSRSC ILCGRDTDLD ERQCRKDNCV DRDGIGDACD EKDNCPLVRN WGDACDNCRS QDGRGDACDD ADNCPRVPNS GDACDNCPQK DFVGDACDSD SRDNCPTVPN QGDACDDDDD CRLVPNPGQE CQDDFDADKV AEVTLTDFRA DAQIDPNWW MNSDPGLAVG GTFHVNTVTD QDSSSFYVVM ANPFRAVAEP TGPGEQLRNA VRLLWKDPRN WFLQHRPQVG ELVADSNVVL |
| | | | | | DTTMRGGRLG WANLRYRCND LRQA | VFCFSQENII TIPEDYETHQ |
| 41 | ATG7 | 1 | O95352 | Entry version 168 (18 Sep. 2019) Sequence version 1 (01 May 1999) | MAAATGDPGL ALDVGFWHEL DEAPKDIKGY ARLTLEFSAF CPAIGTLYNT KKLLLEQAAN ALENPVLLNK KYHFYYWFCY LIQGPVGLDQ ECAYDNLCQT IKYDENMVLV QGQRTKITIG PGWPLRNFLV QSVEVVCFRD HSIIFEVKLP AVGWEKNQKG ECMDPKRLAE CWRLVPTLDL LGAGTLGCNV HITFVDNAKI YEFEDCLGGG LQKIFPGVNA GHPVNFSSVT LEQLIESHDV RWLPAVIAAS GFDTFWMRH GDLCPNHPVA ANIPGYKLGC DSTRDRTLDQ VIAGALAVEL GYAIASSSDD LVPHQIRGFL LAFDKCTACS GFNFLAKVFN GLTLLHQETQ ETI | SKLQFAPFSS TQKKLNEYRL YYNGDSAGLP DMSAPTPARC NTLESFKTAD EIWESIKSGT FLLLTFADLK PALCLPESLP RFSLKQIEAL EGVTALPYFL SLLKHYSDFF VYDPCNLAQY LAAHRWSSSF RTMQGARDVA EMAFSPDCPK GMGPRMVNLS SSVDLNLKLM DKVVSVKCLL ARTLMGWGVR SYSNPVRQPL KPKALAAADR RGFNMSIPMP LEQARRDVEQ VFLLMDTRES KRKLVINAAL GLKKPKQQGA SADLLGSSLF YFCNDWAPG QCTVSRPGLA MVSVLQHPEG RMNEPPTSLG SRFDNVLPVS SKVLDQYERE SSHSFLEDLT AAEIWDMSDD |
| 42 | ATG7 | 2 | O953522 | Entry version 168 (18 Sep. 2019) Sequence version 1 (01 May 1999) | MAAATGDPGL ALDVGFWHEL DEAPKDIKGY ARLTLEFSAF CPAIGTLYNT KKLLLEQAAN ALENPVLLNK KYHFYYWFCY LIQGPVGLDQ ECAYDNLCQT IKYDENMVLV QGQRTKITIG PGWPLRNFLV QSVEVVCFRD HSIIFEVKLP AVGWEKNQKG ECMDPKRLAE CWRLVPTLDL LGAGTLGCNV | SKLQFAPFSS TQKKLNEYRL YYNGDSAGLP DMSAPTPARC NTLESFKTAD EIWESIKSGT FLLLTFADLK PALCLPESLP RFSLKQIEAL EGVTALPYFL SLLKHYSDFF VYDPCNLAQY LAAHRWSSSF RTMQGARDVA EMAFSPDCPK GMGPRMVNLS SSVDLNLKLM DKVVSVKCLL ARTLMGWGVR |
| | | | | | HITFVDNAKI YEFEDCLGGG LQKIFPGVNA GHPVNFSSVT LEQLIESHDV RWLPAVIAAS GFDTFWMRH GDLCPNHPVA ANIPGYKLGC DSTRDRTLDQ VIAGALAVEL GYAIASSSDD LVPHQVLDQY VFNSSHSFLE ETQAAEIWDM | SYSNPVRQPL KPKALAAADR RGFNMSIPMP LEQARRDVEQ VFLLMDTRES KRKLVINAAL GLKKPKQQGA SADLLGSSLF YFCNDWAPG QCTVSRPGLA MVSVLQHPEG RMNEPPTSLG EREGFNFLAK DLTGLTLLHQ SDDETI |
| 43 | ATG7 | 3 | O953523 | Entry version 168 (18 Sep. 2019) Sequence version 1 (01 May 1999) | MAAATGDPGL ALDVGFWHEL DEAPKDIKGY ARLTLEFSAF CPAIGTLYNT KKLLLEQAAN ALENPVLLNK LECAYDNLCQ LIKYDENMVL FQGQRTKITI YPGWPLRNFL FQSVEVVCFR AHSIIFEVKL KAVGWEKNQK SECMDPKRLA MCWRLVPTLD LLGAGTLGCN RHITFVDNAK LYEFEDCLGG RLQKIFPGVN TLEQARRDVE VVFLLMDTRE SRWLPAVIAA LGFDTFVVMR AGDLCPNHPV FANIPGYKLG GDSTRDRTLD AVIAGALAVE GGYAIASSSD GLVPHQIRGF SLAFDKCTAC ETI | SKLQFAPFSS TQKKLNEYRL YYNGDSAGLP DMSAPTPARC NTLESFKTAD EIWESIKSGT FLLLTFAIEA TEGVTALPYF VSLLKHYSDF GVYDPCNLAQ VLAAHRWSSS DRTMQGARDV PEMAFSPDCP GGMGPRMVNL ESSVDLNLKL LDKVVSVKCL VARTLMGWGV ISYSNPVRQP GKPKALAAAD ARGFNMSIPM PGHPVNFSSV QLEQLIESHD SKRKLVINAA HGLKKPKQQG ASADLLGSSL CYFCNDVVAP QQCTVSRPGL LMVSVLQHPE DRMNEPPTSL LSRFDNVLPV SSKIWDMSDD |
| 44 | SLC2A1 | 1 | P11166 | Entry version 230 (18 Sep. 2019) Sequence version 2 (03 Oct. 2006) | MEPSSKKLTG LGSLQFGYNT EEFYNQTWVH LTTLWSLSVA FSVGLFVNRF LLAFVSAVLM MLILGRFIIG PMYVGEVSPT QLGIVVGILI GNKDLWPLLL CIVLPFCPES | RLMLAVGGAV GVINAPQKVI RYGESILPTT IFSVGGMIGS GRRNSMLMMN GFSKLGKSFE VYCGLTTGFV ALRGALGTLH AQVFGLDSIM SIIFIPALLQ PRFLLINRNE |
| | | | | | ENRAKSVLKK LQEMKEESRQ ELFRSPAYRQ SQQLSGINAV AGVQQPVYAT TVVSLFVVER LAGMAGCAIL LPWMSYLSIV VGPGPIPWFI PAAIAVAGFS CFQYVEQLCG LVLFFIFTYF DEIASGFRQG ELFHPLGADS | LRGTADVTHD MMREKKVTIL PILIAVVLQL FYYSTSIFEK IGSGIVNTAF AGRRTLHLIG MTIALALLEQ AIFGFVAFFE VAELFSQGPR NWTSNFIVGM PYVFIIFTVL KVPETKGRTF GASQSDKTPE QV |
| 45 | GZMK | 1 | P49863 | Entry version 152 (18 Sep. 2019) Sequence version 1 (01 Oct. 1996) | MTKFSSFSLF VCFNMEIIGG MASIQYGGHH WVLTAAHCQY VLGAHSLSKN KFIPFSRVTS KLQTAAKLNK TSLRSGTKCK SLRPSDTLRE CNSQSYYNGD GDAKGQKDSC KGVFHAIVSG GIYTLLTKKY PHTN | FLIVGAYMTH KEVSPHSRPF VCGGVLIDPQ RFTKGQSPTV EASKQTLEIK DPQSNDIMLV HVKMLHIRSK VTGWGATDPD VTVTVLSRKL PFITKDMVCA KGDSGGPLIC GHECGVATKP QTWIKSNLVP |
| 46 | S100A9 | 1 | P06702 | Entry version 218 (18 Sep. 2019) Sequence version 1 (01 Jan. 1988) | MTCKMSQLER QYSVKLGHPD VRKDLQNFLK HIMEDLDTNA MLMARLTWAS PGHHHKPGLG | NIETIINTFH TLNQGEFKEL KENKNEKVIE DKQLSFEEFI HEKMHEGDEG EGTP |
| 47 | S100A8 | 1 | P05109 | Entry version 209 (18 Sep. 2019) Sequence version 1 (01 Jan. 1988) | MLTELEKALN LIKGNFHAVY ECPQYIRKKG NTDGAVNFQE AAHKKSHEES | SIIDVYHKYS RDDLKKLLET ADVWFKELDI FLILVIKMGV HKE |
| 48 | ATM | 1 | Q13315 | Entry version 236 (18 Sep. 2019) Sequence version 4 (22 Jan. 2014) | MSLVLNDLLI TERKKEVEKF KHLDRHSDSK FRFLQKYIQK NVSASTQASR VKYFIKCANR LNYIMDTVKD CSNILLKDIL QQQWLELFSV DVHRVLVARI QTDGLNSKFL RQEKSSSGLN KTLAVNFRIR TLLYIWTQHR ELFQLQIYIH GAYESTKWRS NEISHIGSRG VKENLIELMA TRSLEISQSY | CCRQLEHDRA KRLIRDPETI QGKYLNWDAV ETECLRIAKP QKKMQEISSL RAPRLKCQEL SSNGAIYGAD SVRKYWCEIS YFRLYLKPSQ IHAVTKGCCS DFFSKAIQCA HILAALTIFL VCELGDEILP LNDSLKEVII HPKGAKTQEK ILYNLYDLLV KYSSGFRNIA DICHQVFNED TTTQRESSDY |
| | | | | | SVPCKRKKIE QKSQNDFDLV SKYPASLPNC QLLPQQRHGE EVALCQDKRS LKLWNKIWCI QAENFGLLGA REFWKLFTGS CLTLALTTSI QNMCEVNRSF LFYQLEGDLE SNFPHLVLEK KAAMNFFQSV EELSFSEVEE DFLTIVRECG SVHQNLKESL LLNNYSSEIT LLVGVLGCYC YKSELFQKAK TLFKNKTNEE QLCTRCLSNC GFFLRLLTSK SLASFIKKPF DTNGNLMEVE YPDSSVSDAN AINPLAEEYL LKFLCLCVTT ADIRRKLLML SLHLHMYLML LPMEDVLELL RRDQDVCKTI LGQSNMDSEN VIGAFWHLTK ALVNCLKTLL LNVMGKDFPV NHHQVRMLAA KGDSSRLLKA ENAYLKAQEG PETLDEIYNR VLSCSPICEK KENGLEPHLV FGYRRLEDFM WLNLQDTEYN YTNIEDFYRS IRSHFDEVKS SLLTDCFPKI EGTRDSGMAQ MLKSENLLGK PEIVVELLMT QSTDLCDFSG FPSHVIKATF LKSILEILSK AICEQAAETN IYHLFVSLLL WAFVLRDVIY SCIMDVSLRS VCQTAVTYCK VGTLIPLVYE LLKYLVIDNK LLDPFPDHVV | LGWEVIKDHL PWLQIATQLI ELSPLLMILS RTPYVLRCLT NLESSQKSDL TFRGISSEQI IIQGSLVEVD ACRPSCPAVC VPGTVKMGIE SLKESIMKWL NSTEVPPILH ILVSLTMKNC PECEHHQKDK LFLQTTFDKM IEKHQSSIGF DRCLLGLSEQ NSETLVRCSR YMGVIAEEEA SLMQCAGESI FRIGSLRNMM TKKSPNKIAS LMNDIADICK DRGEVESMED DQSSMNLFND EPGESQSTIG SKQDLLFLDM AQTNTVSFRA IDSSTLEPTK LKELPGEEYP KPLSNVCSLY LNHVLHVVKN TRDAQGQFLT ERKYIFSVRM EADPYSKWAI NEVFTQFLAD ESINRLFQDT LPLKLQQTAF MREMSHSAEN KSVLLTLIAV QALFALCKSV KKVLEKVSET ASHLDYLVLE LSSFPFILLN CYKVLIPHLV IANQIQEDWK LVNILPYFAY QRETATKVYD QIDHLFISNL LHEPANSSAS DLDPAPNPPH AYISNCHKTK SPDSYQKILL NVYKKHRILK KDIKSGLGGA TLIHYINQRP FSLCCDLLSQ DALENHLHVI QVEVQKQVLD DNENLYITIK FKDLRITQQK |
| | | | | | IKYSRGPFSL SVYDALPLTR LELHKDQMVD DGIMVKLVVN HTGEKEVLEA IDFSTIAIQH KLFEDKELQW TLVEDCVKVR LATKTGHSFW LAYLQPFRTS DKENPFEGLD NHDIWIKTLT CEILQLLKPM VLPYLIHDIL LLSTHVQGFF SRSTTPANLD LDKKSQRTML RPSSGTIFND VAKVAQSCAA YADKKSMDDQ SQSTTISSLS LQDLLLEIYR CGGGKMLQPI MWGKALVTYD QAGIIQALQN LKGLDYENKD QAAWRNMQWD GTSYHESLYN STFYESLKYA RSLESVYSLY ELESIGELFS VYIKWQKHSQ EPIMALRTVI NSQRECIKDI LARTFKNTQL YNSVSCGVSE AKKEQSLALS ASCAANNPSL CGNWLAETCL LEKAVEVAGN NGKMKAFLSL IENYMKSSEF KEEVGLLREH VQRELELDEL RFLCKAVENY DMWVFRLCSL NGMMKRDGMK YQLAARMGTK LNNLISRISM LALANANRDE SRITKNVPKQ AANRIICTIR EALCDAYIIL QRKGINIPAD DVVVPTMEIK VTIQSFKAEF IIDCVGSDGK DLRQDAVMQQ RNTETRKRKL SQRSGVLEWC | LEEINHFLSV LEGLKDLRRQ IMRASQDNPQ LLQLSKMAIN VGSCLGEVGP SKDASYTKAL TFIMLTYLNN SAAVTCLKNI EIYKMTTDPM RKKFLEVPRF DINLWIPLSE CAFLDSGGTK CEVKTDFCQT LQDTNESWRN TSCLRHFSQT SESEHFFRCC AVVDYMRRQK AFWLDLNYLE HFTALLYAEI EKRSLAFEEG EKSKEETGIS SIGEPDSLYG TRLRTYEHEA LETAIPSSTR LGLCHILSVY WCPELEELHY HCTSVSKEVE ALQSLRDREF RVKEVEEMCK PTLSRLQAIG RSVTHRQLSE LLKDSDFSFQ LEILMEKEMD LTKHLVELSI PERAIFQIKQ WQLEEAQVFW ILKQMIKKLD KLTYTECLRV ENPAVIMQTY YDGESSDELR ARFSDTQYQR ENKQALLKRA KIQTNRYTVK ALRALKEDRK INCLLSGEEH WLENSGVSEV IPTYKFLPLM MMGGLGFHEV DHPHHTLFII FLTKPEVARR SSQLDEDRTE SRRPQMVRSV ANLDATQWKT QPITKLKNLE VDHTGEYGNL RLAGGVNLPK ERRQLVKGRD VFQMCNTLLQ TICTYKWPL TGTVPIGEFL |
| | | | | | VNNEDGAHKR CQKKMMEVQK MDVCQNFQPV DPAIWFEKRL IVGYILGLGD QSAELVHIDL PTPETVPFRL TGVEGVFRRC SQETLLTIVE TMNPLKALYL HPTLNADDQE SFNKVAERVL EEGTVLSVGG DPKNLSRLFP | YRPNDFSAFQ KSFEEKYEVF FRYFCMEKFL AYTRSVATSS RHVQNILINE GVAFEQGKIL TRDIVDGMGI CEKTMEVMRN VLLYDPLFDW QQRPEDETEL CKRNLSDIDQ MRLQEKLKGV QVNLLIQQAI GWKAWV |
| 49 | IKBKB | 1 | O14920 | Entry version 217 (18 Sep. 2019) Sequence version 1 (01 Jan. 1998) | MSWSPSLTTQ LGTGGFGNVI IAIKQCRQEL EIQIMRRLTH EGMQNLAPND GGDLRKYLNQ AILTLLSDIA IIHRDLKPEN IHKIIDLGYA SFVGTLQYLA VTVDYWSFGT PFLPNWQPVQ VDIWSEDLN YPNNLNSVLA LMWHPRQRGT KALDDILNLK TIHTYPVTED QQDTGIPEED LIPDKPATQC TLDMDLVFLF ISPRPQPESV LAFFQLRKVW KEDCNRLQQG NNSCLSKMKN AKLDFFKTSI TEFGITSDKL VELCGRENEV QTDIVDLQRS DDLEEQAREL QRTEGDSQEM FEKKVRVIYT KALELLPKVE KTVVRLQEKR ACSKVRGPVS LSQPGQLMSQ PAKKSEELVA NAIQDTVREQ WLQTEEEEHS | TCGAWEMKER RWHNQETGEQ SPRNRERWCL PNVVAARDVP LPLLAMEYCQ FENCCGLREG SALRYLHENR IVLQQGEQRL KELDQGSLCT PELLEQQKYT LAFECITGFR WHSKVRQKSE GTVKFSSSLP ERLEKWLQLM DPTYGPNGCF LVHILNMVTG ESLQSLKARI QELLQEAGLA ISDGKLNEGH DNSKITYETQ SCILQEPKRN GQVWHSIQTL QRAAMMNLLR SMASMSQQLK QIDLEKYSEQ LLAWREMEQA KLLVERMMAL PMGRKQGGTL YRRLREKPRD VRLLLQAIQS QLSKTWCKQ EVVSLMNEDE QKELWNLLKI GSPDSMNASR PSTASNSLPE EAHNLCTLLE DQSFTALDWS CLEQAS |
| 50 | IKBKB | 2 | O149202 | Entry version 217 (18 Sep. 2019) Sequence version 1 (01 Jan. 1998) | MFSGGCHSPG PGSPPPAPRP IKQCRQELSP QIMRRLTHPN MQNLAPNDLP DLRKYLNQFE LTLLSDIASA HRDLKPENIV | FGRPSPAFPA CRQETGEQIA RNRERWCLEI VVAARDVPEG LLAMEYCQGG NCCGLREGAI LRYLHENRII LQQGEQRLIH |
| | | | | | KIIDLGYAKE VGTLQYLAPE VDYWSFGTLA LPNWQPVQWH IVVSEDLNGT NNLNSVLAER WHPRQRGTDP LDDILNLKLV HTYPVTEDES DTGIPEEDQE PDKPATQCIS DMDLVFLFDN PRPQPESVSC FFQLRKVWGQ DCNRLQQGQR SCLSKMKNSM LDFFKTSIQI FGITSDKLLL LCGRENEVKL DIVDLQRSPM LEEQARELYR TEGDSQEMVR KKVRVIYTQL LELLPKVEEV VVRLQEKRQK SKVRGPVSGS QPGQLMSQPS KKSEELVAEA IQDTVREQDQ QTEEEEHSCL | LDQGSLCTSF LLEQQKYTVT FECITGFRPF SKVRQKSEVD VKFSSSLPYP LEKWLQLMLM TYGPNGCFKA HILNMVTGTI LQSLKARIQQ LLQEAGLALI DGKLNEGHTL SKITYETQIS ILQEPKRNLA VWHSIQTLKE AAMMNLLRNN ASMSQQLKAK DLEKYSEQTE AWREMEQAVE LVERMMALQT GRKQGGTLDD RLREKPRDQR LLLQAIQSFE SKTVVCKQKA VSLMNEDEKT ELWNLLKIAC PDSMNASRLS TASNSLPEPA HNLCTLLENA SFTALDWSWL EQAS |
| 51 | IKBKB | 3 | O149203 | Entry version 217 (18 Sep. 2019) Sequence version 1 (01 Jan. 1998) | MSWSPSLTTQ LGTGGFGNVI SPRNRERWCL EIQIMRRLTH EGMQNLAPND GGDLRKYLNQ AILTLLSDIA IIHRDLKPEN IHKIIDLGYA SFVGTLQYLA VTVDYWSFGT PFLPNWQPVQ HSCNPSTLGG | TCGAWEMKER RWHNQETGEQ IAIKQCRQEL PNVVAARDVP LPLLAMEYCQ FENCCGLREG SALRYLHENR IVLQQGEQRL KELDQGSLCT PELLEQQKYT LAFECITGFR CVRMWPGTVA RGRWIS |
| 52 | IKBKB | 4 | O149204 | Entry version 217 (18 Sep. 2019) Sequence version 1 (01 Jan. 1998) | MSSDGTIRLT PEGMQNLAPN QGGDLRKYLN GAILTLLSDI RIIHRDLKPE LIHKIIDLGY TSFVGTLQYL TVTVDYWSFG RPFLPNWQPV EVDIVVSEDL PYPNNLNSVL MLMWHPRQRG FKALDDILNL GTIHTYPVTE IQQDTGIPEE ALIPDKPATQ | HPNVVAARDV DLPLLAMEYC QFENCCGLRE ASALRYLHEN NIVLQQGEQR AKELDQGSLC APELLEQQKY TLAFECITGF QWHSKVRQKS NGTVKFSSSL AERLEKWLQL TDPTYGPNGC KLVHILNMVT DESLQSLKAR DQELLQEAGL CISDGKLNEG |
| | | | | | HTLDMDLVFL QISPRPQPES NLAFFQLRKV LKEDCNRLQQ RNNSCLSKMK KAKLDFFKTS QTEFGITSDK AVELCGRENE LQTDIVDLQR LDDLEEQARE DQRTEGDSQE SFEKKVRVIY QKALELLPKV EKTVVRLQEK IACSKVRGPV RLSQPGQLMS EPAKKSEELV ENAIQDTVRE SWLQTEEEEH | FDNSKITYET VSCILQEPKR WGQVWHSIQT GQRAAMMNLL NSMASMSQQL IQIDLEKYSE LLLAWREMEQ VKLLVERMMA SPMGRKQGGT LYRRLREKPR MVRLLLQAIQ TQLSKTVVCK EEWSLMNED RQKELWNLLK SGSPDSMNAS QPSTASNSLP AEAHNLCTLL QDQSFTALDW SCLEQAS |
| 53 | BCAP31 | 1 | P51572 | Entry version 186 (18 Sep. 2019) Sequence version 3 (23 Jan. 2007) | MSLQWTAVAT LLCIPFISPK VELLVSYGNT LLVIDAVREI NLQNNPGAME RNLYIAGFSL TLISQQATLL ESASEAAKKY AAVDGGKLDV NRSLKADLQK KLEKAENQVL KEYDRLLEEH MDKKEE | FLYAEVFVVL RWQKIFKSRL FFVVLIVILV RKYDDVTEKV HFHMKLFRAQ LLSFLLRRLV ASNEAFKKQA MEENDQLKKG GNAEVKLEEE LKDELASTKQ AMRKQSEGLT AKLQAAVDGP |
| 54 | BCAP31 | 2 | P515722 | Entry version 186 (18 Sep. 2019) Sequence version 3 (23 Jan. 2007) | MGAEASSSWC SVKRASEISG ALDVLTHVLE GKPSSNRMSL AEVFVVLLLC KIFKSRLVEL VLIVILVLLV DDVTEKVNLQ MKLFRAQRNL FLLRRLVTLI EAFKKQAESA NDQLKKGAAV EVKLEEENRS ELASTKQKLE KQSEGLTKEY QAAVDGPMDK | PGTALPEERL FLGQGSSGEA GAGNKLTSSC QWTAVATFLY IPFISPKRWQ LVSYGNTFFV IDAVREIRKY NNPGAMEHFH YIAGFSLLLS SQQATLLASN SEAAKKYMEE DGGKLDVGNA LKADLQKLKD KAENQVLAMR DRLLEEHAKL KEE |
| 55 | TAPBP | 1 | O15533 | Entry version 190 (18 Sep. 2019) Sequence version 1 (01 Jan. 1998) | MKSLSLLLAV GPAVIECWFV RPGALLLRQG DPELYLSVHD RYPRGAPAPH ASAKWASGLT GAWLMVSISS PQPEPQQEPV VLTHTPAPRV SFAYMPPTSE PFGLEWRRQH TPGLNGQMPA | ALGLATAVSA EDASGKGLAK PGEPPPRPDL PAGALQAAFR CEMSRFVPLP PAQNCPRALD PVLSLSSLLR LITMATVVLT RLGQDALLDL AASSLAPGPP LGKGHLLLAA AQEGAVAFAA |
| | | | | | WDDDEPWGPW VQPFQEGTYL QVTLELAVYK LARAAPGEAP YPSGGLEVEW QKAEGQRWLS SLSGHLQPPP ACRIHHPSLP EVAGLSGPSL FLLLGLFKAL KDSKKKAE | TGNGTFWLPR ATIHLPYLQG PPKVSLMPAT PELLCLVSHF ELRGGPGGRS ALRHHSDGSV VTTEQHGARY ASGRSAEVTL EDSVGLFLSA GWAAVYLSTC |
| 56 | TAPBP | 2 | O155332 | Entry version 190 (18 Sep. 2019) Sequence version 1 (01 Jan. 1998) | MKSLSLLLAV GPAVIECWFV RPGALLLRQG DPELYLSVHD RYPRGAPAPH ASAKWASGLT GAWLMVSISS PQPEPQQEPV VLTHTPAPRV SFAYMPPTSE PFGLEWRRQH TPGLNGQMPA WDDDEPWGPW VQPFQEGTYL QVTLELAVYK LARAAPGEAP YPSGGLEVEW QKAEGQRWLS SLSGHLQPPP ACRIHHPSLP EVAGKSWELC | ALGLATAVSA EDASGKGLAK PGEPPPRPDL PAGALQAAFR CEMSRFVPLP PAQNCPRALD PVLSLSSLLR LITMATVVLT RLGQDALLDL AASSLAPGPP LGKGHLLLAA AQEGAVAFAA TGNGTFWLPR ATIHLPYLQG PPKVSLMPAT PELLCLVSHF ELRGGPGGRS ALRHHSDGSV VTTEQHGARY ASGRSAEVTL GI |
| 58 | TAPBP | 4 | O15533 | Entry version | MKSLSLLLAV | ALGLATAVSA |
| | | | 4 | 190 (18 Sep. 2019) Sequence version 1 (01 Jan. 1998) | GPAVIECWFV RPGALLLRQG DPELYLSVHV PRVRLGQDAL TSEAASSLAP RQHLGKGHLL MPAAQEGAVA GPWTGNGTFW TYLATIHLPY VYKPPKVSLM EAPPELLCLV VEWELRGGPG WLSALRHHSD PPPVTTEQHG SLPASGRSAE PSLEDSVGLF KALGWAAVYL E | EDASGKGLAK PGEPPPRPDL VLTVLTHTPA LDLSFAYMPP GPPPFGLEWR LAATPGLNGQ FAAWDDDEPW LPRVQPFQEG LQGQVTLELA PATLARAAPG SHFYPSGGLE GRSQKAEGQR GSVSLSGHLQ ARYACRIHHP VTLEVAGLSG LSAFLLLGLF STCKDSKKKA |
| 59 | PPP3CB | 1 | P16298 | Entry version 166 (18 Sep. 2019) Sequence version 2 (01 Feb. 2005) | MAAPEPARAA PGADRVVKAV EEVFDLDGIP KEGRVDEEIA LRREKTMIEV HGQFFDLMKL RYLFLGDYVD YLWVLKILYP ECRHLTEYFT ERVYEACMEA NQQFLCVHGG IRRLDRFKEP WSDPSEDFGN TVRGCSYFYN NNLLSIIRAH RKSQTTGFPS LDVYNNKAAV RQFNCSPHPY WSLPFVGEKV CSDDELMTEG RKEIIRNKIR VLREESESVL LPSGVLAGGR IEAEKAIRGF EAKGLDRINE QDGFNSLNTA HTAQ | PPPPPPPPPP PFPPTHRLTS RVDVLKNHLV LRIINEGAAI EAPITVCGDI FEVGGSPANT RGYFSIECVL STLFLLRGNH FKQECKIKYS FDSLPLAALL LSPEIHTLDD PAFGPMCDLL EKSQEHFSHN YPAVCEFLQN EAQDAGYRMY LITIFSAPNY LKYENNVMNI WLPNFMDVFT TEMLVNVLSI EDQFDGSAAA AIGKMARVFS TLKGLTPTGM QTLQSATVEA SPPHRICSFE RMPPRKDAVQ HATENHGTGN |
| 60 | PPP3CB | 2 | P162982 | Entry version 166 (18 Sep. 2019) Sequence version 2 (01 Feb. 2005) | MAAPEPARAA PFPPTHRLTS EEVFDLDGIP KEGRVDEEIA LRREKTMIEV HGQFFDLMKL RYLFLGDYVD GTEDISINPH WVLKILYPST RHLTEYFTFK VYEACMEAFD QFLCVHGGLS RLDRFKEPPA DPSEDFGNEK | PPPPPPPPPP PGADRVVKAV RVDVLKNHLV LRIINEGAAI EAPITVCGDI FEVGGSPANT RGYFSIEHVL NNINECVLYL LFLLRGNHEC QECKIKYSER SLPLAALLNQ PEIHTLDDIR FGPMCDLLWS SQEHFSHNTV |
| | | | | | RGCSYFYNYP LLSIIRAHEA SQTTGFPSLI VYNNKAAVLK FNCSPHPYWL LPFVGEKVTE DDELMTEGED EIIRNKIRAI REESESVLTL SGVLAGGRQT AVPQMDWGTP CREFLLFFSS | AVCEFLQNNN QDAGYRMYRK TIFSAPNYLD YENNVMNIRQ PNFMDVFTWS MLVNVLSICS QFDGSAAARK GKMARVFSVL KGLTPTGMLP LQSGNDVMQL HSFANNSHNA CLSS |
| 61 | PPP3CB | 3 | P162983 | Entry version 166 (18 Sep. 2019) Sequence version 2 (01 Feb. 2005) | MAAPEPARAA PGADRVVKAV EEVFDLDGIP KEGRVDEEIA LRREKTMIEV HGQFFDLMKL RYLFLGDYVD YLWVLKILYP ECRHLTEYFT ERVYEACMEA NQQFLCVHGG IRRLDRFKEP WSDPSEDFGN TVRGCSYFYN NNLLSIIRAH RKSQTTGFPS LDVYNNKAAV RQFNCSPHPY WSLPFVGEKV CSDDELMTEG ARKEIIRNKI SVLREESESV MLPSGVLAGG FSPPHRICSF ERMPPRKDAV AHATENHGTG | PPPPPPPPPP PFPPTHRLTS RVDVLKNHLV LRIINEGAAI EAPITVCGDI FEVGGSPANT RGYFSIECVL STLFLLRGNH FKQECKIKYS FDSLPLAALL LSPEIHTLDD PAFGPMCDLL EKSQEHFSHN YPAVCEFLQN EAQDAGYRMY LITIFSAPNY LKYENNVMNI WLPNFMDVFT TEMLVNVLSI EDQFDVGSAA RAIGKMARVF LTLKGLTPTG RQTLQSAIRG EEAKGLDRIN QQDGFNSLNT NHTAQ |
| 62 | PPP3CB | 4 | P162984 | Entry version 166 (18 Sep. 2019) Sequence version 2 (01 Feb. 2005) | MAAPEPARAA PGADRVVKAV EEVFDLDGIP KEGRVDEEIA LRREKTMIEV HGQFFDLMKL RYLFLGDYVD YLWVLKILYP ECRHLTEYFT ERVYEACMEA NQQFLCVHGG IRRLDRFKEP WSDPSEDFGN TVRGCSYFYN NNLLSIIRAH RKSQTTGFPS LDVYNNKAAV RQFNCSPHPY WSLPFVGEKV CSDDELMTEG ARKEIIRNKI SVLREESESV | PPPPPPPPPP PFPPTHRLTS RVDVLKNHLV LRIINEGAAI EAPITVCGDI FEVGGSPANT RGYFSIECVL STLFLLRGNH FKQECKIKYS FDSLPLAALL LSPEIHTLDD PAFGPMCDLL EKSQEHFSHN YPAVCEFLQN EAQDAGYRMY LITIFSAPNY LKYENNVMNI WLPNFMDVFT TEMLVNVLSI EDQFDVGSAA RAIGKMARVF LTLKGLTPTG |
| | | | | | MLPSGVLAGG AIEAEKAIRG EEAKGLDRIN QQDGFNSLNT NHTAQ | RQTLQSATVE FSPPHRICSF ERMPPRKDAV AHATENHGTG |
| 63 | ANXA1 | 1 | P04083 | Entry version 244 (18 Sep. 2019) Sequence version 2 (23 Jan. 2007) | MAMVSEFLKQ YVQTVKSSKG TFNPSSDVAA DEATIIDILT KAAYLQETGK TCHLEEVVLA DELRAAMKGL LASRTNKEIR KRDLAKDITS LSLAKGDRSE SDARALYEAG FNTILTTRSY TKYSKHDMNK EKCLTAIVKC KLHQAMKGVG VSRSEIDMND ISLCQAILDE ALCGGN | AWFIENEEQE GPGSAVSPYP LHKAIMVKGV KRNNAQRQQI PLDETLKKAL LLKTPAQFDA GTDEDTLIEI DINRVYREEL DTSGDFRNAL DFGVNEDLAD ERRKGTDVNV PQLRRVFQKY VLDLELKGDI ATSKPAFFAE TRHKALIRIM IKAFYQKMYG TKGDYEKILV |
| 64 | PERM | 1 | P05164 | Entry version 221 (18 Sep. 2019) Sequence version 1 (13 Aug. 1987) | MGVPFFSSLR GGLTAEMKLL ATPQPSEGAA LVLSSMEEAK RESIKQRLRS YFKQPVAATR VALDLLERKL TDVLTPAQLN QDVGVTCPEQ NNRRSPTLGA AEYEDGFSLP GFPVALARAV QLTPDQERSL HDLDFTPEPA CETSCVQQPP PRIKNQADCI GSNITIRNQI MVYGSEEPLA GLLAVNQRFQ NLHDDPCLLT AGDTRSSEMP REHNRLATEL RLYQEARKIV DYLPLVLGPT SYNDSVDPRI GHTLIQPFMF RLDNRYQPME GGIDPILRGL NQIAVDEIRE LDLPALNMQR AWRRFCGLPQ LRNLKLARKL DIWMGGVSEP LACIIGTQFR ENEGVFSMQQ RIICDNTGIT | CMVDLGPCWA LALAGLLAIL PAVLGEVDTS QLVDKAYKER GSASPMELLS TAVRAADYLH RSLWRRPFNV VLSKSSGCAY DKYRTITGMC SNRAFVRWLP YGWTPGVKRN SNEIVRFPTD MFMQWGQLLD ARASFVTGVN CFPLKIPPND PFFRSCPACP NALTSFVDAS RNLRNMSNQL DNGRALLPFD NRSARIPCFL ELTSMHTLLL KSLNPRWDGE GAMVQIITYR AMRKYLPTYR ANVFTNAFRY PNPRVPLSRV FFASWRWLE MATPAKLNRQ RLFEQVMRIG SRDHGLPGYN PETVGQLGTV MEQYGTPNNI LKRKGRVGPL KLRDGDRFWW RQALAQISLP TVSKNNIFMS |
| | | | | | NSYPRDFVNC WREAS | STLPALNLAS |
| 65 | PERM | 2 | P051642 | Entry version 221 (18 Sep. 2019) Sequence version 1 (13 Aug. 1987) | MELLSYFKQP ADYLHVALDL RPFNVTDVLT SGCAYQDVGV ITGMCNNRRS VRWLPAEYED GVKRNGFPVA RFPTDQLTPD GQLLDHDLDF VTGVNCETSC IPPNDPRIKN CPACPGSNIT FVDASMVYGS MSNQLGLLAV LLPFDNLHDD IPCFLAGDTR HTLLLREHNR RWDGERLYQE IITYRDYLPL LPTYRSYNDS NAFRYGHTLI YQPMEPNPRV RWLEGGIDP KLNRQNQIAV VMRIGLDLPA LPGYNAWRRF QLGTVLRNLK TPNNIDIWMG RVGPLLACII DRFWWENEGV QISLPRIICD NIFMSNSYPR LNLASWREAS | VAATRTAVRA LERKLRSLWR PAQLNVLSKS TCPEQDKYRT PTLGASNRAF GFSLPYGWTP LARAVSNEIV QERSLMFMQW TPEPAARASF VQQPPCFPLK QADCIPFFRS IRNQINALTS EEPLARNLRN NQRFQDNGRA PCLLTNRSAR SSEMPELTSM LATELKSLNP ARKIVGAMVQ VLGPTAMRKY VDPRIANVFT QPFMFRLDNR PLSRVFFASW ILRGLMATPA DEIRERLFEQ LNMQRSRDHG CGLPQPETVG LARKLMEQYG GVSEPLKRKG GTQFRKLRDG FSMQQRQALA NTGITTVSKN DFVNCSTLPA |
| 66 | PERM | 3 | P051643 | Entry version 221 (18 Sep. 2019) Sequence version 1 (13 Aug. 1987) | MGVPFFSSLR GGLTAEMKLL ATPQPSEGAA LVLSSMEEAK RESIKQRLRS YFKQPVAATR VALDLLERKL TDVLTPAQLN QDVGVTCPEQ NNRCGWLGVA TPQASRCQRP GASNRAFVRW LPYGWTPGVK AVSNEIVRFP SLMFMQWGQL PAARASFVTG PPCFPLKIPP CIPFFRSCPA QINALTSFVD LARNLRNMSN FQDNGRALLP LTNRSARIPC MPELTSMHTL ELKSLNPRWD | CMVDLGPCWA LALAGLLAIL PAVLGEVDTS QLVDKAYKER GSASPMELLS TAVRAADYLH RSLWRRPFNV VLSKSSGCAY DKYRTITGMC AGTGLREASR VLPCRRSPTL LPAEYEDGFS RNGFPVALAR TDQLTPDQER LDHDLDFTPE VNCETSCVQQ NDPRIKNQAD CPGSNITIRN ASMVYGSEEP QLGLLAVNQR FDNLHDDPCL FLAGDTRSSE LLREHNRLAT GERLYQEARK |
| | | | | | IVGAMVQIIT PTAMRKYLPT RIANVFTNAF MFRLDNRYQP RVFFASWRVV GLMATPAKLN RERLFEQVMR QRSRDHGLPG PQPETVGQLG KLMEQYGTPN EPLKRKGRVG FRKLRDGDRF QQRQALAQIS ITTVSKNNIF NCSTLPALNL | YRDYLPLVLG YRSYNDSVDP RYGHTLIQPF MEPNPRVPLS LEGGIDPILR RQNQIAVDEI IGLDLPALNM YNAWRRFCGL TVLRNLKLAR NIDIWMGGVS PLLACIIGTQ WWENEGVFSM LPRIICDNTG MSNSYPRDFV ASWREAS |
| 67 | PLEC | 1 | Q15149 | Entry version 224 (18 Sep. 2019) Sequence version 3 (14 Oct. 2008) | MVAGMLMPRD REGVMVAKKD PGVTNLQVMR VRETFAWCHF HLRQYLHLPP RRPVAMVMPA GPLGSPPKRG RRKELEEVSP RTLARPGPEP QKKTFTKWVN SDLYEDLRDG SGDSLPREKG QIALDYLRHR DIADGNPKLT FQISDIQVSG LLLWSQRMVE TSSWRDGRLF LIDMNKVYRQ SVAERDLGVT PQPDEKSIIT RVPDVQDGVR YRELVLLLLQ ERRFPSSFEE FKEMELPAKE QSLEGAVQAG LDVEKEWGKL LRSEFERLEC EAGLCEEQLN RLLAAGKVPQ KADSMIRLLF HPQGEQMYRR IRTEYNLRLK AQVTLQSVQR YLQDLLAWVE WGVDLPSVEA SIEEFRAKIE PATRGAYRDC LLNSSKARLR AATKELMWLN WSDRNTNMTA ELELKEKKIK REDHPARPTV WSWMLQLCCC YFQFFSDVRE ALRRKYSCDR | QLRAIYEVLF RRPRSLHPHV AMASLRARGL YWYLTNEGIA EIVPASLQRV RRTPHVQAVQ PLPTEEQRVY ETPWPATTQ APATDERDRV KHLIKAQRHI HNLISLLEVL RMRFHKLQNV QVKLVNIRND LGLIWTIILH QSEDMTAKEK GYQGLRCDNF NAIIHRHKPL TNLENLDQAF RLLDPEDVDV YVSSLYDAMP ANELQLRWQE WMRHHTAAFE IEILWSQFLK ADKNRSKGIY QLKVPPGYHP HVAILEREKQ LQRIVTKLQM QADALLQSDV RAGEVERDLD NDVQTLKDGR VYRLHERLVA AGVAAPATQV RPELEDSTLR ENQHRVDGAE QLGSHRGLHQ RARSDEGQLS LGRLDLQYAK SLESLHSFVA EKEEEEVGFD KKESYSALMR ELQNAGDRLL ESFQAALQTQ IEAHLKENAA AEGQLQKLQE SATVTRLEDL |
| | | | | | LQDAQDEKEQ LAKRAKAVVQ RGRLPLLAVC KGDECQLVGP SSGSEAAVPS EAQEAVTRLE HQLHVDMKSL QLIRSWSLAT QALHSLELHY GGFGPEDRLM HYQQLLQSLE RCISELKDIR VHRLRLPLDK AEQQKAQAEV SAEAEKVLAL RSELELTLGK YLEKLKTISL VLRAHEEQLK ELEATKASLK PTFDALRDEL QQRHGERDVE LLERWQAVLA QLGRQLRYYR QDARRRQEQI VREQLRQEQA KVEECQRFAK LQLVTYKAQL KVQSGSESVI SELTTLTSQY MEEEERLAEQ EVEAALEKQR AQAEREAKEL REEAAVDAQQ QLRQSSEAEI IRVVRLQLEA TERQRGGAEG EAEAQKRQAQ EVELASRVKA ALQALEELRL QAEEAERRLR QVALETAQRS SFAEKTAQLE AQLREEAERR REEAERELER LRLQAEEVAQ KQKEEAEREA VRQRELAEQE TAQQRLAAEQ QGEQQRQLLE AAATQKRQEL MEVLLASKAR KSKQRLEAEA ARLRALAEEA AARQRAEAER EATRLKTEAE ERLRRLAEDE AAQHKADIEE SELERQKGLV EEEILALKAS LELELGRIRS | LNEYKGHLSG LKPRHPAHPM DYKQVEVTVH AQPSHWKVLS VCFLVPPPNQ AQHQALVTLW LAWQSLRRDV FRTLKPEEQR QAFLRDSQDA AEREYGSCSH QGAQEESRCQ LQLEACETRT EPARECAQRI EGLGKGVARL PEPSPAAPTL LEQVRSLSAI VIRGTQGAEE EAQAVPATLP KLRAQAEAQQ RGAQEVGERL VERWRERVAQ QTDVRQRELE ESADPLGAWL QAMPLADSQA LLEEIERHGE QYINAIKDYE EPVASPAKKP QEYVDLRTHY IKFISETLRR QRAEERERLA QLAEAHAQAK QQRMQEEWR QKRSIQEELQ QAKARQAEAA ERSRLRIEEE ELQALRARAE EEAERLRRQV QDESQRKRQA EAEAAREKQR QAEVERARQV AEAELQSKRA RSLQEEHVAV AQQQAEAERA WQLKANEALR QKSLAQAEAE RRRGKAEEQA LEKQRQLAEG ELIRLRAETE EELARLQREA EAELAKVRAE AEEESRSTSE GRFRELAEEA KRQRQLAEED VLAEKLAAIG IALKEKEAEN AFQRRRLEEQ RLAQLRKASD EDTLRQRRQV FEKAAAGKAE NAEDTLRSKE |
| | | | | | QAELEAARQR REAEERVQKS RKAALEEVER LRERAEQESA QKRLQAEEKA ELQQTLQQEQ EAARRAAEEA AAQSRRQVEE QAQARAQAQA QEAARRAQAE DAEMEKHKKF VEQELTTLRL LLDEELQRLK SQVEEELFSV KARIEAENRA RFLQEEAEKM SVAAQEAARL QRALAEKMLK RLKAEAELLQ RRLQEDKEQM FQRTLEAERQ RLKLRVAEMS QRFRKQAEEI TQEKVTLVQT DAERLREAIA QEAKLLQLKS LLQETQALQQ QRERFIEQEK VAKAQQLREE ERQRLVASME EGVRRKQEEL EELLAEENQR QHRAALAHSE KTLPNGRDAL HSFDGLRRKV LSAEELQRLA RREDVRHYLQ KATNEKLSVY GTALILLEAQ RNRRLTVNEA HHKLLSAERA QQISLFQAMQ RLLEAQIATG VPVDVAYRRG ADPSDDTKGF YLQLLERCVE LTDKAAKGGE VFEKATVSAP IWEIINSEYF QFRTGRITVE EEQEQKGRLC AELLESRVID ERSVRDVAEV NVIAGVWLEE LKKDLLPSDM TGHIIDPATS AGLVGPEFHE GYRDPYTGQS LIPREQGLRL VDPSKSHRVP | QLAAEEERRR LAAEEEAARQ LKAKVEEARR RQLQLAQEAA HAFAVQQKEQ SVLDQLRGEA EEARVQAERE AERLKQSAEE AAEKLRKEAE QAALRQKQAA AEQTLRQKAQ QLEETDHQKN AEATEAARQR RVQMEELSKL LILRDKDNTQ KQVAEEAARL RQLAEEDLAQ EKMQAVQEAT QQKELAQEQA AQQLAEETQG RQLEMSAEAE RAQARAEEDA GEKLHRTELA LEIQRQQSDH ELEREKEKLQ EEMQTVQQEQ SFLSEKDSLL AKLEQLFQDE QQRQQQQMEQ EARRRQHEAE QQLEQQRRQQ LREQLQLLEE EVTASQVAAT DGPAAEAEPE SAQRLQEAGI QGHTTVDELA GRSSIAGLLL AALQRQLLSP AASGFLLDPV VKEGVVGPEL VTGYKDPYTG KGLIVREHGI GVIDPVHSHR YFDEEMNRVL FDPNTHENLT DPETGLCLLP LVYTDSEARD FGKFQGKTVT TAEQRRDLLR KIIKIIITVV FEGLRSLVPA RELYQQLQRG DTVRRALRGA AGQKLSIYNA AVALLEAQAG ARLTVDEAVR KLLSAEKAVT VSLFQALKKG LDAQLSTGGI LDVACARGCL |
| | | | | | DEETSRALSA PSTGEPATYG LTGLSLLPLS LYSELQARET GGFKGRTVTV AEQRQELLRQ VIKILITIVE SFSGLRAPVP SRAQFEQLKD LGSVRTLLQG DTKEKVSIYE TAALLLEAQA NQRLYVHEAV EQLLSAEKAV TISLFQAMQK LLEAQIATGG PVDVAYQRGY DPSDDTKGFF RQLLERCVED KCAEKAEVVE RRAFEETQID TMSLWEVMQS LMADFQAGRV EIIEKTEIIR RRRLTAEDLF NLLREGTRSL CYLYGTGSVA LSIYQALKKG LEAQAATGFL VDEAVRKGLV AERAVTGYRD QAMKKELIPT LATGGIVDPR YQRGYLNKDT RSYVDPSTDE CRRDDGTGQL TFRGLRKQIT DEATALQLRE NLQKFLEGTS KERLSVYQAM FELLEAQAAT KLTVEEAVRM LLSAERAVTG SLFQAMKKGL EAQIATGGII EVAYKRGLFD SDDTKGFFDP LMERCITDPQ KKRERKTSSK VDPETGKEMS DHQTYLELSE SSSDGVVKSM DIDDAIAKNL AGTLSITEFA RSRSSSVGSS RTQLASWSDP LDTETLEKVS DNITGQRLLE PSTGERFPVT IMVDRINLAQ | PRADAKAYSD ELQQRCRPDQ EKAARARQEE FEKTPVEVPV WELISSEYFT FRTGKVTVEK EVETLRQERL ASELLASGVL GKTTVKDLSE SGCLAGIYLE AMRRGLLRAT ATGFLVDPVR KAGVVGPELH TGYRDPYSGS GLVLRQHGIR IIDPVHSHRV FSEEMNRVLA DPNTHENLTY PETGLRLLPL TTQVYTEEET IPGGGSHGGS DLIPEEQRAQ TKERMIIIII QQGLASYDYV EARIISLETY REALEAESAW GVYLPGSRQT LLSAEVARLL LDPVKGERLT GPELHDRLLS PYTEQTISLF EEALRLLDAQ LGFHLPLEVA HDQLSEPSEV RLSYTQLLRR LLPLSDARKL MEELVRSQVM GLTSIEEVTK CIAGVFVDAT KKGIIRPGTA GYVIDPIKGL GIVGPEFKDK YKDPYSGKLI ILKDHGIRLL DPEESHRLPV EEMNEILTDP NTEENLTYLQ TGLCLLPLKE SSVRKRRVVI VYEAYRKGLI QECEWEEITI IIDRRSGRQY IDRSALDQYR DMLSGNAGGF SSYPISPAVS TEETGPVAGI ITEAMHRNLV AQACTGGIID DAVNKGLVDK KAFCGFEDPR |
| | | | | | TKTKMSAAQA GQRFLEVQYL GRVPLDEALQ KLRDVGAYSK ISYKDALDRS LEAAAQSTKG GSTAGSRTGS GSFDATGSGF SSGYGRRYAS SAVA | LKKGWLYYEA TGGLIEPDTP RGTVDARTAQ YLTCPKTKLK MVEEGTGLRL YYSPYSVSGS RTGSRAGSRR SMTFSSSSYS GSSASLGGPE |
| 68 | PLEC | 2 | Q151492 | Entry version 224 (18 Sep. 2019) Sequence version 3 (14 Oct. 2008) | MSGEDAEVRA GSPSPGDTLP RSGGGAGSNG IRIADERDRV KHLIKAQRHI HNLISLLEVL RMRFHKLQNV QVKLVNIRND LGLIWTIILH QSEDMTAKEK GYQGLRCDNF NAIIHRHKPL TNLENLDQAF RLLDPEDVDV YVSSLYDAMP ANELQLRWQE WMRHHTAAFE IEILWSQFLK ADKNRSKGIY QLKVPPGYHP HVAILEREKQ LQRIVTKLQM QADALLQSDV RAGEVERDLD NDVQTLKDGR VYRLHERLVA AGVAAPATQV RPELEDSTLR ENQHRVDGAE QLGSHRGLHQ RARSDEGQLS LGRLDLQYAK SLESLHSFVA EKEEEEVGFD KKESYSALMR ELQNAGDRLL ESFQAALQTQ IEAHLKENAA AEGQLQKLQE SATVTRLEDL LNEYKGHLSG LKPRHPAHPM DYKQVEVTVH AQPSHWKVLS VCFLVPPPNQ AQHQALVTLW LAWQSLRRDV FRTLKPEEQR QAFLRDSQDA AEREYGSCSH | VSEDVSNGSS WNLGKTQRSR SVLDPAERAV QKKTFTKWVN SDLYEDLRDG SGDSLPREKG QIALDYLRHR DIADGNPKLT FQISDIQVSG LLLWSQRMVE TSSWRDGRLF LIDMNKVYRQ SVAERDLGVT PQPDEKSIIT RVPDVQDGVR YRELVLLLLQ ERRFPSSFEE FKEMELPAKE QSLEGAVQAG LDVEKEWGKL LRSEFERLEC EAGLCEEQLN RLLAAGKVPQ KADSMIRLLF HPQGEQMYRR IRTEYNLRLK AQVTLQSVQR YLQDLLAWVE WGVDLPSVEA SIEEFRAKIE PATRGAYRDC LLNSSKARLR AATKELMWLN WSDRNTNMTA ELELKEKKIK REDHPARPTV WSWMLQLCCC YFQFFSDVRE ALRRKYSCDR LQDAQDEKEQ LAKRAKAVVQ RGRLPLLAVC KGDECQLVGP SSGSEAAVPS EAQEAVTRLE HQLHVDMKSL QLIRSWSLAT QALHSLELHY GGFGPEDRLM HYQQLLQSLE |
| | | | | | QGAQEESRCQ LQLEACETRT EPARECAQRI EGLGKGVARL PEPSPAAPTL LEQVRSLSAI VIRGTQGAEE EAQAVPATLP KLRAQAEAQQ RGAQEVGERL VERWRERVAQ QTDVRQRELE ESADPLGAWL QAMPLADSQA LLEEIERHGE QYINAIKDYE EPVASPAKKP QEYVDLRTHY IKFISETLRR QRAEERERLA QLAEAHAQAK QQRMQEEWR QKRSIQEELQ QAKARQAEAA IRVVRLQLEA ELQALRARAE EEAERLRRQV EVELASRVKA ALQALEELRL QAEVERARQV AEAELQSKRA RSLQEEHVAV AQQQAEAERA WQLKANEALR QKSLAQAEAE RRRGKAEEQA LEKQRQLAEG ELIRLRAETE EELARLQREA EAELAKVRAE AEEESRSTSE GRFRELAEEA KRQRQLAEED VLAEKLAAIG IALKEKEAEN AFQRRRLEEQ RLAQLRKASD EDTLRQRRQV FEKAAAGKAE NAEDTLRSKE QLAAEEERRR LAAEEEAARQ LKAKVEEARR RQLQLAQEAA HAFAVQQKEQ SVLDQLRGEA EEARVQAERE AERLKQSAEE AAEKLRKEAE QAALRQKQAA | RCISELKDIR VHRLRLPLDK AEQQKAQAEV SAEAEKVLAL RSELELTLGK YLEKLKTISL VLRAHEEQLK ELEATKASLK PTFDALRDEL QQRHGERDVE LLERWQAVLA QLGRQLRYYR QDARRRQEQI VREQLRQEQA KVEECQRFAK LQLVTYKAQL KVQSGSESVI SELTTLTSQY MEEEERLAEQ EVEAALEKQR AQAEREAKEL REEAAVDAQQ QLRQSSEAEI ERSRLRIEEE TERQRGGAEG EAEAQKRQAQ QDESQRKRQA EAEAAREKQR QAEEAERRLR QVALETAQRS SFAEKTAQLE AQLREEAERR REEAERELER LRLQAEEVAQ KQKEEAEREA VRQRELAEQE TAQQRLAAEQ QGEQQRQLLE AAATQKRQEL MEVLLASKAR KSKQRLEAEA ARLRALAEEA AARQRAEAER EATRLKTEAE ERLRRLAEDE AAQHKADIEE SELERQKGLV EEEILALKAS LELELGRIRS QAELEAARQR REAEERVQKS RKAALEEVER LRERAEQESA QKRLQAEEKA ELQQTLQQEQ EAARRAAEEA AAQSRRQVEE QAQARAQAQA QEAARRAQAE DAEMEKHKKF |
| | | | | | AEQTLRQKAQ QLEETDHQKN AEATEAARQR RVQMEELSKL LILRDKDNTQ KQVAEEAARL RQLAEEDLAQ EKMQAVQEAT QQKELAQEQA AQQLAEETQG RQLEMSAEAE RAQARAEEDA GEKLHRTELA LEIQRQQSDH ELEREKEKLQ EEMQTVQQEQ SFLSEKDSLL AKLEQLFQDE QQRQQQQMEQ EARRRQHEAE QQLEQQRRQQ LREQLQLLEE EVTASQVAAT DGPAAEAEPE SAQRLQEAGI QGHTTVDELA GRSSIAGLLL AALQRQLLSP AASGFLLDPV VKEGVVGPEL VTGYKDPYTG KGLIVREHGI GVIDPVHSHR YFDEEMNRVL FDPNTHENLT DPETGLCLLP LVYTDSEARD FGKFQGKTVT TAEQRRDLLR KIIKIIITVV FEGLRSLVPA RELYQQLQRG DTVRRALRGA AGQKLSIYNA AVALLEAQAG ARLTVDEAVR KLLSAEKAVT VSLFQALKKG LDAQLSTGGI LDVACARGCL PRADAKAYSD ELQQRCRPDQ EKAARARQEE FEKTPVEVPV WELISSEYFT FRTGKVTVEK EVETLRQERL ASELLASGVL GKTTVKDLSE SGCLAGIYLE | VEQELTTLRL LLDEELQRLK SQVEEELFSV KARIEAENRA RFLQEEAEKM SVAAQEAARL QRALAEKMLK RLKAEAELLQ RRLQEDKEQM FQRTLEAERQ RLKLRVAEMS QRFRKQAEEI TQEKVTLVQT DAERLREAIA QEAKLLQLKS LLQETQALQQ QRERFIEQEK VAKAQQLREE ERQRLVASME EGVRRKQEEL EELLAEENQR QHRAALAHSE KTLPNGRDAL HSFDGLRRKV LSAEELQRLA RREDVRHYLQ KATNEKLSVY GTALILLEAQ RNRRLTVNEA HHKLLSAERA QQISLFQAMQ RLLEAQIATG VPVDVAYRRG ADPSDDTKGF YLQLLERCVE LTDKAAKGGE VFEKATVSAP IWEIINSEYF QFRTGRITVE EEQEQKGRLC AELLESRVID ERSVRDVAEV NVIAGVWLEE LKKDLLPSDM TGHIIDPATS AGLVGPEFHE GYRDPYTGQS LIPREQGLRL VDPSKSHRVP DEETSRALSA PSTGEPATYG LTGLSLLPLS LYSELQARET GGFKGRTVTV AEQRQELLRQ VIKILITIVE SFSGLRAPVP SRAQFEQLKD LGSVRTLLQG DTKEKVSIYE |
| | | | | | AMRRGLLRAT ATGFLVDPVR KAGVVGPELH TGYRDPYSGS GLVLRQHGIR IIDPVHSHRV FSEEMNRVLA DPNTHENLTY PETGLRLLPL TTQVYTEEET IPGGGSHGGS DLIPEEQRAQ TKERMIIIII QQGLASYDYV EARIISLETY REALEAESAW GVYLPGSRQT LLSAEVARLL LDPVKGERLT GPELHDRLLS PYTEQTISLF EEALRLLDAQ LGFHLPLEVA HDQLSEPSEV RLSYTQLLRR LLPLSDARKL MEELVRSQVM GLTSIEEVTK CIAGVFVDAT KKGIIRPGTA GYVIDPIKGL GIVGPEFKDK YKDPYSGKLI ILKDHGIRLL DPEESHRLPV EEMNEILTDP NTEENLTYLQ TGLCLLPLKE SSVRKRRVVI VYEAYRKGLI QECEWEEITI IIDRRSGRQY IDRSALDQYR DMLSGNAGGF SSYPISPAVS TEETGPVAGI ITEAMHRNLV AQACTGGIID DAVNKGLVDK KAFCGFEDPR LKKGWLYYEA TGGLIEPDTP RGTVDARTAQ YLTCPKTKLK MVEEGTGLRL YYSPYSVSGS RTGSRAGSRR SMTFSSSSYS GSSASLGGPE | TAALLLEAQA NQRLYVHEAV EQLLSAEKAV TISLFQAMQK LLEAQIATGG PVDVAYQRGY DPSDDTKGFF RQLLERCVED KGAEKAEVVE RRAFEETQID TMSLWEVMQS LMADFQAGRV EIIEKTEIIR RRRLTAEDLF NLLREGTRSL CYLYGTGSVA LSIYQALKKG LEAQAATGFL VDEAVRKGLV AERAVTGYRD QAMKKELIPT LATGGIVDPR YQRGYLNKDT RSYVDPSTDE CRRDDGTGQL TFRGLRKQIT DEATALQLRE NLQKFLEGTS KERLSVYQAM FELLEAQAAT KLTVEEAVRM LLSAERAVTG SLFQAMKKGL EAQIATGGII EVAYKRGLFD SDDTKGFFDP LMERCITDPQ KKRERKTSSK VDPETGKEMS DHQTYLELSE SSSDGVVKSM DIDDAIAKNL AGTLSITEFA RSRSSSVGSS RTQLASWSDP LDTETLEKVS DNITGQRLLE PSTGERFPVT IMVDRINLAQ TKTKMSAAQA GQRFLEVQYL GRVPLDEALQ KLRDVGAYSK ISYKDALDRS LEAAAQSTKG GSTAGSRTGS GSFDATGSGF SSGYGRRYAS SAVA |
| 69 | PLEC | 3 | Q15149-3 | Entry version | MSGEDAEVRA | VSEDVSNGSS |
| | | | | 224 (18 Sep. 2019) Sequence version 3 (14 Oct. 2008) | GSPSPGDTLP RSGGGAGSNG IRIADERDRV KHLIKAQRHI HNLISLLEVL RMRFHKLQNV QVKLVNIRND LGLIWTIILH QSEDMTAKEK GYQGLRCDNF NAIIHRHKPL TNLENLDQAF RLLDPEDVDV YVSSLYDAMP QLRWQEYREL HTAAFEERRF WSQFLKFKEM RSKGIYQSLE PPGYHPLDVE LEREKQLRSE VTKLQMEAGL LLQSDVRLLA VERDLDKADS TLKDGRHPQG HERLVAIRTE APATQVAQVT EDSTLRYLQD RVDGAEWGVD HRGLHQSIEE DEGQLSPATR DLQYAKLLNS LHSFVAAATK EEVGFDWSDR YSALMRELEL AGDRLLREDH AALQTQWSWM LKENAAYFQF LQKLQEALRR TRLEDLLQDA KGHLSGLAKR HPAHPMRGRL VEVTVHKGDE HWKVLSSSGS VPPPNQEAQE ALVTLWHQLH SLRRDVQLIR KPEEQRQALH RDSQDAGGFG YGSCSHHYQQ EESRCQRCIS ACETRTVHRL ECAQRIAEQQ KGVARLSAEA PAAPTLRSEL RSLSAIYLEK TQGAEEVLRA VPATLPELEA QAEAQQPTFD EVGERLQQRH RERVAQLLER | WNLGKTQRSR SVLDPAERAV QKKTFTKWVN SDLYEDLRDG SGDSLPREKG QIALDYLRHR DIADGNPKLT FQISDIQVSG LLLWSQRMVE TSSWRDGRLF LIDMNKVYRQ SVAERDLGVT PQPDEKSIIT RVPDVQDGEL VLLLLQWMRH PSSFEEIEIL ELPAKEADKN GAVQAGQLKV KEWGKLHVAI FERLECLQRI CEEQLNQADA AGKVPQRAGE MIRLLFNDVQ EQMYRRVYRL YNLRLKAGVA LQSVQRRPEL LLAWVEENQH LPSVEAQLGS FRAKIERARS GAYRDCLGRL SKARLRSLES ELMWLNEKEE NTNMTAKKES KEKKIKELQN PARPTVESFQ LQLCCCIEAH FSDVREAEGQ KYSCDRSATV QDEKEQLNEY AKAVVQLKPR PLLAVCDYKQ CQLVGPAQPS EAAVPSVCFL AVTRLEAQHQ VDMKSLLAWQ SWSLATFRTL SLELHYQAFL PEDRLMAERE LLQSLEQGAQ ELKDIRLQLE RLPLDKEPAR KAQAEVEGLG EKVLALPEPS ELTLGKLEQV LKTISLVIRG HEEQLKEAQA TKASLKKLRA ALRDELRGAQ GERDVEVERW WQAVLAQTDV |
| | | | | | RQRELEQLGR PLGAWLQDAR LADSQAVREQ IERHGEKVEE AIKDYELQLV SPAKKPKVQS DLRTHYSELT SETLRRMEEE ERERLAEVEA AHAQAKAQAE QEEWRREEA IQEELQQLRQ RQAEAAERSR RLQLEATERQ LRARAEEAEA RLRRQVQDES ASRVKAEAEA LEELRLQAEE ERARQVQVAL LQSKRASFAE EEHVAVAQLR AEAERAREEA ANEALRLRLQ AQAEAEKQKE KAEEQAVRQR RQLAEGTAQQ LRAETEQGEQ RLQREAAAAT AKVRAEMEVL SRSTSEKSKQ ELAEEAARLR QLAEEDAARQ KLAAIGEATR EKEAENERLR RRLEEQAAQH LRKASDSELE RQRRQVEEEI AAGKAELELE TLRSKEQAEL EEERRRREAE EEAARQRKAA VEEARRLRER LAQEAAQKRL VQQKEQELQQ QLRGEAEAAR VQAEREAAQS KQSAEEQAQA LRKEAEQEAA RQKQAADAEM LRQKAQVEQE TDHQKNLLDE EAARQRSQVE EELSKLKARI DKDNTQRFLQ EEAARLSVAA EEDLAQQRAL AVQEATRLKA LAQEQARRLQ AEETQGFQRT MSAEAERLKL | QLRYYRESAD RRQEQIQAMP LRQEQALLEE CQRFAKQYIN TYKAQLEPVA GSESVIQEYV TLTSQYIKFI ERLAEQQRAE ALEKQRQLAE REAKELQQRM AVDAQQQKRS SSEAEIQAKA LRIEEEIRVV RGGAEGELQA QKRQAQEEAE QRKRQAEVEL AREKQRALQA AERRLRQAEV ETAQRSAEAE KTAQLERSLQ EEAERRAQQQ ERELERWQLK AEEVAQQKSL EAEREARRRG ELAEQELEKQ RLAAEQELIR QRQLLEEELA QKRQELEAEL LASKARAEEE RLEAEAGRFR ALAEEAKRQR RAEAERVLAE LKTEAEIALK RLAEDEAFQR KADIEERLAQ RQKGLVEDTL LALKASFEKA LGRIRSNAED EAARQRQLAA ERVQKSLAAE LEEVERLKAK AEQESARQLQ QAEEKAHAFA TLQQEQSVLD RAAEEAEEAR RRQVEEAERL RAQAQAAAEK RRAQAEQAAL EKHKKFAEQT LTTLRLQLEE ELQRLKAEAT EELFSVRVQM EAENRALILR EEAEKMKQVA QEAARLRQLA AEKMLKEKMQ EAELLQQQKE EDKEQMAQQL LEAERQRQLE RVAEMSRAQA |
| | | | | | RAEEDAQRFR HRTELATQEK RQQSDHDAER EKEKLQQEAK TVQQEQLLQE EKDSLLQRER QLFQDEVAKA QQQMEQERQR RQHEAEEGVR QQRRQQEELL LQLLEEQHRA SQVAATKTLP AEAEPEHSFD LQEAGILSAE TVDELARRED IAGLLLKATN RQLLSPGTAL FLLDPVRNRR VVGPELHHKL KDPYTGQQIS VREHGIRLLE PVHSHRVPVD EMNRVLADPS THENLTYLQL GLCLLPLTDK DSEARDVFEK QGKTVTIWEI RRDLLRQFRT IIITVVEEQE RSLVPAAELL QQLQRGERSV RALRGANVIA LSIYNALKKD LEAQAGTGHI VDEAVRAGLV AEKAVTGYRD QALKKGLIPR LSTGGIVDPS CARGCLDEET AKAYSDPSTG RCRPDQLTGL RARQEELYSE PVEVPVGGFK SSEYFTAEQR KVTVEKVIKI LRQERLSFSG LASGVLSRAQ VKDLSELGSV AGIYLEDTKE GLLRATTAAL LVDPVRNQRL VGPELHEQLL DPYSGSTISL RQHGIRLLEA VHSHRVPVDV MNRVLADPSD HENLTYRQLL LRLLPLKGAE YTEEETRRAF GSHGGSTMSL | KQAEEIGEKL VTLVQTLEIQ LREAIAELER LLQLKSEEMQ TQALQQSFLS FIEQEKAKLE QQLREEQQRQ LVASMEEARR RKQEELQQLE AEENQRLREQ ALAHSEEVTA NGRDALDGPA GLRRKVSAQR ELQRLAQGHT VRHYLQGRSS EKLSVYAALQ ILLEAQAASG LTVNEAVKEG LSAERAVTGY LFQAMQKGLI AQIATGGVID VAYRRGYFDE DDTKGFFDPN LERCVEDPET AAKGGELVYT ATVSAPFGKF INSEYFTAEQ GRITVEKIIK QKGRLCFEGL ESRVIDRELY RDVAEVDTVR GVWLEEAGQK LLPSDMAVAL IDPATSARLT GPEFHEKLLS PYTGQSVSLF EQGLRLLDAQ KSHRVPLDVA SRALSAPRAD EPATYGELQQ SLLPLSEKAA LQARETFEKT GRTVTVWELI QELLRQFRTG LITIVEEVET LRAPVPASEL FEQLKDGKTT RTLLQGSGCL KVSIYEAMRR LLEAQAATGF YVHEAVKAGV SAEKAVTGYR FQAMQKGLVL QIATGGIIDP AYQRGYFSEE DTKGFFDPNT ERCVEDPETG KAEVVETTQV EETQIDIPGG WEVMQSDLIP |
| | | | | | EEQRAQLMAD MIIIIIEIIE ASYDYVRRRL ISLETYNLLR EAESAWCYLY PGSRQTLSIY EVARLLLEAQ KGERLTVDEA HDRLLSAERA QTISLFQAMK RLLDAQLATG LPLEVAYQRG SEPSEVRSYV TQLLRRCRRD SDARKLTFRG VRSQVMDEAT IEEVTKNLQK VFVDATKERL IRPGTAFELL DPIKGLKLTV PEFKDKLLSA YSGKLISLFQ HGIRLLEAQI SHRLPVEVAY EILTDPSDDT NLTYLQLMER LLPLKEKKRE KRRVVIVDPE YRKGLIDHQT WEEITISSSD RSGRQYDIDD ALDQYRAGTL GNAGGFRSRS ISPAVSRTQL GPVAGILDTE MHRNLVDNIT TGGIIDPSTG KGLVDKIMVD GFEDPRTKTK WLYYEAGQRF IEPDTPGRVP DARTAQKLRD PKTKLKISYK GTGLRLLEAA YSVSGSGSTA RAGSRRGSFD SSSSYSSSGY SLGGPESAVA | FQAGRVTKER KTEIIRQQGL TAEDLFEARI EGTRSLREAL GTGSVAGVYL QALKKGLLSA AATGFLLDPV VRKGLVGPEL VTGYRDPYTE KELIPTEEAL GIVDPRLGFH YLNKDTHDQL DPSTDERLSY DGTGQLLLPL LRKQITMEEL ALQLREGLTS FLEGTSCIAG SVYQAMKKGI EAQAATGYVI EEAVRMGIVG ERAVTGYKDP AMKKGLILKD ATGGIIDPEE KRGLFDEEMN KGFFDPNTEE CITDPQTGLC RKTSSKSSVR TGKEMSVYEA YLELSEQECE GWKSMIIDR AIAKNLIDRS SITEFADMLS SSVGSSSSYP ASWSDPTEET TLEKVSITEA GQRLLEAQAC ERFPVTDAVN RINLAQKAFC MSAAQALKKG LEVQYLTGGL LDEALQRGTV VGAYSKYLTC DALDRSMVEE AQSTKGYYSP GSRTGSRTGS ATGSGFSMTF GRRYASGSSA |
| 70 | PLEC | 4 | Q151494 | Entry version224 (18 Sep. 2019)Sequence version 3 (14 Oct. 2008) | MSQHQLRVPQ SEDNLYLAVL DRVQKKTFTK RHISDLYEDL EVLSGDSLPR QNVQIALDYL RNDDIADGNP ILHFQISDIQ KEKLLLWSQR DNFTSSWRDG KPLLIDMNKV | PEGLGRKRTS RASEGKKDER WVNKHLIKAQ RDGHNLISLL EKGRMRFHKL RHRQVKLVNI KLTLGLIWTI VSGQSEDMTA MVEGYQGLRC RLFNAIIHRH YRQTNLENLD |
| | | | | | QAFSVAERDL VDVPQPDEKS AMPRVPDVQD WQEYRELVLL AFEERRFPSS FLKFKEMELP GIYQSLEGAV YHPLDVEKEW EKQLRSEFER LQMEAGLCEE SDVRLLAAGK DLDKADSMIR DGRHPQGEQM LVAIRTEYNL TQVAQVTLQS TLRYLQDLLA GAEWGVDLPS LHQSIEEFRA QLSPATRGAY YAKLLNSSKA FVAAATKELM GFDWSDRNTN LMRELELKEK RLLREDHPAR QTQWSWMLQL NAAYFQFFSD LQEALRRKYS EDLLQDAQDE LSGLAKRAKA HPMRGRLPLL TVHKGDECQL VLSSSGSEAA PNQEAQEAVT TLWHQLHVDM RDVQLIRSWS EQRQALHSLE QDAGGFGPED CSHHYQQLLQ RCQRCISELK TRTVHRLRLP QRIAEQQKAQ ARLSAEAEKV PTLRSELELT SAIYLEKLKT AEEVLRAHEE TLPELEATKA AQQPTFDALR ERLQQRHGER VAQLLERWQA ELEQLGRQLR AWLQDARRRQ SQAVREQLRQ HGEKVEECQR DYELQLVTYK KKPKVQSGSE THYSELTTLT LRRMEEEERL RLAEVEAALE QAKAQAEREA VVRREEAAVD | GVTRLLDPED IITYVSSLYD GVRANELQLR LLQWMRHHTA FEEIEILWSQ AKEADKNRSK QAGQLKVPPG GKLHVAILER LECLQRIVTK QLNQADALLQ VPQRAGEVER LLFNDVQTLK YRRVYRLHER RLKAGVAAPA VQRRPELEDS WVEENQHRVD VEAQLGSHRG KIERARSDEG RDCLGRLDLQ RLRSLESLHS WLNEKEEEEV MTAKKESYSA KIKELQNAGD PTVESFQAAL CCCIEAHLKE VREAEGQLQK CDRSATVTRL KEQLNEYKGH VVQLKPRHPA AVCDYKQVEV VGPAQPSHWK VPSVCFLVPP RLEAQHQALV KSLLAWQSLR LATFRTLKPE LHYQAFLRDS RLMAEREYGS SLEQGAQEES DIRLQLEACE LDKEPARECA AEVEGLGKGV LALPEPSPAA LGKLEQVRSL ISLVIRGTQG QLKEAQAVPA SLKKLRAQAE DELRGAQEVG DVEVERWRER VLAQTDVRQR YYRESADPLG EQIQAMPLAD EQALLEEIER FAKQYINAIK AQLEPVASPA SVIQEYVDLR SQYIKFISET AEQQRAEERE KQRQLAEAHA KELQQRMQEE AQQQKRSIQE |
| | | | | | ELQQLRQSSE EAAERSRLRI LEATERQRGG RAEEAEAQKR RQVQDESQRK VKAEAEAARE LRLQAEEAER RQVQVALETA KRASFAEKTA VAVAQLREEA ERAREEAERE ALRLRLQAEE EAEKQKEEAE EQAVRQRELA AEGTAQQRLA ETEQGEQQRQ REAAAATQKR RAEMEVLLAS TSEKSKQRLE EEAARLRALA EEDAARQRAE AIGEATRLKT AENERLRRLA EEQAAQHKAD ASDSELERQK RQVEEEILAL KAELELELGR SKEQAELEAA RRRREAEERV ARQRKAALEE ARRLRERAEQ EAAQKRLQAE KEQELQQTLQ GEAEAARRAA EREAAQSRRQ AEEQAQARAQ EAEQEAARRA QAADAEMEKH KAQVEQELTT QKNLLDEELQ RQRSQVEEEL SKLKARIEAE NTQRFLQEEA ARLSVAAQEA LAQQRALAEK EATRLKAEAE EQARRLQEDK TQGFQRTLEA EAERLKLRVA EDAQRFRKQA ELATQEKVTL SDHDAERLRE KLQQEAKLLQ QEQLLQETQA SLLQRERFIE QDEVAKAQQL MEQERQRLVA EAEEGVRRKQ RQQEELLAEE LEEQHRAALA | AEIQAKARQA EEEIRVVRLQ AEGELQALRA QAQEEAERLR RQAEVELASR KQRALQALEE RLRQAEVERA QRSAEAELQS QLERSLQEEH ERRAQQQAEA LERWQLKANE VAQQKSLAQA REARRRGKAE EQELEKQRQL AEQELIRLRA LLEEELARLQ QELEAELAKV KARAEEESRS AEAGRFRELA EEAKRQRQLA AERVLAEKLA EAEIALKEKE EDEAFQRRRL IEERLAQLRK GLVEDTLRQR KASFEKAAAG IRSNAEDTLR RQRQLAAEEE QKSLAAEEEA VERLKAKVEE ESARQLQLAQ EKAHAFAVQQ QEQSVLDQLR EEAEEARVQA VEEAERLKQS AQAAAEKLRK QAEQAALRQK KKFAEQTLRQ LRLQLEETDH RLKAEATEAA FSVRVQMEEL NRALILRDKD EKMKQVAEEA ARLRQLAEED MLKEKMQAVQ LLQQQKELAQ EQMAQQLAEE ERQRQLEMSA EMSRAQARAE EEIGEKLHRT VQTLEIQRQQ AIAELEREKE LKSEEMQTVQ LQQSFLSEKD QEKAKLEQLF REEQQRQQQQ SMEEARRRQH EELQQLEQQR NQRLREQLQL HSEEVTASQV |
| | | | | | AATKTLPNGR EPEHSFDGLR AGILSAEELQ ELARREDVRH LLLKATNEKL LSPGTALILL DPVRNRRLTV PELHHKLLSA YTGQQISLFQ HGIRLLEAQI SHRVPVDVAY RVLADPSDDT NLTYLQLLER LLPLTDKAAK ARDVFEKATV TVTIWEIINS LLRQFRTGRI TVVEEQEQKG VPAAELLESR QRGERSVRDV RGANVIAGVW YNALKKDLLP QAGTGHIIDP AVRAGLVGPE AVTGYRDPYT KKGLIPREQG GGIVDPSKSH GCLDEETSRA YSDPSTGEPA PDQLTGLSLL QEELYSELQA VPVGGFKGRT YFTAEQRQEL VEKVIKILIT ERLSFSGLRA GVLSRAQFEQ LSELGSVRTL YLEDTKEKVS RATTAALLLE PVRNQRLYVH ELHEQLLSAE SGSTISLFQA GIRLLEAQIA HRVPVDVAYQ VLADPSDDTK LTYRQLLERC LPLKGAEKAE EETRRAFEET GGSTMSLWEV RAQLMADFQA IIIEIIEKTE DYVRRRLTAE ETYNLLREGT SAWCYLYGTG RQTLSIYQAL RLLLEAQAAT RLTVDEAVRK LLSAERAVTG SLFQAMKKEL DAQLATGGIV | DALDGPAAEA RKVSAQRLQE RLAQGHTTVD YLQGRSSIAG SVYAALQRQL EAQAASGFLL NEAVKEGVVG ERAVTGYKDP AMQKGLIVRE ATGGVIDPVH RRGYFDEEMN KGFFDPNTHE CVEDPETGLC GGELVYTDSE SAPFGKFQGK EYFTAEQRRD TVEKIIKIII RLCFEGLRSL VIDRELYQQL AEVDTVRRAL LEEAGQKLSI SDMAVALLEA ATSARLTVDE FHEKLLSAEK GQSVSLFQAL LRLLDAQLST RVPLDVACAR LSAPRADAKA TYGELQQRCR PLSEKAARAR RETFEKTPVE VTVWELISSE LRQFRTGKVT IVEEVETLRQ PVPASELLAS LKDGKTTVKD LQGSGCLAGI IYEAMRRGLL AQAATGFLVD EAVKAGWGP KAVTGYRDPY MQKGLVLRQH TGGIIDPVHS RGYFSEEMNR GFFDPNTHEN VEDPETGLRL VVETTQVYTE QIDIPGGGSH MQSDLIPEEQ GRVTKERMII IIRQQGLASY DLFEARIISL RSLREALEAE SVAGVYLPGS KKGLLSAEVA GFLLDPVKGE GLVGPELHDR YRDPYTEQTI IPTEEALRLL DPRLGFHLPL |
| | | | | | EVAYQRGYLN SEVRSYVDPS LRRCRRDDGT RKLTFRGLRK QVMDEATALQ VTKNLQKFLE DATKERLSVY GTAFELLEAQ KGLKLTVEEA KDKLLSAERA KLISLFQAMK RLLEAQIATG LPVEVAYKRG TDPSDDTKGF YLQLMERCIT LKEKKRERKT WIVDPETGK GLIDHQTYLE ITISSSDGVV RQYDIDDAIA QYRAGTLSIT GGFRSRSSSV AVSRTQLASW AGILDTETLE NLVDNITGQR IIDPSTGERF VDKIMVDRIN DPRTKTKMSA YEAGQRFLEV DTPGRVPLDE TAQKLRDVGA KLKISYKDAL LRLLEAAAQS SGSGSTAGSR SRRGSFDATG SYSSSGYGRR GPESAVA | KDTHDQLSEP TDERLSYTQL GQLLLPLSDA QITMEELVRS LREGLTSIEE GTSCIAGVFV QAMKKGIIRP AATGYVIDPI VRMGIVGPEF VTGYKDPYSG KGLILKDHGI GIIDPEESHR LFDEEMNEIL FDPNTEENLT DPQTGLCLLP SSKSSVRKRR EMSVYEAYRK LSEQECEWEE KSMIIDRRSG KNLIDRSALD EFADMLSGNA GSSSSYPISP SDPTEETGPV KVSITEAMHR LLEAQACTGG PVTDAVNKGL LAQKAFCGFE AQALKKGWLY QYLTGGLIEP ALQRGTVDAR YSKYLTCPKT DRSMVEEGTG TKGYYSPYSV TGSRTGSRAG SGFSMTFSSS YASGSSASLG |
| 71 | PLEC | 5 | Q151495 | Entry version 224 (18 Sep. 2019) Sequence version 3 (14 Oct. 2008) | MEPSGSLFPS AAVWHWRRGR DRVQKKTFTK RHISDLYEDL EVLSGDSLPR QNVQIALDYL RNDDIADGNP ILHFQISDIQ KEKLLLWSQR DNFTSSWRDG KPLLIDMNKV QAFSVAERDL VDVPQPDEKS AMPRVPDVQD WQEYRELVLL AFEERRFPSS FLKFKEMELP GIYQSLEGAV YHPLDVEKEW EKQLRSEFER LQMEAGLCEE SDVRLLAAGK | LVVVGHVVTL RWAQDEQDER WVNKHLIKAQ RDGHNLISLL EKGRMRFHKL RHRQVKLVNI KLTLGLIWTI VSGQSEDMTA MVEGYQGLRC RLFNAIIHRH YRQTNLENLD GVTRLLDPED IITYVSSLYD GVRANELQLR LLQWMRHHTA FEEIEILWSQ AKEADKNRSK QAGQLKVPPG GKLHVAILER LECLQRIVTK QLNQADALLQ VPQRAGEVER |
| | | | | | DLDKADSMIR DGRHPQGEQM LVAIRTEYNL TQVAQVTLQS TLRYLQDLLA GAEWGVDLPS LHQSIEEFRA QLSPATRGAY YAKLLNSSKA WLNEKEEEEV GFDWSDRNTN LMRELELKEK RLLREDHPAR QTQWSWMLQL NAAYFQFFSD LQEALRRKYS EDLLQDAQDE LSGLAKRAKA HPMRGRLPLL TVHKGDECQL VLSSSGSEAA PNQEAQEAVT TLWHQLHVDM RDVQLIRSWS EQRQALHSLE QDAGGFGPED CSHHYQQLLQ RCQRCISELK TRTVHRLRLP QRIAEQQKAQ ARLSAEAEKV PTLRSELELT SAIYLEKLKT AEEVLRAHEE TLPELEATKA AQQPTFDALR ERLQQRHGER VAQLLERWQA ELEQLGRQLR AWLQDARRRQ SQAVREQLRQ HGEKVEECQR DYELQLVTYK KKPKVQSGSE THYSELTTLT LRRMEEEERL RLAEVEAALE QAKAQAEREA VVRREEAAVD ELQQLRQSSE EAAERSRLRI LEATERQRGG RAEEAEAQKR RQVQDESQRK VKAEAEAARE LRLQAEEAER RQVQVALETA KRASFAEKTA VAVAQLREEA ERAREEAERE | LLFNDVQTLK YRRVYRLHER RLKAGVAAPA VQRRPELEDS WVEENQHRVD VEAQLGSHRG KIERARSDEG RDCLGRLDLQ RLRSLESLHS FVAAATKELM MTAKKESYSA KIKELQNAGD PTVESFQAAL CCCIEAHLKE VREAEGQLQK CDRSATVTRL KEQLNEYKGH VVQLKPRHPA AVCDYKQVEV VGPAQPSHWK VPSVCFLVPP RLEAQHQALV KSLLAWQSLR LATFRTLKPE LHYQAFLRDS RLMAEREYGS SLEQGAQEES DIRLQLEACE LDKEPARECA AEVEGLGKGV LALPEPSPAA LGKLEQVRSL ISLVIRGTQG QLKEAQAVPA SLKKLRAQAE DELRGAQEVG DVEVERWRER VLAQTDVRQR YYRESADPLG EQIQAMPLAD EQALLEEIER FAKQYINAIK AQLEPVASPA SVIQEYVDLR SQYIKFISET AEQQRAEERE KQRQLAEAHA KELQQRMQEE AQQQKRSIQE AEIQAKARQA EEEIRVVRLQ AEGELQALRA QAQEEAERLR RQAEVELASR KQRALQALEE RLRQAEVERA QRSAEAELQS QLERSLQEEH ERRAQQQAEA LERWQLKANE |
| | | | | | ALRLRLQAEE EAEKQKEEAE EQAVRQRELA AEGTAQQRLA ETEQGEQQRQ REAAAATQKR RAEMEVLLAS TSEKSKQRLE EEAARLRALA EEDAARQRAE AIGEATRLKT AENERLRRLA EEQAAQHKAD ASDSELERQK RQVEEEILAL KAELELELGR SKEQAELEAA RRRREAEERV ARQRKAALEE ARRLRERAEQ EAAQKRLQAE KEQELQQTLQ GEAEAARRAA EREAAQSRRQ AEEQAQARAQ EAEQEAARRA QAADAEMEKH KAQVEQELTT QKNLLDEELQ RQRSQVEEEL SKLKARIEAE NTQRFLQEEA ARLSVAAQEA LAQQRALAEK EATRLKAEAE EQARRLQEDK TQGFQRTLEA EAERLKLRVA EDAQRFRKQA ELATQEKVTL SDHDAERLRE KLQQEAKLLQ QEQLLQETQA SLLQRERFIE QDEVAKAQQL MEQERQRLVA EAEEGVRRKQ RQQEELLAEE LEEQHRAALA AATKTLPNGR EPEHSFDGLR AGILSAEELQ ELARREDVRH LLLKATNEKL LSPGTALILL DPVRNRRLTV PELHHKLLSA YTGQQISLFQ HGIRLLEAQI SHRVPVDVAY | VAQQKSLAQA REARRRGKAE EQELEKQRQL AEQELIRLRA LLEEELARLQ QELEAELAKV KARAEEESRS AEAGRFRELA EEAKRQRQLA AERVLAEKLA EAEIALKEKE EDEAFQRRRL IEERLAQLRK GLVEDTLRQR KASFEKAAAG IRSNAEDTLR RQRQLAAEEE QKSLAAEEEA VERLKAKVEE ESARQLQLAQ EKAHAFAVQQ QEQSVLDQLR EEAEEARVQA VEEAERLKQS AQAAAEKLRK QAEQAALRQK KKFAEQTLRQ LRLQLEETDH RLKAEATEAA FSVRVQMEEL NRALILRDKD EKMKQVAEEA ARLRQLAEED MLKEKMQAVQ LLQQQKELAQ EQMAQQLAEE ERQRQLEMSA EMSRAQARAE EEIGEKLHRT VQTLEIQRQQ AIAELEREKE LKSEEMQTVQ LQQSFLSEKD QEKAKLEQLF REEQQRQQQQ SMEEARRRQH EELQQLEQQR NQRLREQLQL HSEEVTASQV DALDGPAAEA RKVSAQRLQE RLAQGHTTVD YLQGRSSIAG SVYAALQRQL EAQAASGFLL NEAVKEGVVG ERAVTGYKDP AMQKGLIVRE ATGGVIDPVH RRGYFDEEMN |
| | | | | | RVLADPSDDT NLTYLQLLER LLPLTDKAAK ARDVFEKATV TVTIWEIINS LLRQFRTGRI TVVEEQEQKG VPAAELLESR QRGERSVRDV RGANVIAGVW YNALKKDLLP QAGTGHIIDP AVRAGLVGPE AVTGYRDPYT KKGLIPREQG GGIVDPSKSH GCLDEETSRA YSDPSTGEPA PDQLTGLSLL QEELYSELQA VPVGGFKGRT YFTAEQRQEL VEKVIKILIT ERLSFSGLRA GVLSRAQFEQ LSELGSVRTL YLEDTKEKVS RATTAALLLE PVRNQRLYVH ELHEQLLSAE SGSTISLFQA GIRLLEAQIA HRVPVDVAYQ VLADPSDDTK LTYRQLLERC LPLKGAEKAE EETRRAFEET GGSTMSLWEV RAQLMADFQA IIIEIIEKTE DYVRRRLTAE ETYNLLREGT SAWCYLYGTG RQTLSIYQAL RLLLEAQAAT RLTVDEAVRK LLSAERAVTG SLFQAMKKEL DAQLATGGIV EVAYQRGYLN SEVRSYVDPS LRRCRRDDGT RKLTFRGLRK QVMDEATALQ VTKNLQKFLE DATKERLSVY GTAFELLEAQ KGLKLTVEEA KDKLLSAERA KLISLFQAMK | KGFFDPNTHE CVEDPETGLC GGELVYTDSE SAPFGKFQGK EYFTAEQRRD TVEKIIKIII RLCFEGLRSL VIDRELYQQL AEVDTVRRAL LEEAGQKLSI SDMAVALLEA ATSARLTVDE FHEKLLSAEK GQSVSLFQAL LRLLDAQLST RVPLDVACAR LSAPRADAKA TYGELQQRCR PLSEKAARAR RETFEKTPVE VTVWELISSE LRQFRTGKVT IVEEVETLRQ PVPASELLAS LKDGKTTVKD LQGSGCLAGI IYEAMRRGLL AQAATGFLVD EAVKAGWGP KAVTGYRDPY MQKGLVLRQH TGGIIDPVHS RGYFSEEMNR GFFDPNTHEN VEDPETGLRL WETTQVYTE QIDIPGGGSH MQSDLIPEEQ GRVTKERMII IIRQQGLASY DLFEARIISL RSLREALEAE SVAGVYLPGS KKGLLSAEVA GFLLDPVKGE GLVGPELHDR YRDPYTEQTI IPTEEALRLL DPRLGFHLPL KDTHDQLSEP TDERLSYTQL GQLLLPLSDA QITMEELVRS LREGLTSIEE GTSCIAGVFV QAMKKGIIRP AATGYVIDPI VRMGIVGPEF VTGYKDPYSG KGLILKDHGI |
| | | | | | RLLEAQIATG LPVEVAYKRG TDPSDDTKGF YLQLMERCIT LKEKKRERKT WIVDPETGK GLIDHQTYLE ITISSSDGVV RQYDIDDAIA QYRAGTLSIT GGFRSRSSSV AVSRTQLASW AGILDTETLE NLVDNITGQR IIDPSTGERF VDKIMVDRIN DPRTKTKMSA YEAGQRFLEV DTPGRVPLDE TAQKLRDVGA KLKISYKDAL LRLLEAAAQS SGSGSTAGSR SRRGSFDATG SYSSSGYGRR GPESAVA | GIIDPEESHR LFDEEMNEIL FDPNTEENLT DPQTGLCLLP SSKSSVRKRR EMSVYEAYRK LSEQECEWEE KSMIIDRRSG KNLIDRSALD EFADMLSGNA GSSSSYPISP SDPTEETGPV KVSITEAMHR LLEAQACTGG PVTDAVNKGL LAQKAFCGFE AQALKKGWLY QYLTGGLIEP ALQRGTVDAR YSKYLTCPKT DRSMVEEGTG TKGYYSPYSV TGSRTGSRAG SGFSMTFSSS YASGSSASLG |
| 72 | PLEC | 6 | Q151496 | Entry version 224 (18 Sep. 2019) Sequence version 3 (14 Oct. 2008) | MSGAGGAFAS WLDGGCEPAR VDERDRVQKK IKAQRHISDL ISLLEVLSGD FHKLQNVQIA LVNIRNDDIA IWTIILHFQI DMTAKEKLLL GLRCDNFTSS IHRHKPLLID ENLDQAFSVA DPEDVDVPQP SLYDAMPRVP LQLRWQEYRE HHTAAFEERR LWSQFLKFKE NRSKGIYQSL VPPGYHPLDV ILEREKQLRS IVTKLQMEAG ALLQSDVRLL EVERDLDKAD QTLKDGRHPQ LHERLVAIRT AAPATQVAQV LEDSTLRYLQ HRVDGAEWGV SHRGLHQSIE SDEGQLSPAT LDLQYAKLLN SLHSFVAAAT EEEVGFDWSD | PREVLLERPC RGYLYQQLCC TFTKWVNKHL YEDLRDGHNL SLPREKGRMR LDYLRHRQVK DGNPKLTLGL SDIQVSGQSE WSQRMVEGYQ WRDGRLFNAI MNKVYRQTNL ERDLGVTRLL DEKSIITYVS DVQDGVRANE LVLLLLQWMR FPSSFEEIEI MELPAKEADK EGAVQAGQLK EKEWGKLHVA EFERLECLQR LCEEQLNQAD AAGKVPQRAG SMIRLLFNDV GEQMYRRVYR EYNLRLKAGV TLQSVQRRPE DLLAWVEENQ DLPSVEAQLG EFRAKIERAR RGAYRDCLGR SSKARLRSLE KELMWLNEKE RNTNMTAKKE |
| | | | | | SYSALMRELE NAGDRLLRED QAALQTQWSW HLKENAAYFQ QLQKLQEALR VTRLEDLLQD YKGHLSGLAK RHPAHPMRGR QVEVTVHKGD SHWKVLSSSG LVPPPNQEAQ QALVTLWHQL QSLRRDVQLI LKPEEQRQAL LRDSQDAGGF EYGSCSHHYQ QEESRCQRCI EACETRTVHR RECAQRIAEQ GKGVARLSAE SPAAPTLRSE VRSLSAIYLE GTQGAEEVLR AVPATLPELE AQAEAQQPTF QEVGERLQQR HGERDVEVER VRQRELEQLG DPLGAWLQDA PLADSQAVRE EIERHGEKVE NAIKDYELQL ASPAKKPKVQ VDLRTHYSEL ISETLRRMEE EERERLAEVE EAHAQAKAQA MQEEWRREE SIQEELQQLR ARQAEAAERS VRLQLEATER ALRARAEEAE ERLRRQVQDE LASRVKAEAE ALEELRLQAE VERARQVQVA ELQSKRASFA QEEHVAVAQL QAEAERAREE KANEALRLRL LAQAEAEKQK GKAEEQAVRQ QRQLAEGTAQ RLRAETEQGE ARLQREAAAA LAKVRAEMEV ESRSTSEKSK RELAEEAARL RQLAEEDAAR EKLAAIGEAT | LKEKKIKELQ HPARPTVESF MLQLCCCIEA FFSDVREAEG RKYSCDRSAT AQDEKEQLNE RAKAVVQLKP LPLLAVCDYK ECQLVGPAQP SEAAVPSVCF EAVTRLEAQH HVDMKSLLAW RSWSLATFRT HSLELHYQAF GPEDRLMAER QLLQSLEQGA SELKDIRLQL LRLPLDKEPA QKAQAEVEGL AEKVLALPEP LELTLGKLEQ KLKTISLVIR AHEEQLKEAQ ATKASLKKLR DALRDELRGA WRERVAQLLE RWQAVLAQTD RQLRYYRESA RRRQEQIQAM QLRQEQALLE ECQRFAKQYI VTYKAQLEPV SGSESVIQEY TTLTSQYIKF EERLAEQQRA AALEKQRQLA EREAKELQQR AAVDAQQQKR QSSEAEIQAK RLRIEEEIRV QRGGAEGELQ AQKRQAQEEA SQRKRQAEVE AAREKQRALQ EAERRLRQAE LETAQRSAEA EKTAQLERSL REEAERRAQQ AERELERWQL QAEEVAQQKS EEAEREARRR RELAEQELEK QRLAAEQELI QQRQLLEEEL TQKRQELEAE LLASKARAEE QRLEAEAGRF RALAEEAKRQ QRAEAERVLA RLKTEAEIAL |
| | | | | | KEKEAENERL RRRLEEQAAQ QLRKASDSEL LRQRRQVEEE AAAGKAELEL DTLRSKEQAE AEEERRRREA EEEAARQRKA KVEEARRLRE QLAQEAAQKR AVQQKEQELQ DQLRGEAEAA RVQAEREAAQ LKQSAEEQAQ KLRKEAEQEA LRQKQAADAE TLRQKAQVEQ ETDHQKNLLD TEAARQRSQV MEELSKLKAR RDKDNTQRFL AEEAARLSVA AEEDLAQQRA QAVQEATRLK ELAQEQARRL LAEETQGFQR EMSAEAERLK ARAEEDAQRF LHRTELATQE QRQQSDHDAE REKEKLQQEA QTVQQEQLLQ SEKDSLLQRE EQLFQDEVAK QQQQMEQERQ RRQHEAEEGV EQQRRQQEEL QLQLLEEQHR ASQVAATKTL AAEAEPEHSF RLQEAGILSA TTVDELARRE SIAGLLLKAT QRQLLSPGTA GFLLDPVRNR GVVGPELHHK YKDPYTGQQI IVREHGIRLL DPVHSHRVPV EEMNRVLADP NTHENLTYLQ TGLCLLPLTD TDSEARDVFE FQGKTVTIWE QRRDLLRQFR KIIITWEEQ LRSLVPAAEL YQQLQRGERS RRALRGANVI KLSIYNALKK | RRLAEDEAFQ HKADIEERLA ERQKGLVEDT ILALKASFEK ELGRIRSNAE LEAARQRQLA EERVQKSLAA ALEEVERLKA RAEQESARQL LQAEEKAHAF QTLQQEQSVL RRAAEEAEEA SRRQVEEAER ARAQAQAAAE ARRAQAEQAA MEKHKKFAEQ ELTTLRLQLE EELQRLKAEA EEELFSVRVQ IEAENRALIL QEEAEKMKQV AQEAARLRQL LAEKMLKEKM AEAELLQQQK QEDKEQMAQQ TLEAERQRQL LRVAEMSRAQ RKQAEEIGEK KVTLVQTLEI RLREAIAELE KLLQLKSEEM ETQALQQSFL RFIEQEKAKL AQQLREEQQR RLVASMEEAR RRKQEELQQL LAEENQRLRE AALAHSEEVT PNGRDALDGP DGLRRKVSAQ EELQRLAQGH DVRHYLQGRS NEKLSVYAAL LILLEAQAAS RLTVNEAVKE LLSAERAVTG SLFQAMQKGL EAQIATGGVI DVAYRRGYFD SDDTKGFFDP LLERCVEDPE KAAKGGELVY KATVSAPFGK IINSEYFTAE TGRITVEKII EQKGRLCFEG LESRVIDREL VRDVAEVDTV AGVWLEEAGQ DLLPSDMAVA |
| | | | | | LLEAQAGTGH TVDEAVRAGL SAEKAVTGYR FQALKKGLIP QLSTGGIVDP ACARGCLDEE DAKAYSDPST QRCRPDQLTG ARARQEELYS TPVEVPVGGF ISSEYFTAEQ GKVTVEKVIK TLRQERLSFS LLASGVLSRA TVKDLSELGS LAGIYLEDTK RGLLRATTAA FLVDPVRNQR VVGPELHEQL RDPYSGSTIS LRQHGIRLLE PVHSHRVPVD EMNRVLADPS THENLTYRQL GLRLLPLKGA VYTEEETRRA GGSHGGSTMS PEEQRAQLMA RMIIIIIEII LASYDYVRRR IISLETYNLL LEAESAWCYL LPGSRQTLSI AEVARLLLEA VKGERLTVDE LHDRLLSAER EQTISLFQAM LRLLDAQLAT HLPLEVAYQR LSEPSEVRSY YTQLLRRCRR LSDARKLTFR LVRSQVMDEA SIEEVTKNLQ GVFVDATKER IIRPGTAFEL IDPIKGLKLT GPEFKDKLLS PYSGKLISLF DHGIRLLEAQ ESHRLPVEVA NEILTDPSDD ENLTYLQLME CLLPLKEKKR RKRRVVIVDP AYRKGLIDHQ EWEEITISSS RRSGRQYDID SALDQYRAGT SGNAGGFRSR | IIDPATSARL VGPEFHEKLL DPYTGQSVSL REQGLRLLDA SKSHRVPLDV TSRALSAPRA GEPATYGELQ LSLLPLSEKA ELQARETFEK KGRTVTVWEL RQELLRQFRT ILITIVEEVE GLRAPVPASE QFEQLKDGKT VRTLLQGSGC EKVSIYEAMR LLLEAQAATG LYVHEAVKAG LSAEKAVTGY LFQAMQKGLV AQIATGGIID VAYQRGYFSE DDTKGFFDPN LERCVEDPET EKAEWETTQ FEETQIDIPG LWEVMQSDLI DFQAGRVTKE EKTEIIRQQG LTAEDLFEAR REGTRSLREA YGTGSVAGVY YQALKKGLLS QAATGFLLDP AVRKGLVGPE AVTGYRDPYT KKELIPTEEA GGIVDPRLGF GYLNKDTHDQ VDPSTDERLS DDGTGQLLLP GLRKQITMEE TALQLREGLT KFLEGTSCIA LSVYQAMKKG LEAQAATGYV VEEAVRMGIV AERAVTGYKD QAMKKGLILK IATGGIIDPE YKRGLFDEEM TKGFFDPNTE RCITDPQTGL ERKTSSKSSV ETGKEMSVYE TYLELSEQEC DGWKSMIID DAIAKNLIDR LSITEFADML SSSVGSSSSY |
| | | | | | PISPAVSRTQ TGPVAGILDT AMHRNLVDNI CTGGIIDPST NKGLVDKIMV CGFEDPRTKT GWLYYEAGQR LIEPDTPGRV VDARTAQKLR CPKTKLKISY EGTGLRLLEA PYSVSGSGST SRAGSRRGSF FSSSSYSSSG ASLGGPESAV | LASWSDPTEE ETLEKVSITE TGQRLLEAQA GERFPVTDAV DRINLAQKAF KMSAAQALKK FLEVQYLTGG PLDEALQRGT DVGAYSKYLT KDALDRSMVE AAQSTKGYYS AGSRTGSRTG DATGSGFSMT YGRRYASGSS A |
| 73 | PLEC | 7 | Q151497 | Entry version 224 (18 Sep. 2019) Sequence version 3 (14 Oct. 2008) | MKIVPDERDR NKHLIKAQRH GHNLISLLEV GRMRFHKLQN RQVKLVNIRN TLGLIWTIIL GQSEDMTAKE EGYQGLRCDN FNAIIHRHKP QTNLENLDQA TRLLDPEDVD TYVSSLYDAM RANELQLRWQ QWMRHHTAAF EIEILWSQFL EADKNRSKGI GQLKVPPGYH LHVAILEREK CLQRIVTKLQ NQADALLQSD QRAGEVERDL FNDVQTLKDG RVYRLHERLV KAGVAAPATQ RRPELEDSTL EENQHRVDGA AQLGSHRGLH ERARSDEGQL CLGRLDLQYA RSLESLHSFV NEKEEEEVGF AKKESYSALM KELQNAGDRL VESFQAALQT CIEAHLKENA EAEGQLQKLQ RSATVTRLED QLNEYKGHLS QLKPRHPAHP CDYKQVEVTV PAQPSHWKVL SVCFLVPPPN EAQHQALVTL LLAWQSLRRD | VQKKTFTKWV ISDLYEDLRD LSGDSLPREK VQIALDYLRH DDIADGNPKL HFQISDIQVS KLLLWSQRMV FTSSWRDGRL LLIDMNKVYR FSVAERDLGV VPQPDEKSII PRVPDVQDGV EYRELVLLLL EERRFPSSFE KFKEMELPAK YQSLEGAVQA PLDVEKEWGK QLRSEFERLE MEAGLCEEQL VRLLAAGKVP DKADSMIRLL RHPQGEQMYR AIRTEYNLRL VAQVTLQSVQ RYLQDLLAWV EWGVDLPSVE QSIEEFRAKI SPATRGAYRD KLLNSSKARL AAATKELMWL DWSDRNTNMT RELELKEKKI LREDHPARPT QWSWMLQLCC AYFQFFSDVR EALRRKYSCD LLQDAQDEKE CLAKRAKAVV MRGRLPLLAV HKGDECQLVG SSSGSEAAVP QEAQEAVTRL WHQLHVDMKS VQLIRSWSLA |
| | | | | | TFRTLKPEEQ YQAFLRDSQD MAEREYGSCS EQGAQEESRC RLQLEACETR KEPARECAQR VEGLGKGVAR LPEPSPAAPT KLEQVRSLSA LVIRGTQGAE KEAQAVPATL KKLRAQAEAQ LRGAQEVGER EVERWRERVA AQTDVRQREL RESADPLGAW IQAMPLADSQ ALLEEIERHG KQYINAIKDY LEPVASPAKK IQEYVDLRTH YIKFISETLR QQRAEERERL RQLAEAHAQA LQQRMQEEW QQKRSIQEEL IQAKARQAEA EIRVVRLQLE GELQALRARA QEEAERLRRQ AEVELASRVK RALQALEELR RQAEVERARQ SAEAELQSKR ERSLQEEHVA RAQQQAEAER RWQLKANEAL QQKSLAQAEA ARRRGKAEEQ ELEKQRQLAE QELIRLRAET EEELARLQRE LEAELAKVRA RAEEESRSTS AGRFRELAEE AKRQRQLAEE RVLAEKLAAI EIALKEKEAE EAFQRRRLEE ERLAQLRKAS VEDTLRQRRQ SFEKAAAGKA SNAEDTLRSK RQLAAEEERR SLAAEEEAAR RLKAKVEEAR ARQLQLAQEA AHAFAVQQKE QSVLDQLRGE AEEARVQAER | RQALHSLELH AGGFGPEDRL HHYQQLLQSL QRCISELKDI TVHRLRLPLD IAEQQKAQAE LSAEAEKVLA LRSELELTLG IYLEKLKTIS EVLRAHEEQL PELEATKASL QPTFDALRDE LQQRHGERDV QLLERWQAVL EQLGRQLRYY LQDARRRQEQ AVREQLRQEQ EKVEECQRFA ELQLVTYKAQ PKVQSGSESV YSELTTLTSQ RMEEEERLAE AEVEAALEKQ KAQAEREAKE RREEAAVDAQ QQLRQSSEAE AERSRLRIEE ATERQRGGAE EEAEAQKRQA VQDESQRKRQ AEAEAAREKQ LQAEEAERRL VQVALETAQR ASFAEKTAQL VAQLREEAER AREEAERELE RLRLQAEEVA EKQKEEAERE AVRQRELAEQ GTAQQRLAAE EQGEQQRQLL AAAATQKRQE EMEVLLASKA EKSKQRLEAE AARLRALAEE DAARQRAEAE GEATRLKTEA NERLRRLAED QAAQHKADIE DSELERQKGL VEEEILALKA ELELELGRIR EQAELEAARQ RREAEERVQK QRKAALEEVE RLRERAEQES AQKRLQAEEK QELQQTLQQE AEAARRAAEE EAAQSRRQVE |
| | | | | | EAERLKQSAE AAAEKLRKEA EQAALRQKQA FAEQTLRQKA LQLEETDHQK KAEATEAARQ VRVQMEELSK ALILRDKDNT MKQVAEEAAR LRQLAEEDLA KEKMQAVQEA QQQKELAQEQ MAQQLAEETQ QRQLEMSAEA SRAQARAEED IGEKLHRTEL TLEIQRQQSD AELEREKEKL SEEMQTVQQE QSFLSEKDSL KAKLEQLFQD EQQRQQQQME EEARRRQHEA LQQLEQQRRQ RLREQLQLLE EEVTASQVAA LDGPAAEAEP VSAQRLQEAG AQGHTTVDEL QGRSSIAGLL YAALQRQLLS QAASGFLLDP AVKEGVVGPE AVTGYKDPYT QKGLIVREHG GGVIDPVHSH GYFDEEMNRV FFDPNTHENL EDPETGLCLL ELVYTDSEAR PFGKFQGKTV FTAEQRRDLL EKIIKIIITV CFEGLRSLVP DRELYQQLQR VDTVRRALRG EAGQKLSIYN MAVALLEAQA SARLTVDEAV EKLLSAEKAV SVSLFQALKK LLDAQLSTGG PLDVACARGC APRADAKAYS GELQQRCRPD SEKAARARQE TFEKTPVEVP VWELISSEYF QFRTGKVTVE EEVETLRQER | EQAQARAQAQ EQEAARRAQA ADAEMEKHKK QVEQELTTLR NLLDEELQRL RSQVEEELFS LKARIEAENR QRFLQEEAEK LSVAAQEAAR QQRALAEKML TRLKAEAELL ARRLQEDKEQ GFQRTLEAER ERLKLRVAEM AQRFRKQAEE ATQEKVTLVQ HDAERLREAI QQEAKLLQLK QLLQETQALQ LQRERFIEQE EVAKAQQLRE QERQRLVASM EEGVRRKQEE QEELLAEENQ EQHRAALAHS TKTLPNGRDA EHSFDGLRRK ILSAEELQRL ARREDVRHYL LKATNEKLSV PGTALILLEA VRNRRLTVNE LHHKLLSAER GQQISLFQAM IRLLEAQIAT RVPVDVAYRR LADPSDDTKG TYLQLLERCV PLTDKAAKGG DVFEKATVSA TIWEIINSEY RQFRTGRITV VEEQEQKGRL AAELLESRVI GERSVRDVAE ANVIAGVWLE ALKKDLLPSD GTGHIIDPAT RAGLVGPEFH TGYRDPYTGQ GLIPREQGLR IVDPSKSHRV LDEETSRALS DPSTGEPATY QLTGLSLLPL ELYSELQARE VGGFKGRTVT TAEQRQELLR KVIKILITIV LSFSGLRAPV |
| | | | | | PASELLASGV DGKTTVKDLS GSGCLAGIYL EAMRRGLLRA AATGFLVDPV VKAGVVGPEL VTGYRDPYSG KGLVLRQHGI GIIDPVHSHR YFSEEMNRVL FDPNTHENLT DPETGLRLLP ETTQVYTEEE DIPGGGSHGG SDLIPEEQRA VTKERMIIII RQQGLASYDY FEARIISLET LREALEAESA AGVYLPGSRQ GLLSAEVARL LLDPVKGERL VGPELHDRLL DPYTEQTISL TEEALRLLDA RLGFHLPLEV THDQLSEPSE ERLSYTQLLR LLLPLSDARK TMEELVRSQV EGLTSIEEVT SCIAGVFVDA MKKGIIRPGT TGYVIDPIKG MGIVGPEFKD GYKDPYSGKL LILKDHGIRL IDPEESHRLP DEEMNEILTD PNTEENLTYL QTGLCLLPLK KSSVRKRRVV SVYEAYRKGL EQECEWEEIT MIIDRRSGRQ LIDRSALDQY ADMLSGNAGG SSSYPISPAV PTEETGPVAG SITEAMHRNL EAQACTGGII TDAVNKGLVD QKAFCGFEDP ALKKGWLYYE LTGGLIEPDT QRGTVDARTA KYLTCPKTKL SMVEEGTGLR GYYSPYSVSG SRTGSRAGSR | LSRAQFEQLK ELGSVRTLLQ EDTKEKVSIY TTAALLLEAQ RNQRLYVHEA HEQLLSAEKA STISLFQAMQ RLLEAQIATG VPVDVAYQRG ADPSDDTKGF YRQLLERCVE LKGAEKAEW TRRAFEETQI STMSLWEVMQ QLMADFQAGR IEIIEKTEII VRRRLTAEDL YNLLREGTRS WCYLYGTGSV TLSIYQALKK LLEAQAATGF TVDEAVRKGL SAERAVTGYR FQAMKKELIP QLATGGIVDP AYQRGYLNKD VRSYVDPSTD RCRRDDGTGQ LTFRGLRKQI MDEATALQLR KNLQKFLEGT TKERLSVYQA AFELLEAQAA LKLTVEEAVR KLLSAERAVT ISLFQAMKKG LEAQIATGGI VEVAYKRGLF PSDDTKGFFD QLMERCITDP EKKRERKTSS IVDPETGKEM IDHQTYLELS ISSSDGVVKS YDIDDAIAKN RAGTLSITEF FRSRSSSVGS SRTQLASWSD ILDTETLEKV VDNITGQRLL DPSTGERFPV KIMVDRINLA RTKTKMSAAQ AGQRFLEVQY PGRVPLDEAL QKLRDVGAYS KISYKDALDR LLEAAAQSTK SGSTAGSRTG RGSFDATGSG |
| | | | | | FSMTFSSSSY SGSSASLGGP | SSSGYGRRYA ESAVA |
| 74 | PLEC | 8 | Q151498 | Entry version 224 (18 Sep. 2019) Sequence version 3 (14 Oct. 2008) | MDPSRAIQNE VQKKTFTKWV ISDLYEDLRD LSGDSLPREK VQIALDYLRH DDIADGNPKL HFQISDIQVS KLLLWSQRMV FTSSWRDGRL LLIDMNKVYR FSVAERDLGV VPQPDEKSII PRVPDVQDGV EYRELVLLLL EERRFPSSFE KFKEMELPAK YQSLEGAVQA PLDVEKEWGK QLRSEFERLE MEAGLCEEQL VRLLAAGKVP DKADSMIRLL RHPQGEQMYR AIRTEYNLRL VAQVTLQSVQ RYLQDLLAWV EWGVDLPSVE QSIEEFRAKI SPATRGAYRD KLLNSSKARL AAATKELMWL DWSDRNTNMT RELELKEKKI LREDHPARPT QWSWMLQLCC AYFQFFSDVR EALRRKYSCD LLQDAQDEKE CLAKRAKAVV MRGRLPLLAV HKGDECQLVG SSSGSEAAVP QEAQEAVTRL WHQLHVDMKS VQLIRSWSLA RQALHSLELH AGGFGPEDRL HHYQQLLQSL QRCISELKDI TVHRLRLPLD IAEQQKAQAE LSAEAEKVLA LRSELELTLG IYLEKLKTIS EVLRAHEEQL PELEATKASL QPTFDALRDE | ISSLKDERDR NKHLIKAQRH GHNLISLLEV GRMRFHKLQN RQVKLVNIRN TLGLIWTIIL GQSEDMTAKE EGYQGLRCDN FNAIIHRHKP QTNLENLDQA TRLLDPEDVD TYVSSLYDAM RANELQLRWQ QWMRHHTAAF EIEILWSQFL EADKNRSKGI GQLKVPPGYH LHVAILEREK CLQRIVTKLQ NQADALLQSD QRAGEVERDL FNDVQTLKDG RVYRLHERLV KAGVAAPATQ RRPELEDSTL EENQHRVDGA AQLGSHRGLH ERARSDEGQL CLGRLDLQYA RSLESLHSFV NEKEEEEVGF AKKESYSALM KELQNAGDRL VESFQAALQT CIEAHLKENA EAEGQLQKLQ RSATVTRLED QLNEYKGHLS QLKPRHPAHP CDYKQVEVTV PAQPSHWKVL SVCFLVPPPN EAQHQALVTL LLAWQSLRRD TFRTLKPEEQ YQAFLRDSQD MAEREYGSCS EQGAQEESRC RLQLEACETR KEPARECAQR VEGLGKGVAR LPEPSPAAPT KLEQVRSLSA LVIRGTQGAE KEAQAVPATL KKLRAQAEAQ LRGAQEVGER |
| | | | | | LQQRHGERDV QLLERWQAVL EQLGRQLRYY LQDARRRQEQ AVREQLRQEQ EKVEECQRFA ELQLVTYKAQ PKVQSGSESV YSELTTLTSQ RMEEEERLAE AEVEAALEKQ KAQAEREAKE RREEAAVDAQ QQLRQSSEAE AERSRLRIEE ATERQRGGAE EEAEAQKRQA VQDESQRKRQ AEAEAAREKQ LQAEEAERRL VQVALETAQR ASFAEKTAQL VAQLREEAER AREEAERELE RLRLQAEEVA EKQKEEAERE AVRQRELAEQ GTAQQRLAAE EQGEQQRQLL AAAATQKRQE EMEVLLASKA EKSKQRLEAE AARLRALAEE DAARQRAEAE GEATRLKTEA NERLRRLAED QAAQHKADIE DSELERQKGL VEEEILALKA ELELELGRIR EQAELEAARQ RREAEERVQK QRKAALEEVE RLRERAEQES AQKRLQAEEK QELQQTLQQE AEAARRAAEE EAAQSRRQVE EQAQARAQAQ EQEAARRAQA ADAEMEKHKK QVEQELTTLR NLLDEELQRL RSQVEEELFS LKARIEAENR QRFLQEEAEK LSVAAQEAAR QQRALAEKML TRLKAEAELL ARRLQEDKEQ | EVERWRERVA AQTDVRQREL RESADPLGAW IQAMPLADSQ ALLEEIERHG KQYINAIKDY LEPVASPAKK IQEYVDLRTH YIKFISETLR QQRAEERERL RQLAEAHAQA LQQRMQEEW QQKRSIQEEL IQAKARQAEA EIRVVRLQLE GELQALRARA QEEAERLRRQ AEVELASRVK RALQALEELR RQAEVERARQ SAEAELQSKR ERSLQEEHVA RAQQQAEAER RWQLKANEAL QQKSLAQAEA ARRRGKAEEQ ELEKQRQLAE QELIRLRAET EEELARLQRE LEAELAKVRA RAEEESRSTS AGRFRELAEE AKRQRQLAEE RVLAEKLAAI EIALKEKEAE EAFQRRRLEE ERLAQLRKAS VEDTLRQRRQ SFEKAAAGKA SNAEDTLRSK RQLAAEEERR SLAAEEEAAR RLKAKVEEAR ARQLQLAQEA AHAFAVQQKE QSVLDQLRGE AEEARVQAER EAERLKQSAE AAAEKLRKEA EQAALRQKQA FAEQTLRQKA LQLEETDHQK KAEATEAARQ VRVQMEELSK ALILRDKDNT MKQVAEEAAR LRQLAEEDLA KEKMQAVQEA QQQKELAQEQ MAQQLAEETQ |
| | | | | | GFQRTLEAER ERLKLRVAEM AQRFRKQAEE ATQEKVTLVQ HDAERLREAI QQEAKLLQLK QLLQETQALQ LQRERFIEQE EVAKAQQLRE QERQRLVASM EEGVRRKQEE QEELLAEENQ EQHRAALAHS TKTLPNGRDA EHSFDGLRRK ILSAEELQRL ARREDVRHYL LKATNEKLSV PGTALILLEA VRNRRLTVNE LHHKLLSAER GQQISLFQAM IRLLEAQIAT RVPVDVAYRR LADPSDDTKG TYLQLLERCV PLTDKAAKGG DVFEKATVSA TIWEIINSEY RQFRTGRITV VEEQEQKGRL AAELLESRVI GERSVRDVAE ANVIAGVWLE ALKKDLLPSD GTGHIIDPAT RAGLVGPEFH TGYRDPYTGQ GLIPREQGLR IVDPSKSHRV LDEETSRALS DPSTGEPATY QLTGLSLLPL ELYSELQARE VGGFKGRTVT TAEQRQELLR KVIKILITIV LSFSGLRAPV LSRAQFEQLK ELGSVRTLLQ EDTKEKVSIY TTAALLLEAQ RNQRLYVHEA HEQLLSAEKA STISLFQAMQ RLLEAQIATG VPVDVAYQRG ADPSDDTKGF YRQLLERCVE LKGAEKAEW | QRQLEMSAEA SRAQARAEED IGEKLHRTEL TLEIQRQQSD AELEREKEKL SEEMQTVQQE QSFLSEKDSL KAKLEQLFQD EQQRQQQQME EEARRRQHEA LQQLEQQRRQ RLREQLQLLE EEVTASQVAA LDGPAAEAEP VSAQRLQEAG AQGHTTVDEL QGRSSIAGLL YAALQRQLLS QAASGFLLDP AVKEGVVGPE AVTGYKDPYT QKGLIVREHG GGVIDPVHSH GYFDEEMNRV FFDPNTHENL EDPETGLCLL ELVYTDSEAR PFGKFQGKTV FTAEQRRDLL EKIIKIIITV CFEGLRSLVP DRELYQQLQR VDTVRRALRG EAGQKLSIYN MAVALLEAQA SARLTVDEAV EKLLSAEKAV SVSLFQALKK LLDAQLSTGG PLDVACARGC APRADAKAYS GELQQRCRPD SEKAARARQE TFEKTPVEVP VWELISSEYF QFRTGKVTVE EEVETLRQER PASELLASGV DGKTTVKDLS GSGCLAGIYL EAMRRGLLRA AATGFLVDPV VKAGVVGPEL VTGYRDPYSG KGLVLRQHGI GIIDPVHSHR YFSEEMNRVL FDPNTHENLT DPETGLRLLP ETTQVYTEEE |
| | | | | | TRRAFEETQI STMSLWEVMQ QLMADFQAGR IEIIEKTEII VRRRLTAEDL YNLLREGTRS WCYLYGTGSV TLSIYQALKK LLEAQAATGF TVDEAVRKGL SAERAVTGYR FQAMKKELIP QLATGGIVDP AYQRGYLNKD VRSYVDPSTD RCRRDDGTGQ LTFRGLRKQI MDEATALQLR KNLQKFLEGT TKERLSVYQA AFELLEAQAA LKLTVEEAVR KLLSAERAVT ISLFQAMKKG LEAQIATGGI VEVAYKRGLF PSDDTKGFFD QLMERCITDP EKKRERKTSS IVDPETGKEM IDHQTYLELS ISSSDGVVKS YDIDDAIAKN RAGTLSITEF FRSRSSSVGS SRTQLASWSD ILDTETLEKV VDNITGQRLL DPSTGERFPV KIMVDRINLA RTKTKMSAAQ AGQRFLEVQY PGRVPLDEAL QKLRDVGAYS KISYKDALDR LLEAAAQSTK SGSTAGSRTG FSMTFSSSSY SSSGYGRRYA ESAVA | DIPGGGSHGG SDLIPEEQRA VTKERMIIII RQQGLASYDY FEARIISLET LREALEAESA AGVYLPGSRQ GLLSAEVARL LLDPVKGERL VGPELHDRLL DPYTEQTISL TEEALRLLDA RLGFHLPLEV THDQLSEPSE ERLSYTQLLR LLLPLSDARK TMEELVRSQV EGLTSIEEVT SCIAGVFVDA MKKGIIRPGT TGYVIDPIKG MGIVGPEFKD GYKDPYSGKL LILKDHGIRL IDPEESHRLP DEEMNEILTD PNTEENLTYL QTGLCLLPLK KSSVRKRRVV SVYEAYRKGL EQECEWEEIT MIIDRRSGRQ LIDRSALDQY ADMLSGNAGG SSSYPISPAV PTEETGPVAG SITEAMHRNL EAQACTGGII TDAVNKGLVD QKAFCGFEDP ALKKGWLYYE LTGGLIEPDT QRGTVDARTA KYLTCPKTKL SMVEEGTGLR GYYSPYSVSG SRTGSRAGSR RGSFDATGSG SGSSASLGGP |
| 75 | PLEC | 9 | Q151499 | Entry version 224 (18 Sep. 2019) Sequence version 3 (14 Oct. 2008) | MAGPLPDEQD KYKDERDRVQ HLIKAQRHIS NLISLLEVLS MRFHKLQNVQ VKLVNIRNDD GLIWTIILHF SEDMTAKEKL YQGLRCDNFT AIIHRHKPLL | FIQAYEEVRE KKTFTKWVNK DLYEDLRDGH GDSLPREKGR IALDYLRHRQ IADGNPKLTL QISDIQVSGQ LLWSQRMVEG SSWRDGRLFN IDMNKVYRQT |
| | | | | | NLENLDQAFS LLDPEDVDVP VSSLYDAMPR NELQLRWQEY MRHHTAAFEE EILWSQFLKF DKNRSKGIYQ LKVPPGYHPL VAILEREKQL QRIVTKLQME ADALLQSDVR AGEVERDLDK DVQTLKDGRH YRLHERLVAI GVAAPATQVA PELEDSTLRY NQHRVDGAEW LGSHRGLHQS ARSDEGQLSP GRLDLQYAKL LESLHSFVAA KEEEEVGFDW KESYSALMRE LQNAGDRLLR SFQAALQTQW EAHLKENAAY EGQLQKLQEA ATVTRLEDLL NEYKGHLSGL KPRHPAHPMR YKQVEVTVHK QPSHWKVLSS CFLVPPPNQE QHQALVTLWH AWQSLRRDVQ RTLKPEEQRQ AFLRDSQDAG EREYGSCSHH GAQEESRCQR QLEACETRTV PARECAQRIA GLGKGVARLS EPSPAAPTLR EQVRSLSAIY IRGTQGAEEV AQAVPATLPE LRAQAEAQQP GAQEVGERLQ ERWRERVAQL TDVRQRELEQ SADPLGAWLQ AMPLADSQAV LEEIERHGEK YINAIKDYEL PVASPAKKPK EYVDLRTHYS KFISETLRRM RAEERERLAE LAEAHAQAKA QRMQEEWRR | VAERDLGVTR QPDEKSIITY VPDVQDGVRA RELVLLLLQW RRFPSSFEEI KEMELPAKEA SLEGAVQAGQ DVEKEWGKLH RSEFERLECL AGLCEEQLNQ LLAAGKVPQR ADSMIRLLFN PQGEQMYRRV RTEYNLRLKA QVTLQSVQRR LQDLLAWVEE GVDLPSVEAQ IEEFRAKIER ATRGAYRDCL LNSSKARLRS ATKELMWLNE SDRNTNMTAK LELKEKKIKE EDHPARPTVE SWMLQLCCCI FQFFSDVREA LRRKYSCDRS QDAQDEKEQL AKRAKAVVQL GRLPLLAVCD GDECQLVGPA SGSEAAVPSV AQEAVTRLEA QLHVDMKSLL LIRSWSLATF ALHSLELHYQ GFGPEDRLMA YQQLLQSLEQ CISELKDIRL HRLRLPLDKE EQQKAQAEVE AEAEKVLALP SELELTLGKL LEKLKTISLV LRAHEEQLKE LEATKASLKK TFDALRDELR QRHGERDVEV LERWQAVLAQ LGRQLRYYRE DARRRQEQIQ REQLRQEQAL VEECQRFAKQ QLVTYKAQLE VQSGSESVIQ ELTTLTSQYI EEEERLAEQQ VEAALEKQRQ QAEREAKELQ EEAAVDAQQQ |
| | | | | | KRSIQEELQQ AKARQAEAAE RVVRLQLEAT LQALRARAEE EAERLRRQVQ VELASRVKAE LQALEELRLQ AEVERARQVQ EAELQSKRAS SLQEEHVAVA QQQAEAERAR QLKANEALRL KSLAQAEAEK RRGKAEEQAV EKQRQLAEGT LIRLRAETEQ ELARLQREAA AELAKVRAEM EEESRSTSEK RFRELAEEAA RQRQLAEEDA LAEKLAAIGE ALKEKEAENE FQRRRLEEQA LAQLRKASDS DTLRQRRQVE EKAAAGKAEL AEDTLRSKEQ LAAEEERRRR AAEEEAARQR KAKVEEARRL QLQLAQEAAQ AFAVQQKEQE VLDQLRGEAE EARVQAEREA ERLKQSAEEQ AEKLRKEAEQ AALRQKQAAD EQTLRQKAQV LEETDHQKNL EATEAARQRS VQMEELSKLK ILRDKDNTQR QVAEEAARLS QLAEEDLAQQ RALAEKMLKE QKELAQEQAR QQLAEETQGF QRTLEAERQR AQARAEEDAQ EKLHRTELAT EIQRQQSDHD LEREKEKLQQ EMQTVQQEQL FLSEKDSLLQ KLEQLFQDEV QRQQQQMEQE ARRRQHEAEE QLEQQRRQQE REQLQLLEEQ | LRQSSEAEIQ RSRLRIEEEI ERQRGGAEGE AEAQKRQAQE DESQRKRQAE AEAAREKQRA AEEAERRLRQ VALETAQRSA FAEKTAQLER QLREEAERRA EEAERELERW RLQAEEVAQQ QKEEAEREAR RQRELAEQEL AQQRLAAEQE GEQQRQLLEE AATQKRQELE EVLLASKARA SKQRLEAEAG RLRALAEEAK ARQRAEAERV ATRLKTEAEI RLRRLAEDEA AQHKADIEER ELERQKGLVE EEILALKASF ELELGRIRSN AELEAARQRQ EAEERVQKSL KAALEEVERL RERAEQESAR KRLQAEEKAH LQQTLQQEQS AARRAAEEAE AQSRRQVEEA AQARAQAQAA EAARRAQAEQ AEMEKHKKFA EQELTTLRLQ LDEELQRLKA QVEEELFSVR ARIEAENRAL FLQEEAEKMK VAAQEAARLR KMQAVQEATR LKAEAELLQQ RLQEDKEQMA QLEMSAEAER LKLRVAEMSR RFRKQAEEIG QEKVTLVQTL AERLREAIAE EAKLLQLKSE LQETQALQQS RERFIEQEKA AKAQQLREEQ RQRLVASMEE GVRRKQEELQ ELLAEENQRL HRAALAHSEE |
| | | | | | VTASQVAATK GPAAEAEPEH AQRLQEAGIL GHTTVDELAR RSSIAGLLLK ALQRQLLSPG ASGFLLDPVR KEGVVGPELH TGYKDPYTGQ GLIVREHGIR VIDPVHSHRV FDEEMNRVLA DPNTHENLTY PETGLCLLPL VYTDSEARDV GKFQGKTVTI AEQRRDLLRQ IIKIIITVVE EGLRSLVPAA ELYQQLQRGE TVRRALRGAN GQKLSIYNAL VALLEAQAGT RLTVDEAVRA LLSAEKAVTG SLFQALKKGL DAQLSTGGIV DVACARGCLD RADAKAYSDP LQQRCRPDQL KAARARQEEL EKTPVEVPVG ELISSEYFTA RTGKVTVEKV VETLRQERLS SELLASGVLS KTTVKDLSEL GCLAGIYLED MRRGLLRATT TGFLVDPVRN AGVVGPELHE GYRDPYSGST LVLRQHGIRL IDPVHSHRVP SEEMNRVLAD PNTHENLTYR ETGLRLLPLK TQVYTEEETR PGGGSHGGST LIPEEQRAQL KERMIIIIIE QGLASYDYVR ARIISLETYN EALEAESAWC VYLPGSRQTL LSAEVARLLL DPVKGERLTV PELHDRLLSA YTEQTISLFQ EALRLLDAQL | TLPNGRDALD SFDGLRRKVS SAEELQRLAQ REDVRHYLQG ATNEKLSVYA TALILLEAQA NRRLTVNEAV HKLLSAERAV QISLFQAMQK LLEAQIATGG PVDVAYRRGY DPSDDTKGFF LQLLERCVED TDKAAKGGEL FEKATVSAPF WEIINSEYFT FRTGRITVEK EQEQKGRLCF ELLESRVIDR RSVRDVAEVD VIAGVWLEEA KKDLLPSDMA GHIIDPATSA GLVGPEFHEK YRDPYTGQSV IPREQGLRLL DPSKSHRVPL EETSRALSAP STGEPATYGE TGLSLLPLSE YSELQARETF GFKGRTVTVW EQRQELLRQF IKILITIVEE FSGLRAPVPA RAQFEQLKDG GSVRTLLQGS TKEKVSIYEA AALLLEAQAA QRLYVHEAVK QLLSAEKAVT ISLFQAMQKG LEAQIATGGI VDVAYQRGYF PSDDTKGFFD QLLERCVEDP GAEKAEVVET RAFEETQIDI MSLWEVMQSD MADFQAGRVT IIEKTEIIRQ RRLTAEDLFE LLREGTRSLR YLYGTGSVAG SIYQALKKGL EAQAATGFLL DEAVRKGLVG ERAVTGYRDP AMKKELIPTE ATGGIVDPRL |
| | | | | | GFHLPLEVAY DQLSEPSEVR LSYTQLLRRC LPLSDARKLT EELVRSQVMD LTSIEEVTKN IAGVFVDATK KGIIRPGTAF YVIDPIKGLK IVGPEFKDKL KDPYSGKLIS LKDHGIRLLE PEESHRLPVE EMNEILTDPS TEENLTYLQL GLCLLPLKEK SVRKRRVVIV YEAYRKGLID ECEWEEITIS IDRRSGRQYD DRSALDQYRA MLSGNAGGFR SYPISPAVSR EETGPVAGIL TEAMHRNLVD QACTGGIIDP AVNKGLVDKI AFCGFEDPRT KKGWLYYEAG GGLIEPDTPG GTVDARTAQK LTCPKTKLKI VEEGTGLRLL YSPYSVSGSG TGSRAGSRRG MTFSSSSYSS SSASLGGPES | QRGYLNKDTH SYVDPSTDER RRDDGTGQLL FRGLRKQITM EATALQLREG LQKFLEGTSC ERLSVYQAMK ELLEAQAATG LTVEEAVRMG LSAERAVTGY LFQAMKKGLI AQIATGGIID VAYKRGLFDE DDTKGFFDPN MERCITDPQT KRERKTSSKS DPETGKEMSV HQTYLELSEQ SSDGVVKSMI IDDAIAKNLI GTLSITEFAD SRSSSVGSSS TQLASWSDPT DTETLEKVSI NITGQRLLEA STGERFPVTD MVDRINLAQK KTKMSAAQAL QRFLEVQYLT RVPLDEALQR LRDVGAYSKY SYKDALDRSM EAAAQSTKGY STAGSRTGSR SFDATGSGFS SGYGRRYASG AVA |
| 76 | ACSLI | 1 | P33121 | Entry version 186 (18 Sep. 2019) Sequence version 1 (01 Oct. 1993) | MQAHELFRYF YVRTLPTNTL LKPPCDLSMQ SVEVAGSGGA PLVYFYDDVT QVSNNGPCLG LSYKQVAELS GFKTAPDQFI VIIEQGCFAY LGNEAITYIV DKPEKAKLLL LKIIVVMDAY CGVEVTSMKA KPKPPAPEDL GNPKGAMVTH VKATENTVNP PLAHMFERVV IGFFQGDIRL TVFPVVPRLL ANTTLKRWLL LRSGIIRNNS QSSLGGRVRL TVLTFLRAAL | RMPELVDFRQ MGFGAFAALT TFWYATRPKP RRSALLDSDE TLYEGFQRGI SRKPDQPYEW ECIGSALIQK GIFAQNRPEW SMVIVPLYDT NKAELSLVFV EGVENKLIPG GSELVERGQR MEDLGRANRR AVICFTSGTT RNIVSDCSAF CPDDTLISFL ECVMLCHGAK LMDDLKVLQP NRMFDRIFGQ DFASKRKEAE LWDRLIFHKV MVTGAAPVSA GCQFYEGYGQ |
| | | | | | TECTAGCCLT GAPMPCNLIK AAEGEGEVCV KDPAKTAEAL IGKWLPNGTL KLAQGEYIAP EPVAQVFVHG VVPDVETLCS FEELCRNKDV LGKDSGLKPF ELFSIDNGLL LRNYFRSQID | MPGDWTAGHV LVDVEEMNYM KGPNVFQGYL DKDGWLHTGD KIIDRKKHIF EKIENIYMRS ESLQAFLIAI WAQKRGFEGS KKAILEDMVR EQVKGITLHP TPTMKAKRPE DLYSTIK |
| 77 | ACSLI | 2 | P331212 | Entry version186 (18 Sep. 2019) Sequence version 1 (01 Oct. 1993) | MQAHELFRYF YVRTLPTNTL TFWYATRPKP SVEVAGSGGA PLVYFYDDVT QVSNNGPCLG LSYKQVAELS GFKTAPDQFI VIIEQGCFAY LGNEAITYIV DKPEKAKLLL LKIIVVMDAY CGVEVTSMKA KPKPPAPEDL GNPKGAMVTH VKATENTVNP PLAHMFERVV IGFFQGDIRL TVFPVVPRLL ANTTLKRWLL LRSGIIRNNS QSSLGGRVRL TVLTFLRAAL TECTAGCCLT GAPMPCNLIK AAEGEGEGYL DKDGWLHTGD KIIDRKKHIF EKIENIYMRS ESLQAFLIAI WAQKRGFEGS KKAILEDMVR EQVKGITLHP TPTMKAKRPE DLYSTIKV | RMPELVDFRQ MGFGAFAALT LKPPCDLSMQ RRSALLDSDE TLYEGFQRGI SRKPDQPYEW ECIGSALIQK GIFAQNRPEW SMVIVPLYDT NKAELSLVFV EGVENKLIPG GSELVERGQR MEDLGRANRR AVICFTSGTT RNIVSDCSAF CPDDTLISFL ECVMLCHGAK LMDDLKVLQP NRMFDRIFGQ DFASKRKEAE LWDRLIFHKV MVTGAAPVSA GCQFYEGYGQ MPGDWTAGHV LVDVEEMNYM KDPAKTAEAL IGKWLPNGTL KLAQGEYIAP EPVAQVFVHG VVPDVETLCS FEELCRNKDV LGKDSGLKPF ELFSIDNGLL LRNYFRSQID |
| 78 | ACSLI | 3 | P331213 | Entry version 186 (18 Sep. 2019) Sequence version 1 (01 Oct. 1993) | MQAHELFRYF YVRTLPTNTL TFWYATRPKP SVEVAGSGGA PLVYFYDDVT QVSNNGPCLG LSYKQVAELS GFKTAPDQFI VIIEQGCFAY LGNEAITYIV DKPEKAKLLL LKIIVVMDAY CGVEVTSMKA | RMPELVDFRQ MGFGAFAALT LKPPCDLSMQ RRSALLDSDE TLYEGFQRGI SRKPDQPYEW ECIGSALIQK GIFAQNRPEW SMVIVPLYDT NKAELSLVFV EGVENKLIPG GSELVERGQR MEDLGRANRR |
| | | | | | KPKPPAPEDL GNPKGAMVTH VKATEKALPL PLAHIYEQLL IGFFQGDIRL TVFPVVPRLL ANTTLKRWLL LRSGIIRNNS QSSLGGRVRL TVLTFLRAAL TECTAGCCLT GAPMPCNLIK AAEGEGEVCV KDPAKTAEAL IGKWLPNGTL KLAQGEYIAP EPVAQVFVHG VVPDVETLCS FEELCRNKDV LGKDSGLKPF ELFSIDNGLL LRNYFRSQID | AVICFTSGTT RNIVSDCSAF SASDTHISYL KCVMLCHGAK LMDDLKVLQP NRMFDRIFGQ DFASKRKEAE LWDRLIFHKV MVTGAAPVSA GCQFYEGYGQ MPGDWTAGHV LVDVEEMNYM KGPNVFQGYL DKDGWLHTGD KIIDRKKHIF EKIENIYMRS ESLQAFLIAI WAQKRGFEGS KKAILEDMVR EQVKGITLHP TPTMKAKRPE DLYSTIKV |
| 79 | RAC1 | 1 | P63000 | Entry version 192 (18 Sep. 2019) Sequence version 1 (31 Aug. 2004) | MQAIKCVVVG ISYTTNAFPG SANVMVDGKP QEDYDRLRPL CFSLVSPASF VRHHCPNTPI DDKDTIEKLK QGLAMAKEIG TQRGLKTVFD PVKKRKRKCL | DGAVGKTCLL EYIPTVFDNY VNLGLWDTAG SYPQTDVFLI ENVRAKWYPE ILVGTKLDLR EKKLTPITYP AVKYLECSAL EAIRAVLCPP LL |
| 80 | RAC1 | 2 | P630002 | Entry version 192 (18 Sep. 2019) Sequence version 1 (31 Aug. 2004) | MQAIKCVVVG ISYTTNAFPG SANVMVDGKP QEDYDRLRPL GKDITSRGKD FSLVSPASFE RHHCPNTPII DKDTIEKLKE GLAMAKEIGA QRGLKTVFDE VKKRKRKCLL | DGAVGKTCLL EYIPTVFDNY VNLGLWDTAG SYPQTVGETY KPIADVFLIC NVRAKWYPEV LVGTKLDLRD KKLTPITYPQ VKYLECSALT AIRAVLCPPP L |
| 81 | PSMB2 | 1 | P49721 | Entry version 201 (18 Sep. 2019) Sequence version 1 (01 Oct. 1996) | MEYLIGIQGP AASNIVQMKD KILLLCVGEA QKNVQLYKMR ANFTRRNLAD NLLLAGYDEH LAALAKAPFA SILDRYYTPT RKCLEELQKR RIIDKNGIHD S | DYVLVASDRV DHDKMFKMSE GDTVQFAEYI NGYELSPTAA CLRSRTPYHV EGPALYYMDY AHGYGAFLTL ISRERAVELL FILNLPTFSV LDNISFPKQG |
| 82 | GM2A | 1 | P17900 | Entry version 190 (18 Sep. 2019) | MQSLMQAPLL AQAHLKKPSQ EGKDPAVIRS | IALGLLLAAP LSSFSWDNCD LTLEPDPIIV |
| | | | | Sequence version 4 (13 Nov. 2007 | PGNVTLSVMG KVDLVLEKEV DYIGSCTFEH TGEPCPEPLR KEGTYSLPKS SWLTTGNYRI LGCIKIAASL | STSVPLSSPL AGLWIKIPCT FCDVLDMLIP TYGLPCHCPF EFVVPDLELP ESVLSSSGKR KGI |

### VARIANT GENE SEQUENCES

| SEQ ID NO. | GENE | Accession No. | Ensembl Release No. |
|---|---|---|---|
| 83 | CAP37 | ENSG00000278624 | 98 |
| 84 | TAPBP | ENSG00000112493 | 98 |
| 85 | TAPBP | ENSG00000206281 | 98 |
| 86 | TAPBP | ENSG00000112493 | 98 |
| 87 | TAPBP | ENSG00000236490 | 98 |

### EXAMPLES

### Materials and Methods

### Isolation of Stem Cell-Derived Neutrophils

Stem cell-derived neutrophils (SCDN) were synthesised according to standard techniques and cultured ex vivo for 25 days following Ficoll-separation to obtain PBMCs and CD34+ isolates from ten one-off donor buffy leukocyte cones. Aliquots of the SCDN (50 × 10⁶ /ml) were frozen at -80° C. in cryopreservative (10% FBS in DMSO).

### Evaluation of Cancer Killing Activity (CKA) Using the xCelligence Assay

SCDN were thawed and decanted into complete Dulbecco’s modified Eagle’s medium (DMEM) before incubation for 72 hours with pancreatic cancer (PANC-1) cells and ‘healthy’ breast epithelial cells (MCF-12F) (commercially available from the American Type Culture Collection - United Kingdom (U.K.), Guernsey, Ireland, Jersey and Liechtenstein, LGC Standards, Queens Road, Teddington, Middlesex TW11 0LY, UK). CKA was recorded regularly throughout the 72 hour culture period by xCelligence Assay.

The ACEA Biosciences xCELLigence RTCA DP Analyzer system® was used and the manufacturer’s instructions were followed. The xCELLigence System is a real-time cell analyser, allowing for label-free and dynamic monitoring of cellular phenotypic changes continuously by measuring electrical impedance. The system measures impedance using interdigitated gold microelectrodes integrated into the bottom of each well of the tissue culture E-Plates. Impedance measurements are displayed as Cell Index (CI) values, providing quantitative information about the biological status of the cells, including viability. Impedance-based monitoring of cell viability correlates with cell number and MTT-based readout. The kinetic aspect of impedance-based cell viability measurements provides the necessary temporal information when neutrophils are used to induce cytotoxic effects. In particular, the xCELLigence System can also pinpoint the optimal time points when the neutrophils achieve their maximal effect (where such data is desired), as indicated by the lowest CI values, in cytotoxicity and cell death assays. 6,000 cancer cells (PANC-1) or healthy, non-cancerous cells (MCF-12F) are placed in the bottom of a 16 well plate (the system can read up to 3 plates simultaneously). For the first few hours after cells have been added to a well there is a rapid increase in impedance. This is caused by cells falling out of suspension, depositing onto the electrodes, and forming focal adhesions. If the initial number of cells added is low and there is empty space on the well bottom, cells will proliferate, causing a gradual yet steady increase in Cl. When cells reach confluence the CI value plateaus, reflecting the fact that the electrode surface area that is accessible to bulk media is no longer changing. At this point, which is called the ‘normalization point’, the neutrophils (60,000 cells) are added (giving a 10:1 effector:target ratio) and incubated at 37° C. The percentage of cytolysis is readily calculated using a simple formula: Percentage of cytolysis = ((Cell indexₙₒ_{effector} - Cell Index_{effector})/Cell Indexₙₒ_{effector}) × 100.

SCDNs that demonstrated >80% CKA (against PANC-1) by 48 hours after addition, and <10% off-target killing (i.e. killed <10% of ‘healthy’ breast epithelial cells (MCF-12F) by 48 hours after addition) were designated high CKA neutrophils and cells that demonstrated <51.5% CKA were designated low CKA control neutrophils.

### Proteomic Analysis

Neutrophils were lysed and underwent sonication and were analysed using the Pierce bicinchoninic acid (BCA) protein assay according to manufacturer’s instructions (commercially available from ThermoFisher, Waltham, MA, catalogue number: 23225) to determine protein concentration. Typically samples contained around 20 micrograms of protein in <500 µl. Samples were digested, desalted and lyophilised prior to liquid chromatography and mass spectrometry (LC-MS/MS) using a Thermo Q-Exactive (Orbitrap) Plus Mass Spectrometer (Thermo Scientific™). First, chromatography separates the peptides in solution, the smaller hydrophilic peptides come off the column in the first fraction, and bigger hydrophobic peptides come off last over a 2 hour period. Secondly, a strongly acidic pH2 solution ensures all peptides have protons and are thus given a positive charge, the Mass Spectrometer only allows through positively charged ions of a given fraction to hit the detector. The Orbitrap device fluctuates between isolate and fragment, at around 20 Hz so the least ‘sticky’ peptides of a given mass/charge ratio are quantified first. The fluctuations are proportional to the intensity of the peptides detected, thus providing protein quantities for each cell type.

Bioinformatics was performed using the online DAVID system (Huang DW, Sherman BT, Lempicki RA. Bioinformatics enrichment tools: paths toward the comprehensive functional analysis of large gene lists. Nucleic Acids Res. 2009;37:1-13.; and Huang DW, Sherman BT, Lempicki RA. Systematic and integrative analysis of large gene lists using DAVID bioinformatics resources. Nat Protoc. 2009;4:44-57).

### Example 1

Proteomic analysis was carried out according to the Materials & Methods section herein. Advantageously, a number of proteins were significantly upregulated in the high CKA neutrophils when compared to low CKA controls.

The following proteins (and thus genes) were upregulated compared to low CKA controls: S100A9, S100A8, ITGB1, CYBB, SYK, DOCK8, COMP, ATG7, SLC2A1, GZMK, CTSG, ATM, IKBKB, BCAP31, TAPBP, PERM, PLEC, ACSL1, RAC1, GM2A, CAP37, and PSMB2.

The following proteins (and thus genes) were downregulated compared to low CKA controls: ANXA1 and PPP3CB.

Table 1 presents a number of proteins with changed expression in high CKA cells compared to the typical low CKA cells.

| Protein | log2 (High CKA/Typical) | p-value (t-test) |
|---|---|---|
| GM2A | 1.943649 | 7.66E-06 |
| PLEC | 0.855651 | 0.000199 |
| CYBB | 1.609576 | 0.00024 |
| DOCK8 | 1.454172 | 0.000455 |
| ATG7 | 1.163505 | 0.000737 |
| SLC2A1 | 1.4505 | 0.001259 |
| S100A9 | 1.435349 | 0.001681 |
| ACSL1 | 1.065324 | 0.001746 |
| CTSG | 2.155855 | 0.002295 |
| PSMB2 | 1.058384 | 0.002481 |
| ATM | 2.011409 | 0.002754 |
| BCAP31 | 2.707073 | 0.003322 |
| S100A8 | 0.699763 | 0.003776 |
| ITGB1 | 1.143777 | 0.005138 |
| TAPBP | 1.277103 | 0.005596 |
| COMP | 0.99287 | 0.005695 |
| SYK | 1.722188 | 0.006851 |
| GZMK | 1.8696069 | 0.040591 |
| IKBKB | 2.2958852 | 0.017181 |
| PPP3CB | -1.36037 | 0.000127 |
| ANXA1 | -1.17725 | 0.000792 |
| PERM | 2.00962408 | 0.0379030 |
| RAC1 | 1.9456240 | 0.0100812 |
| CAP37 | 2.70390702 | 0.01459156 |

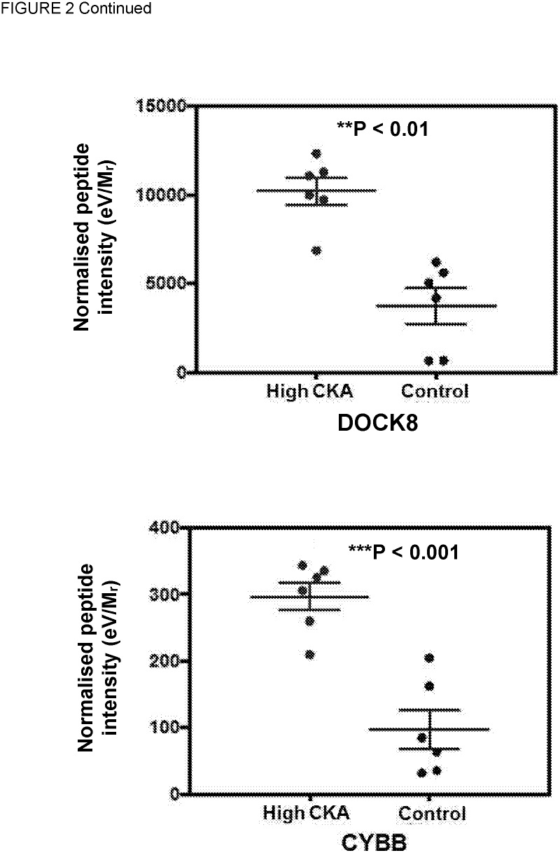

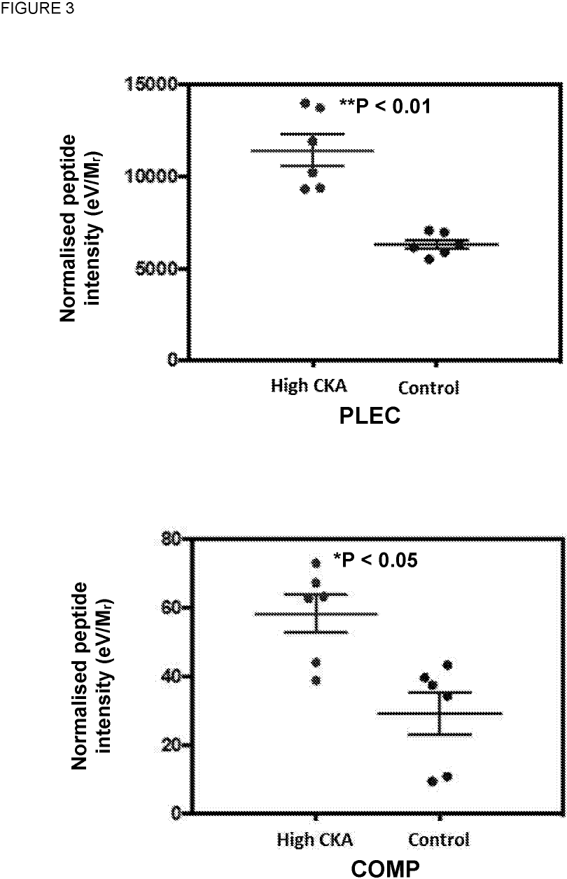

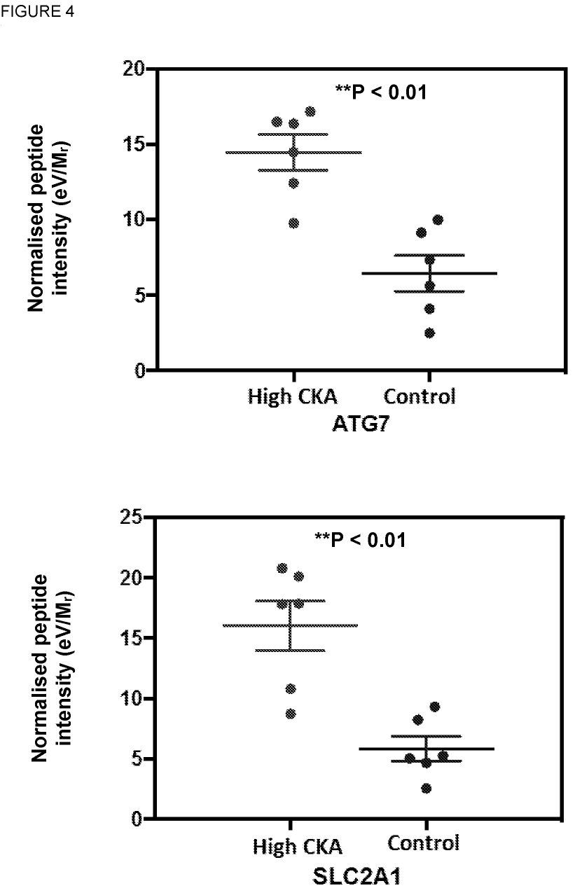

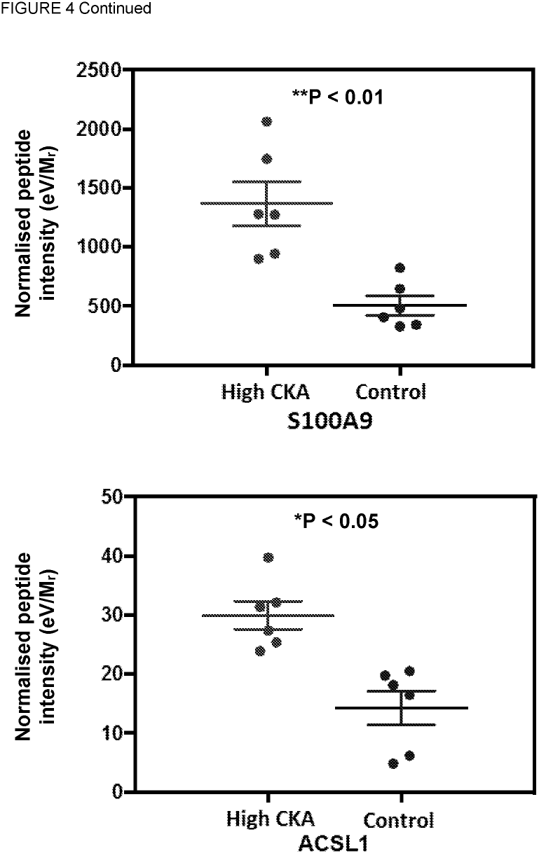

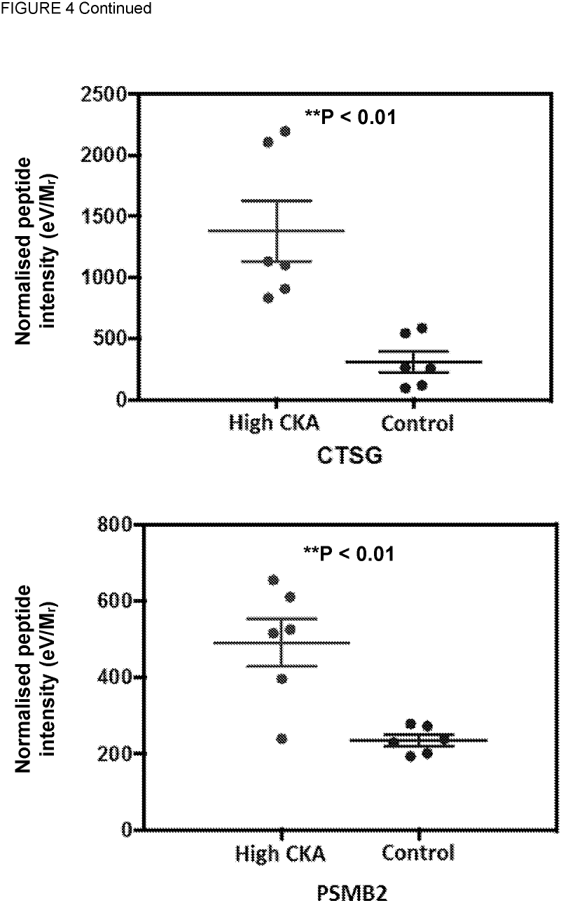

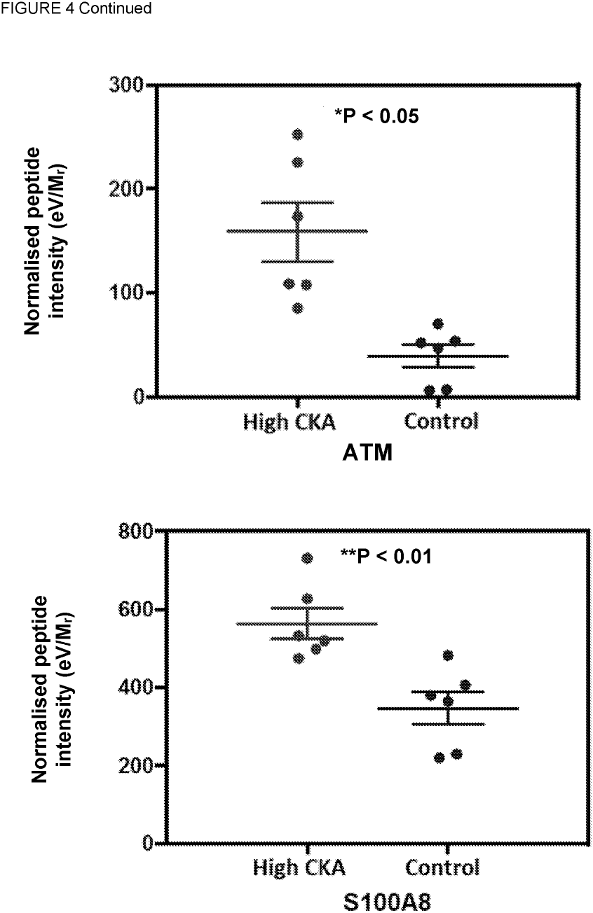

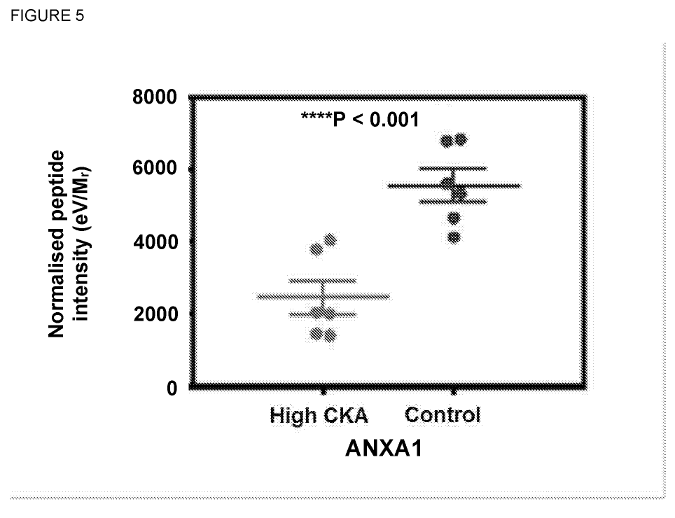

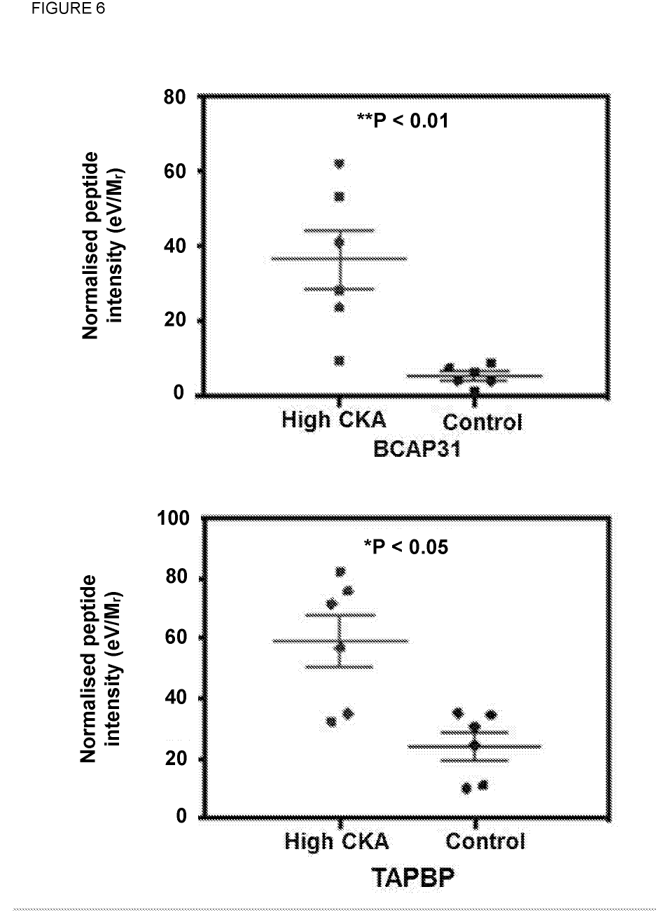

The results are presented graphically in FIGS. 2-6 for a number of the proteins, including two-way ANOVA statistical analyses. Advantageously, said protein levels (and thus gene expression levels) provided a robust means for identifying and differentiating high CKA cells from low CKA cells.

Advantageously, the expression of many of the genes (i.e. at the protein level) was highly statistically-significantly different (e.g. GM2A) between high CKA cells and low CKA cells, indicating that high CKA granulocytes could be identified using just one of the indicated genes.

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the following claims.

1. A method for determining the suitability of a granulocyte for treating cancer, the method comprising:
a. comparing a measured expression level of one or more genes by the granulocyte, wherein the one or more genes are associated with suitability for treating cancer and are selected from: GM2A, CTSG, CAP37, ITGB1, CYBB, SYK, DOCK8, COMP, ATG7, SLC2A1, GZMK, ATM, IKBKB, BCAP31, TAPBP, PPP3CB, ANXA1, PERM, PLEC, ACSL1, RAC1, and PSMB2, with the expression level of the same one or more genes in a reference standard; and
b. determining the suitability of the granulocyte for treating cancer based on the comparison.

2. A method for identifying whether or not a donor produces granulocytes suitable for treating cancer, the method comprising:
a. comparing a measured expression level of one or more genes by a granulocyte comprised in a sample obtainable from the donor, wherein the one or more genes are associated with suitability for treating cancer and are selected from: GM2A, CTSG, CAP37, ITGB1, CYBB, SYK, DOCK8,COMP, ATG7, SLC2A1, GZMK, ATM, IKBKB, BCAP31, TAPBP, PPP3CB, ANXA1, PERM, PLEC, ACSL1, RAC1, and PSMB2, with the expression level of the same one or more genes in a reference standard; and
b. identifying whether or not the donor produces granulocytes suitable for treating cancer based on the comparison.

3. The method according to claim 1 or 2, wherein step b. comprises:
determining that the granulocyte is suitable for treating cancer or identifying that the donor produces granulocytes suitable for treating cancer when:
i. the measured expression level of one or more of GM2A, CTSG, CAP37, ITGB1, CYBB, SYK, DOCK8,COMP, ATG7, SLC2A1, GZMK, ATM, IKBKB, BCAP31, TAPBP, PERM, PLEC, ACSL1, RAC1, and PSMB2 is increased when compared to the reference standard when the reference standard is from a granulocyte unsuitable for treating cancer; or
ii. the measured expression level of ANXA1 and/or PPP3CB is decreased when compared to the reference standard, when the reference standard is from a granulocyte unsuitable for treating cancer; or
iii. the measured expression level of one or more of GM2A, CTSG, CAP37, ITGB1, CYBB, SYK, DOCK8,COMP, ATG7, SLC2A1, GZMK, ATM, IKBKB, BCAP31, TAPBP, PERM, PLEC, ACSL1, RAC1, and PSMB2 is increased or the same when compared to the reference standard, when the reference standard is from a granulocyte suitable for treating cancer; or
iv. the measured expression level of ANXA1 and/or PPP3CB is decreased or the same when compared to the reference standard, when the reference standard is from a granulocyte suitable for treating cancer; or
determining that the granulocyte is unsuitable for treating cancer or identifying that the donor produces granulocytes that are unsuitable for treating cancer when:
v. the measured expression level of one or more of GM2A, CTSG, CAP37, ITGB1, CYBB, SYK, DOCK8,COMP, ATG7, SLC2A1, GZMK, ATM, IKBKB, BCAP31, TAPBP, PERM, PLEC, ACSL1, RAC1, and PSMB2 is decreased or the same when compared to the reference standard, when the reference standard is from a granulocyte unsuitable for treating cancer; or
vi. the measured expression level of ANXA1 and/or PPP3CB is increased or the same when compared to the reference standard, when the reference standard is from a granulocyte unsuitable for treating cancer; or
vii. the measured expression level of one or more of GM2A, CTSG, CAP37, ITGB1, CYBB, SYK, DOCK8,COMP, ATG7, SLC2A1, GZMK, ATM, IKBKB, BCAP31, TAPBP, PERM, PLEC, ACSL1, RAC1, and PSMB2 is decreased when compared to reference standard, when the reference standard is from a granulocyte suitable for treating cancer; or
viii. the measured expression level of ANXA1 and/or PPP3CB is increased when compared to the reference standard, when the reference standard is from a granulocyte suitable for treating cancer.

4. A method for selecting whether or not a subject is suitable for treatment with a granulocyte or a stem cell for treating cancer, the method comprising:
a. comparing a measured expression level of one or more genes by a granulocyte comprised in a sample obtainable from the subject, wherein the one or more genes are associated with suitability for treating cancer and are selected from: GM2A, CTSG, CAP37, ITGB1, CYBB, SYK, DOCK8,COMP, ATG7, SLC2A1, GZMK, ATM, IKBKB, BCAP31, TAPBP, PPP3CB, ANXA1, PERM, PLEC, ACSL1, RAC1, and PSMB2with the expression level of the same one or more genes in a reference standard; and
b. identifying whether or not the subject is suitable for treatment with a granulocyte or a stem cell for treating cancer based on the comparison.

5. A method for determining a subject’s risk for developing cancer, the method comprising:
a. comparing a measured expression level of one or more genes by a granulocyte comprised in a sample obtainable from the subject, wherein the one or more genes are associated with suitability for treating cancer and are selected from: GM2A, CTSG, CAP37, ITGB1, CYBB, SYK, DOCK8,COMP, ATG7, SLC2A1, GZMK, ATM, IKBKB, BCAP31, TAPBP, PPP3CB, ANXA1, PERM, PLEC, ACSL1, RAC1, and PSMB2with the expression level of the same one or more genes in a reference standard; and
b. determining the subject’s risk for developing cancer based on the comparison.

6. The method according to claim 4 or 5, wherein step b. comprises:
identifying the subject as suitable for treatment with a granulocyte or a stem cell for treating cancer or determining that the subject is at risk of developing cancer when:
i. the measured expression level of one or more of GM2A, CTSG, CAP37, ITGB1, CYBB, SYK, DOCK8,COMP, ATG7, SLC2A1, GZMKATM, IKBKB, BCAP31, TAPBP, PERM, PLEC, ACSL1, RAC1, and PSMB2 is decreased or the same when compared to the reference standard, when the reference standard is from a granulocyte unsuitable for treating cancer; or
ii. the measured expression level of ANXA1 and/or PPP3CB is increased or the same when compared to the reference standard, when the reference standard is from a granulocyte unsuitable for treating cancer; or
iii. the measured expression level of one or more of GM2A, CTSG, CAP37, ITGB1, CYBB, SYK, DOCK8,COMP, ATG7, SLC2A1, GZMK, ATM, IKBKB, BCAP31, TAPBP, PERM, PLEC, ACSL1, RAC1, and PSMB2 is decreased when compared to the reference standard, when the reference standard is from a granulocyte suitable for treating cancer; or
iv. the measured expression level of ANXA1 and/or PPP3CB is increased when compared to the reference standard, when the reference standard is from a granulocyte suitable for treating cancer; or
identifying the subject as unsuitable for treatment with a granulocyte or a stem cell for treating cancer when or determining that the subject is not at risk of developing cancer when:
v. the measured expression level of one or more of GM2A, CTSG, CAP37, ITGB1, CYBB, SYK, DOCK8,COMP, ATG7, SLC2A1, GZMK, ATM, IKBKB, BCAP31, TAPBP, PERM, PLEC, ACSL1, RAC1, and PSMB2 is increased when compared to the reference standard when the reference standard is from a granulocyte unsuitable for treating cancer; or
vi. the measured expression level of ANXA1 and/or PPP3CB is decreased when compared to the reference standard, when the reference standard is from a granulocyte unsuitable for treating cancer; or
vii. the measured expression level of one or more of GM2A, CTSG, CAP37, ITGB1, CYBB, SYK, DOCK8,COMP, ATG7, SLC2A1, GZMK, ATM, IKBKB, BCAP31, TAPBP, PERM, PLEC, ACSL1, RAC1, and PSMB2 is increased or the same when compared to the reference standard, when the reference standard is from a granulocyte suitable for treating cancer; or
viii. the measured expression level of ANXA1 and/or PPP3CB is decreased or the same when compared to the reference standard, when the reference standard is from a granulocyte suitable for treating cancer.

7. The method according any one of the preceding claims, wherein the method further comprises measuring the expression of the one or more genes.

8. The method according to any one of the preceding claims, wherein the granulocyte is a neutrophil.

9. The method according to any one of the preceding claims, further comprising measuring the expression level of one or more gene selected from: S100A9 and S100A8.

10. The method according to any one of the preceding claims, wherein the one or more genes associated with suitability for treating cancer are one or more genes associated with:
a. killing cancer cells and are selected from: GM2A, CTSG, CAP37, CYBB, GZMK, ATM, PERM, ACSL1, ATG7, SYK, DOCK8, RAC1, and PSMB2; and/or
b. locating and/or binding to cancer cells and are selected from: ANXA1, ITGB1, COMP, SLC2A1 and PLEC; and/or
c. recruitment of immune mediators and are selected from: BCAP31, TAPBP, IKBKB, and PPP3CB.

11. The method according to any one of the preceding claims, wherein the expression level is measured by proteomic techniques.

12. The method according to any one of the preceding claims, wherein the expression level is measured by transcriptomic techniques.

13. The method according to any one of claims 1-3 or 7-12 further comprising selecting a granulocyte when the granulocyte has been determined to be suitable for treating cancer or selecting a granulocyte from a sample obtainable from a donor when the donor has been identified as a donor that produces granulocytes for treating cancer, preferably wherein the selecting comprises isolating the granulocyte.

14. An in vitro method for obtaining a granulocyte for treating cancer, said method comprising obtaining a granulocyte from a sample obtainable from a donor wherein said donor produces granulocytes comprising:
a. increased expression of one or more of ITGB1, CYBB, SYK, DOCK8, COMP, ATG7, SLC2A1, GZMK, CTSG, ATM, IKBKB, BCAP31, TAPBP, PERM, PLEC, ACSL1, RAC1, GM2A, CAP37, and PSMB2 when compared to a reference standard, wherein the reference standard is from a granulocyte unsuitable for treating cancer; and/or
b. decreased expression of ANXA1 and/or PPP3CB when compared to a reference standard, wherein the reference standard is from a granulocyte unsuitable for treating cancer.

15. An in vitro method for obtaining a stem cell for treating cancer, said method comprising obtaining a stem cell from a sample obtainable from a donor wherein said donor produces granulocytes comprising:
a. increased expression of one or more of ITGB1, CYBB, SYK, DOCK8, COMP, ATG7, SLC2A1, GZMK, CTSG, ATM, IKBKB, BCAP31, TAPBP, PERM, PLEC, ACSL1, RAC1, GM2A, CAP37, and PSMB2 when compared to a reference standard, wherein the reference standard is from a granulocyte unsuitable for treating cancer; and/or
b. decreased expression of ANXA1 and/or PPP3CB when compared to a reference standard, wherein the reference standard is from a granulocyte unsuitable for treating cancer.

16. A method for preparing a stem cell suitable for treating cancer, the method comprising:
a. identifying a donor that produces granulocytes suitable for treating cancer according to the method of any one of claims 2-3 or 7-13; and
b. obtaining the stem cell from a sample obtainable from said donor.

17. A method for producing a stem cell for treating cancer, the method comprising:
a. providing a stem cell obtainable from a sample from a donor wherein said donor produces granulocytes comprising:
i. increased expression of one or more of ITGB1, CYBB, SYK, DOCK8, COMP, ATG7, SLC2A1, GZMK, CTSG, ATM, IKBKB, BCAP31, TAPBP, PERM, PLEC, ACSL1, RAC1, GM2A, CAP37, and PSMB2 when compared to a reference standard, wherein the reference standard is from a granulocyte unsuitable for treating cancer; and/or
ii. decreased expression of ANXA1 and/or PPP3CB when compared to a reference standard, wherein the reference standard is from a granulocyte unsuitable for treating cancer ; and
b. differentiating the stem cell into a different stem cell (preferably a precursor cell); and
c. optionally isolating the different stem cell (preferably the precursor cell).

18. The method according to any one of claims 15-17 wherein the sample comprises a somatic cell and obtaining the stem cell from the sample comprises reprograming the somatic cell into a stem cell.

19. A method for isolating a granulocyte or stem cell for treating cancer, the method comprising:
a. contacting a sample of granulocytes or a sample of stem cells with a binding means, wherein the binding means binds to one or more polypeptides selected from GM2A, CTSG, CAP37, ITGB1, CYBB, SYK, DOCK8,COMP, ATG7, SLC2A1, GZMK, ATM, IKBKB, BCAP31, TAPBP, PPP3CB, ANXA1, PERM, PLEC, ACSL1, RAC1, and PSMB2; and
b. isolating the granulocyte or stem cell, respectively, based on the presence or absence of binding between the binding means and the one or more polypeptides.

20. A method for producing an engineered granulocyte or stem cell for treating cancer, the method comprising:
a. providing a granulocyte or stem cell, respectively; and
b. engineering the granulocyte or stem cell, respectively, to:
i. increase expression of one or more genes selected from: GM2A, CTSG, CAP37, ITGB1, CYBB, SYK, DOCK8,COMP, ATG7, SLC2A1, GZMK, ATM, IKBKB, BCAP31, TAPBP, PERM, PLEC, ACSL1, RAC1, and PSMB2; and/or
ii. decrease expression of ANXA1 and/or PPP3CB;
thereby producing the engineered granulocyte or stem cell, respectively, wherein the
engineered granulocyte or stem cell, respectively, is suitable for treating cancer.

21. A stem cell for treating cancer obtainable by the method according to any one of claims 15-20.

22. A stem cell which is capable of differentiating into a granulocyte for treating cancer, wherein the granulocyte comprises:
a. increased expression of one or more of ITGB1, CYBB, SYK, DOCK8, COMP, ATG7, SLC2A1, GZMK, CTSG, ATM, IKBKB, BCAP31, TAPBP, PERM, PLEC, ACSL1, RAC1, GM2A, CAP37, and PSMB2 when compared to a reference standard, wherein the reference standard is from a granulocyte unsuitable for treating cancer; and/or
b. decreased expression of ANXA1 and/or PPP3CB when compared to a reference standard, wherein the reference standard is from a granulocyte unsuitable for treating cancer.

23. The stem cell according to claim 21 or 22, wherein the stem cell is an induced pluripotent stem cell, or wherein the stem cell is a haematopoietic stem cell, a common myeloid progenitor cell, a myeloblast, a N. promyelocyte, a N. myelocyte, a N. metamyelocyte, a N. band, or combinations thereof; preferably wherein the stem cell is a haematopoietic stem cell.

24. A method for producing a granulocyte for treating cancer, the method comprising:
a. providing a cell; and
b. converting the cell into a granulocyte wherein:
i. the measured expression level of one or more of GM2A, CTSG, CAP37, ITGB1, CYBB, SYK, DOCK8,COMP, ATG7, SLC2A1, GZMK, ATM, IKBKB, BCAP31, TAPBP, PERM, PLEC, ACSL1, RAC1, and PSMB2 is increased in the granulocyte when compared to a reference standard when the reference standard is from a granulocyte unsuitable for treating cancer; or
ii. the measured expression level of ANXA1 and/or PPP3CB is decreased in the granucloyte when compared to the reference standard, when the reference standard is from a granulocyte unsuitable for treating cancer; or
iii. the measured expression level of one or more of GM2A, CTSG, CAP37, ITGB1, CYBB, SYK, DOCK8,COMP, ATG7, SLC2A1, GZMK, ATM, IKBKB, BCAP31, TAPBP, PERM, PLEC, ACSL1, RAC1, and PSMB2 is increased or the same when compared to the reference standard, when the reference standard is from a granulocyte suitable for treating cancer; or
iv. the measured expression level of ANXA1 and/or PPP3CB is decreased or the same when compared to the reference standard, when the reference standard is from a granulocyte suitable for treating cancer; and
optionally isolating the granulocyte.

25. The method according to claim 24 wherein the cell is a stem cell according to any one of claims 21-23.

26. The method according to claim 25 wherein converting the stem cell into a granulocyte comprises differentiating the stem cell into a granulocyte.

27. The method according to claim 24 wherein the cell is a somatic/differentiated cell, optionally from a donor who produces granulocytes suitable for treating cancer as determined according to any one of claims 2-3 or 7-13.

28. The method according to claim 27 wherein converting the somatic/differentiated cell into a granulocyte comprises transdifferentiating the somatic/differentiated cell into a granulocyte.

29. A granulocyte (e.g. obtainable by the method according to any one of claims 13, 19-20 or 24-28), wherein the granulocyte comprises:
a. increased expression of one or more of GM2A, CTSG, CAP37, ITGB1, CYBB, SYK, DOCK8,COMP, ATG7, SLC2A1, GZMK, ATM, IKBKB, BCAP31, TAPBP, PERM, PLEC, ACSL1, RAC1, and PSMB2 when compared to a reference standard, wherein the reference standard is from a granulocyte unsuitable for treating cancer; and/or
b. decreased expression of ANXA1 and/or PPP3CB when compared to a reference standard, wherein the reference standard is from a granulocyte unsuitable for treating cancer.

30. A composition for treating cancer (e.g. obtainable by the method according to any one of claims 13, 19-20 or 24-28), the composition comprising granulocytes: wherein at least 90% of the granulocytes comprised in the composition have:
a. increased expression of one or more of GM2A, CTSG, CAP37, ITGB1, CYBB, SYK, DOCK8,COMP, ATG7, SLC2A1, GZMK, ATM, IKBKB, BCAP31, TAPBP, PERM, PLEC, ACSL1, RAC1, and PSMB2 when compared to a reference standard, wherein the reference standard is from a granulocyte unsuitable for treating cancer; and/or
b. decreased expression of ANXA1 and/or PPP3CB when compared to a reference standard, wherein the reference standard is from a granulocyte unsuitable for treating cancer.

31. The composition according to claim 30, wherein at least 95%, 99% or 100% of the granulocytes comprised in the composition have:
a. increased expression of one or more of GM2A, CTSG, CAP37, ITGB1, CYBB, SYK, DOCK8,COMP, ATG7, SLC2A1, GZMK, ATM, IKBKB, BCAP31, TAPBP, PERM, PLEC, ACSL1, RAC1, and PSMB2 when compared to a reference standard, wherein the reference standard is from a granulocyte unsuitable for treating cancer; and/or
b. decreased expression of ANXA1 and/or PPP3CB when compared to a reference standard, wherein the reference standard is from a granulocyte unsuitable for treating cancer.

32. A pharmaceutical composition comprising:
a. the granulocyte according to claim 29, the stem cell according to any one of claims 21-23 , or the composition according to claim 30 or 31; and
b. a pharmaceutically acceptable carrier, excipient, adjuvant, and/or salt.

33. The pharmaceutical composition according to claim 32, further comprising TNF-alpha.

34. The pharmaceutical composition according to any one of claims 32-33, further comprising a granulocyte-macrophage colony-stimulating factor (GM-CSF), a granulocyte colony-stimulating factor (G-CSF), a growth hormone, and serotonin, vitamin C, vitamin D, glutamine (Gln), arachidonic acid, AGE-albumin, an interleukin, TNF-alpha, Flt-3 ligand, thrombopoietin, foetal bovine serum (FBS), retinoic acid, lipopolysaccharide (LPS), IFN-gamma, IFN-beta, or combinations thereof.

35. The pharmaceutical composition according to any one of claims 32–34, further comprising a granulocyte-macrophage colony-stimulating factor (GM-CSF) and IFN-gamma.

36. A kit comprising:
a. the granulocyte according to claim 29, the stem cell according to any one of claims 21–23, the composition according to claim 30 or 31, or the pharmaceutical composition according to any one of claims 32–35; and
b. instructions for use of the same in medicine.

37. A granulocyte according to claim 29, stem cell according to any one of claims 21–23, composition according to claim 30 or 31, pharmaceutical composition according to any one of claims 32–35, or kit according to claim 36 for use in treating cancer.

38. Use of the granulocyte according to claim 29, the stem cell according to any one of claims 21–23, the composition according to claim 30 or 31, the pharmaceutical composition according to any one of claims 32–35, or the kit according to claim 36 in the manufacture of a medicament for treating cancer.

39. A method for treating cancer comprising: administering to a subject in need thereof the granulocyte according to claim 29, the stem cell according to any one of claims 21-23, the composition according to claim 30 or 31, the pharmaceutical composition according to any one of claims 32–35, or the kit according to claim 36.

40. The granulocyte for use, stem cell for use, composition for use, pharmaceutical composition for use, or kit for use according to claim 37, the use according to claim 38 or the method according to claim 39, wherein the cancer is one or more of: pancreatic cancer, liver cancer, oesophageal cancer, stomach cancer, cervical cancer, ovarian cancer, lung cancer, bladder cancer, kidney cancer, brain cancer, prostate cancer, myeloma cancer, non-Hodgkin’s lymphoma (NHL), larynx cancer, uterine cancer, or breast cancer; preferably wherein the cancer is pancreatic cancer.

41. A cell bank comprising the granulocyte according to claim 29, the stem cell according to any one of claims 21–23, the composition according to claim 30 or 31, or the pharmaceutical composition according to any one of claims 32–35.
